(19) United States
(12) Patent Application Publication
(10) Pub. No.: US 2012/0164137 A1
(43) Pub. Date: Jun. 28, 2012

(54) ANTI-FOLATE RECEPTOR ALPHA ANTIBODY GLYCOFORMS

(75) Inventors: Philip M. Sass, Audubon, PA (US); Nicholas Nicolaides, Glen Mills, PA (US); Luigi Grasso, Bryn Mawr, PA (US); Eric Routhier, Glen Mills, PA (US); Wei Gu, Malvern, PA (US); Jason Young, Exton, PA (US); Jun Yao, Chester Springs, PA (US)

(73) Assignee: MORPHOTEK, INC., Exton, PA (US)

(21) Appl. No.: 13/277,161

(22) Filed: Oct. 19, 2011

Related U.S. Application Data
(60) Provisional application No. 61/394,812, filed on Oct. 20, 2010.

(51) Int. Cl. A61K 39/395; C12N 5/10; A61P 35/02; A61P 35/00; G01N 33/566; G01N 33/574; C07K 16/28; C12P 21/08

(52) U.S. Cl. 424/133.1; 530/387.3; 435/328; 435/69.6; 435/7.21; 435/7.23; 436/501

(57) ABSTRACT

The invention provides anti-FRA antibodies with novel N-linked neutral glycan profiles in that the relative amounts of one or more neutral glycans are increased or decreased compared to anti-FRA antibodies produced under reference cell culture conditions. The invention further provides anti-FRA antibodies with altered binding to FRA, altered antibody-dependent cellular cytotoxicity (ADCC) and/or altered rate and/or efficiency of internalization in a cell expressing FRA. In related aspects, the invention provides cell cultures comprising an anti-FRA antibody of the invention, a cell isolated from such a culture, kits and compositions comprising an anti-FRA antibody of the invention, methods of producing an anti-FRA antibody of the invention and diagnostic and therapeutic uses of an anti-FRA antibody of the invention.

This application claims the benefit under 35 5 U.S.C. §119(e) of U.S. Provisional Application No. 61/394,812, filed Oct. 20, 2010, which is hereby incorporated by reference herein in its entirety.

### BACKGROUND OF THE INVENTION

Membrane-bound folate receptors hind and transport the vitamin folate into cells. There are three major isoforms of the membrane-bound folate receptor: α, β and γ. “Folate receptor alpha,” “FRA” or “FR-α” refers to the a isoform of the membrane-bound folate receptor. FRA is a single-chain GPI-anchored membrane protein (Kelemen (2006) Int. J. Cancer 119:243-250). The α and β isoforms have about 70% amino acid sequence homology and differ dramatically in their stereospecificity for some folates. Both isoforms are expressed in both fetal and adult tissue, although normal tissue generally expresses low to moderate amounts of FR-β(or FRB). FRA, however, is expressed in a subset of normal epithelial cells and is frequently strikingly elevated in a variety or carcinomas (Ross et al. (1994) Cancer 73(9):2432-2443; Rettig et al. (1988) Proc. Natl. Acad. Sci. USA 85:3110-3114; Campbell et al. (1991) Cancer Res. 51:5329-5338; Coney et al. (1991) Cancer Res. 51:6125-6132; Weitman et al. (1992) Cancer Res. 52:3396-3401; Garin-Chesa et al. (1993) Am. J. Pathol. 142:557-567; Holm et al. (1994) APMIS 102:413-419; Franklin et al. (1994) Int. J. Cancer 8 (Suppl.):89-95; Miotti et al. (1987) Int. J. Cancer 39:297-303; and Vegglan et al. (1989) Tumori 75:510-513). FRA is overexpressed in greater than 90% of ovarian carcinomas (Sudimack and Lee (2000) Adv. Drug Deliv. Rev. 41(2):147-62).

Accordingly, there is a need for anti-human-IRA antibodies, particularly for use to treat FRA-mediated disease, such as FRA-mediated cancer. In particular, there is a need for anti-FRA antibodies with different properties and for methods for making such antibodies and methods for adapting the functional characteristics of the antibodies for various therapeutic and diagnostic uses. For example, the binding affinity, antibody-dependent cellular cytotoxicity, internalization efficiency and/or internalization rate of an anti-FRA antibody can be altered for a desired use.

### SUMMARY OF THE INVENTION

In one aspect, the invention relates to anti-human FRA monoclonal antibodies, particularly the MORAb-003 monoclonal antibody, with different N-linked neutral glycan profiles and/or different properties and to methods for making and using such antibodies by replacing glucose with galactose as a sugar source, reducing the temperature, reducing the dissolved oxygen (DO) level, increasing the CO₂ level, adding CuCl or sodium butyrate to the culture medium or increasing the osmolarity or harvesting the anti-FRA antibody after culture for different lengths of time. The invention also relates to an anti-human FRA antibody, particularly the MORAb-003 antibody, with altered binding affinity, antibody-dependent cellular cytotoxicity, internalization efficiency and/or internalization rate, and to methods for producing such antibodies by replacing glucose with galactose as a sugar source, reducing the temperature, reducing the dissolved oxygen (DO) level, increasing the CO₂ level, adding CuCl or sodium butyrate to the culture medium or increasing the osmolarity or harvesting the anti-FRA antibody after culture for different lengths of time. In another aspect, the invention relates to anti-human FRA antibodies produced by any of the methods described herein and compositions comprising said antibodies. In another aspect, the invention relates to cell cultures engineered to express the heavy and light chains of an anti-human FRA antibody, particularly the MORAb-003 antibody, wherein the cell culture conditions comprise a parameter selected from galactose supplementation, reduced DO, reduced temperature, sodium butyrate or copper chloride supplementation, high osmolarity and high CO₂. The invention also relates to host cells isolated from such cell cultures.

Particular non-limiting embodiments of the invention are set forth in the following numbered paragraphs.

1. A method for producing an anti-human Folate Receptor Alpha (FRA) antibody with a desired N-linked neutral glycan profile in a host cell, wherein said host cell comprises a nucleic acid encoding said anti-human FRA antibody, said method comprising the steps of:

(1) culturing the host cell for a first time period using glucose as a sugar source; and (2) culturing the host cell for a second time period using galactose as a sugar source.

2. The method of embodiment 1, wherein the host cell is cultured using galactose as a sugar source from day 0 to day 14 from the initiation of culture.

3. The method of embodiment 1, wherein the second time period begins at a day selected from day 2 to day 10 from the initiation of culture.

4. The method of embodiment 1, wherein the second time period begins at a day selected from day 5 to day 7 from the initiation of culture.

5. A method for producing an anti-human FRA antibody with reduced binding affinity, comprising the steps of culturing a host cell comprising a nucleic acid encoding the anti-human FRA antibody, for a first time period using glucose as a sugar source; and then culturing said host cell for a second time period using galactose as a sugar source, wherein the anti-human FRA antibody has reduced binding affinity compared to the binding affinity of the anti-human FRA antibody produced by culturing said host cell without galactose.

6. The method of embodiment 5, wherein the binding affinity of the anti human FRA antibody is reduced by at least 10%.

7. The method of embodiment 5, wherein the binding affinity of the anti-human FRA antibody is reduced by at least 15%.

8. A method for producing an anti-human FRA antibody with reduced ADCC, comprising the steps of culturing a host cell comprising a nucleic acid encoding the anti-human FRA antibody, for a first time period using glucose as a sugar source; and then for a second time period using galactose as a sugar source, wherein the anti-human FRA antibody has reduced ADCC compared to the ADCC of the antibody produced by culturing said host cell without galactose.

9. The method of embodiment 8, wherein the ADCC of the anti-human FRA antibody is reduced by at least 5%.

10. The method of embodiment 8, wherein the binding affinity of the anti-human FRA antibody is reduced by at least 65%.

11. The method of any one of embodiments 1, 5 or 8, wherein said host cell is a mammalian host cell.

12. The method of embodiment 11, wherein said mammalian host cell is a recombinant cell derived from a GS CHO (Chinese hamster ovary) cell line.

13. The method of any one of embodiments 1, 5 or 8, wherein said glucose concentration is between 1 g/L to 4 g/L.

14. The method of any one of embodiments 1, 5 or 8, wherein said galactose concentration is 0.01 g/L to 20 g/L.

15. The method of embodiment 14, wherein said galactose concentration is 1 g/L to 10 g/L.

16. The method of embodiment 14, wherein said galactose concentration is 2 g/L to 4 g/L.

17. A method for producing an anti-human FRA antibody comprising the step of culturing a host cell comprising a nucleic acid encoding the antibody, wherein at least a portion of the culturing is done using galactose as a sugar source.

18. The method of embodiment 17, wherein the portion of culturing done using galactose as a sugar source is from day 0 to day 14 from the initiation of culture.

19. The method of embodiment 17, wherein the portion of culturing done using galactose as a sugar source begins at a day selected from day 2 to day 10 from the initiation of culture.

20. The method of embodiment 17, wherein the portion of culturing done using galactose as a sugar source begins at a day selected from day 3 to day 7 from the initiation of culture.

21. The method of embodiment 17, wherein the galactose concentration is 0.01 g/L to 20 g/L.

22. The method of embodiment 17, wherein the galactose concentration is 1 g/L to 10 g/L.

23. The method of embodiment 17, wherein the galactose concentration is 2 g/L to 4 g/L.

24. The method of embodiment 1, wherein the N-linked neutral glycan profile of the anti-human FRA antibody comprises 0-6.3% G0, 21-68% G0F, 24-63% G1F, 0-0.8% G2, 3-11% G2F, 0-0.39% M3N2, 0-0.35% M3N2F, and 0-5% MAN5.

25. The method of embodiment 1, wherein the N-linked neutral glycan profile of the anti-human FRA antibody comprises M3N2:M3N2F:NA2:NA2F:MAN5:NGA2:NA2G1F:NGA2F in a ratio of about 1:1:1.7:60:20:16:365:370.

26. The method of any one of embodiments 1, 5, 8 or 17, wherein the anti-human FRA antibody comprises a heavy chain amino acid sequence comprising SEQ ID NO: 1 and a light chain amino acid sequence comprising SEQ ID NO: 2 or a sequence 99% identical to SEQ ID NO: 2.

27. The method of embodiment 1, wherein the N-linked neutral glycan profile is the profile obtained using an antibody comprising a heavy chain amino acid sequence comprising SEQ ID NO: 1 and a light chain amino acid sequence comprising SEQ ID NO: 2 or a sequence 99% identical to SEQ ID NO: 2 in a CHO cell.

28. The method of embodiment 5, wherein the anti-human FRA antibody has a relative binding affinity of 81.3-88.3% compared to the binding affinity of the antibody produced by culturing said host cell without galactose.

29. The method of embodiment 8, wherein the anti-human FRA antibody induces ADCC in the range 39.0-97.4% compared to the ADCC of the antibody produced by culturing said host cell without galactose.

30. A method for producing an anti-human FRA antibody with a desired N-linked neutral glycan profile in a host cell, wherein said host cell comprises a nucleic acid encoding said anti-human FRA antibody, said method comprising the steps of:

(1) culturing the host cell at a first temperature; and then (2) culturing the host cell at a second temperature lower than the first temperature.

31. The method of embodiment 30, wherein the host cell is cultured at the lower temperature starting at a day selected from day 2 to day 10 from the initiation of culture.

32. The method of embodiment 30, wherein the host cell is cultured at the lower temperature starting at a day selected from day 3 to day 5 from the initiation of culture.

33. A method for producing an anti-human FRA antibody with an increased internalization rate, comprising the steps of culturing a host cell comprising a nucleic acid encoding the anti-human FRA antibody, at a first temperature; and then culturing the host cell at a second temperature lower than the first temperature, wherein the anti-human FRA antibody has an increased internalization rate compared to the internalization rate of the antibody produced by culturing said host cell at the first temperature.

34. The method of embodiment 33, wherein the internalization rate of the anti-human FRA antibody is increased by at least 15%.

35. The method of embodiment 33, wherein the internalization rate of the anti-human FRA antibody is increased by at least 25%.

36. A method for producing an anti-human FRA antibody with an increased internalization efficiency, comprising the steps of culturing a host cell comprising a nucleic acid encoding the anti-human FRA antibody, at a first temperature; and then culturing the host cell at a second temperature lower than the first temperature, wherein the antibody has a reduced internalization efficiency compared to the internalization efficiency of the antibody produced by culturing said host cell at the first temperature.

37. The method of embodiment 36, wherein the internalization efficiency of the anti-human FRA antibody is reduced by at least 15%.

38. The method of embodiment 36, wherein the internalization efficiency of the anti-human FRA antibody is reduced by at least 75%.

39. The method of any one of embodiments 30, 33, 36 or 230, wherein said host cell is a mammalian cell.

40. The method of embodiment 39, wherein said mammalian host cell is a recombinant cell derived from a GS CHO (Chinese hamster ovary) cell line.

41. The method of any one of embodiments 30, 33, 36 or 230, wherein said first temperature at which the host cell is grown is about 36 to 38° C.

42. The method of any one of embodiments 30, 33, 36 or 230, wherein said second temperature is 28 to 35° C.

43. The method of embodiment 42, wherein said second temperature is 30 to 33° C.

44. The method of embodiment 42, wherein said second temperature is 30 to 31° C.

45. A method for producing an anti-human FRA antibody comprising the step of culturing a host cell comprising a nucleic acid encoding the antibody, wherein at least a portion of the culturing is done at low temperature.

46. The method of embodiment 45, wherein the portion of culturing done at low temperature is from day 0 to day 14 from the initiation of culture.

47. The method of embodiment 45, wherein the portion of culturing done at low temperature starts at a day selected from day 2 to day 10 from the initiation of culture.

48. The method of embodiment 45, wherein the portion of culturing done at low temperature starts at a day selected from day 3 to day 5 from the initiation of culture.

49. The method of embodiment 45, wherein the low temperature is 28 to 35° C.

50. The method of embodiment 45, wherein the low temperature is 30 to 33° C.

51. The method of embodiment 45, wherein the low temperature is 30 to 31° C.

52. The method of embodiment 30, wherein the N-linked glycan profile of the anti-human FRA antibody comprises 0-6.3% G0, 21-68% G0F, 24-63% G1F, 0-0.8% G2, 3-11% G2F, 0-0.39% M3N2, 0-0.35% M3N2F, and 0-5% MAN5.

53. The method of embodiment 33, wherein the anti-human FRA antibody internalizes into target cell at a rate of 117-143% compared to the internalization rate of the antibody produced at said first temperature alone.

54. The method of any one of embodiments 30, 33, 36, 45 or 230, wherein the anti-human HU antibody comprises a heavy chain amino acid sequence comprising SEQ ID NO: 1 and a light chain amino acid sequence comprising SEQ ID NO: 2 or a sequence 99% identical to SEQ ID NO: 2.

55. A method for producing an anti-human FRA antibody with a desired N-linked neutral glycan profile in a host cell, wherein said host cell comprises a nucleic acid encoding said anti-human FRA antibody, said method comprising the steps of:

(1) culturing the host cell in cell culture medium at normal osmolarity; and then (2) culturing the host cell in high osmolarity cell culture medium.

56. A method for producing an anti-human HU antibody with reduced binding affinity, comprising the steps of culturing a host cell comprising a nucleic acid encoding the anti-human FRA antibody, in cell culture medium at normal osmolarity; and then culturing the host cell in high osmolarity cell culture medium.

57. The method of embodiment 56, wherein the binding affinity of the anti-human FRA antibody is reduced by at least 25% compared to the binding affinity of the antibody produced by culturing said host cell in culture medium at normal osmolarity.

58. The method of embodiment 56, wherein the binding affinity of the anti-human FRA antibody is reduced by at least 40% compared to the binding affinity of the antibody produced by culturing said host cell in culture medium at normal osmolarity.

59. A method for producing an anti-human FRA antibody with reduced ADCC, comprising the steps of culturing a host cell comprising a nucleic acid encoding the anti-human FRA antibody, in cell culture medium at normal osmolarity; and then culturing the host cell in high osmolarity cell culture medium.

60. The method of embodiment 59, wherein the ADCC of the anti-human FRA antibody is reduced by at least 50% compared to the binding affinity of the antibody produced by culturing said host cell in culture medium at normal osmolarity.

61. The method of embodiment 59, wherein the ADCC of the anti-human FRA antibody is reduced by at least 65% compared to the binding affinity of the antibody produced by culturing said host cell in culture medium at normal osmolarity.

62. A method for producing an anti-human FRA antibody with an increased internalization rate, comprising the steps of culturing a host cell comprising a nucleic acid encoding the anti-human FRA antibody, in cell culture medium at normal osmolarity; and then culturing the host cell in high osmolarity cell culture medium.

63. The method of embodiment 62, wherein the internalization rate of the anti-human FRA antibody is increased by at least 5% compared to the binding affinity of the antibody produced by culturing said host cell in culture medium at normal osmolarity.

64. The method of embodiment 62, wherein the internalization rate of the anti-human FRA antibody is increased by at least 10% compared to the binding affinity of the antibody produced by culturing said host cell in culture medium at normal osmolarity.

65. A method for producing an anti-human FRA antibody with reduced internalization efficiency, comprising the steps of culturing a host cell comprising a nucleic acid encoding the anti-human FRA antibody, in cell culture medium at normal osmolarity; and then culturing the host cell in high osmolarity cell culture medium.

66. The method of embodiment 65, wherein the internalization efficiency of the anti-human FRA antibody is reduced by at least 98.6%.

67. The method of any one of embodiments 55, 56, 59, 62 or 65, wherein said host cell is a mammalian cell.

68. The method of embodiment 67, wherein said host cell is a recombinant cell derived from a GS CHO (Chinese hamster ovary) cell line.

69. The method of any one of embodiments 55, 56, 59, 62 or 65, wherein normal osmolarity of said cell culture medium is in the range 250 to 350 mOsm/L.

70. The method of any one of embodiments 55, 56, 59, 62 or 65, wherein the osmolarity of said high osmolarity medium is 360 to 800 mOsm/L.

71. The method of embodiment 70, wherein the osmolarity of said high osmolarity medium is 400 to 650 mOsm/L.

72. A method for producing an anti-human FRA antibody, comprising the step of culturing a host cell comprising a nucleic acid encoding said antibody, wherein at least a portion of the culturing is done at high osmolarity.

73. The method of embodiment 72, wherein the portion of culturing done at high osmolarity is from day 0 to day 14 from the initiation of culture.

74. The method of embodiment 72, wherein the portion of culturing done at high osmolarity starts at a day selected from day 2 to day 10 from the initiation of culture.

75. The method of embodiment 72, wherein the portion of culturing done at high osmolarity starts at a day selected from day 3 to day 5 from the initiation of culture.

76. The method of embodiment 72, wherein the high osmolarity is 360 to 800 mOsm/L.

77. The method of embodiment 72, wherein the high osmolarity is 400 to 650 mOsm/L.

78. The method of embodiment 55, wherein the N-linked neutral glycan profile of the anti-human FRA antibody comprises 0-7% G0, 49-95% G0F, 0-39% G1F, 0-0.7% G2, 0-6% G2F, 0-0.35% M3N2, 0.04-0.46% M3N2F, and 1.2-5.6% MAN5.

79. The method of embodiment 56, wherein the anti-human FRA antibody has a relative binding affinity of 66.8-78.9% compared to the binding affinity of the antibody produced by culturing said host cell in cell culture medium at normal osmolarity.

80. The method of embodiment 62, wherein the anti-human FRA antibody induces ADCC in the range of 101% compared to the ADCC of the antibody produced by culturing said host cell in cell culture medium at normal osmolarity.

81. The method of embodiment 62, wherein the anti-human FRA antibody internalizes into a target cell at a rate of 107% compared to the internalization rate of the antibody produced by culturing said host cell in cell culture medium at normal osmolarity.

82. The method of embodiment 65, wherein the anti-human FRA antibody internalizes into a target cell with an efficiency of 1.40-1.42% compared to the internalization efficiency of the antibody produced by culturing said host cell in cell culture medium at normal osmolarity.

83. The method of any one of embodiments 55, 56, 59, 62, 65 or 72, wherein the anti-human FRA antibody comprises a heavy chain amino acid sequence comprising SEQ ID NO: 1 and a light chain amino acid sequence comprising SEQ ID NO: 2 or a sequence 99% identical to SEQ ID NO: 2.

84. A method for producing a desired N-linked neutral glycan profile of an anti-human FRA antibody in a host cell, wherein said host cell comprises a nucleic acid encoding said anti-human FRA antibody, said method comprising the steps of:

(1) culturing the host cell in normal cell culture medium; and then (2) adding sodium butyrate to the normal cell culture medium.

85. A method for producing an anti-human FRA antibody with reduced binding affinity, comprising the steps of culturing a host cell comprising a nucleic acid encoding the anti-human FRA antibody, in normal cell culture medium; and then adding sodium butyrate to the normal cell culture medium.

86. The method of embodiment 85, wherein the binding affinity of the anti-human FRA antibody is reduced by at least 40% compared to the binding affinity of the antibody produced by culturing said host cell in normal cell culture medium.

87. The method of embodiment 85, wherein the binding affinity of the anti-human FRA antibody is reduced by at least 50% compared to the binding affinity of the antibody produced by culturing said host cell in normal cell culture medium.

88. A method for producing an anti-human FRA antibody with reduced ADCC, comprising the steps of culturing a host cell comprising a nucleic acid encoding the anti-human FRA antibody, in normal cell culture medium; and then adding sodium butyrate to the normal cell culture medium.

89. The method of embodiment 88, wherein the ADCC of the anti-human FRA antibody is reduced by at least 25% compared to the ADCC of the antibody produced by culturing said host cell in normal cell culture medium.

90. The method of embodiment 88, wherein the ADCC of the anti-human FRA antibody is reduced by at least 50% compared to the ADCC of the antibody produced by culturing said host cell in normal cell culture medium.

91. A method for producing an anti-human FRA antibody with reduced internalization efficiency, comprising the steps of culturing a host cell comprising a nucleic acid encoding the anti-human FRA antibody, culturing the host cell in normal cell culture medium; and then adding sodium butyrate to the normal cell culture medium.

92. The method of embodiment 91, wherein the internalization efficiency of the anti-human FRA antibody is reduced by at least 20% compared to the internalization efficiency of the antibody produced by culturing said host cell in normal cell culture medium.

93. The method of embodiment 91, wherein the internalization efficiency of the anti-human FRA antibody is reduced by at least 50% compared to the internalization efficiency of the antibody produced by culturing said host cell in normal cell culture medium.

94. The method of any one of embodiments 84, 85, 88 or 91, wherein said host cell is a mammalian cell.

95. The method of embodiment 94, wherein said host cell is a recombinant cell derived from a GS CHO (Chinese hamster ovary) cell line.

96. The method of any one of embodiments 84, 85, 88 or 91, wherein said normal cell culture medium does not contain sodium butyrate.

97. The method of any one of embodiments 84, 85, 88 or 91, wherein said sodium butyrate concentration is 0.5 mM.

98. The method of any one of embodiments 84, 85, 88 or 91, wherein said sodium butyrate concentration is 10 mM.

99. A method for producing an anti-human FRA antibody comprising the step of culturing a host cell comprising a nucleic acid encoding the antibody, wherein at least a portion of the culturing is done in culture medium comprising sodium butyrate.

100. The method of embodiment 99, wherein the portion of culturing done in culture medium comprising sodium butyrate is from day 0 to day 14 from the initiation of culture.

101. The method of embodiment 99, wherein the portion of culturing done in culture medium comprising sodium butyrate begins at a day selected from day 2 to day 10 from the initiation of culture.

102. The method of embodiment 99, wherein the portion of culturing done in culture medium comprising sodium butyrate begins at a day selected from day 3 to day 7 from the initiation of culture.

103. The method of embodiment 99, wherein the sodium butyrate concentration is 0.01 mM to 90 mM.

104. The method of embodiment 99, wherein the sodium butyrate concentration is 0.01 mM to 16 mM.

105. The method of embodiment 99, wherein the sodium butyrate concentration is 0.5 mM to 10 mM.

106. The method of embodiment 84, wherein the N-linked neutral glycan profile of the anti-human FRA antibody comprises 0-8% G0, 39-86% G0F, 9-48% G1F, 0-1.0% G2, 0-7% G2F, 0-0.41% M3N2, 0.03-0.45% M3N2F, and 0-4.4% MAN5.

107. The method of embodiment 85, wherein the anti-human FRA antibody has a relative binding affinity of 59-64% compared to the binding affinity of the antibody produced by culturing said host cell in normal cell culture medium.

108. The method of embodiment 85, wherein the anti-human FRA antibody has a relative binding affinity of 44-55% compared to the binding affinity of the antibody produced by culturing said host cell in normal cell culture medium.

109. The method of embodiment 88 wherein the anti-human FRA antibody induces antibody-dependent cellular cytotoxicity in the range 26-91% compared to the ADCC the antibody produced by culturing said host cell in normal cell culture medium.

110. The method of embodiment 91 wherein the anti-human FRA antibody internalizes into target cells with an efficiency of 45-84% compared to the internalization efficiency of the antibody produced by culturing said host cell normal cell culture medium.

111. The method of any one of embodiments 84, 85, 88, 91 or 99, wherein the anti-human FRA antibody comprises a heavy chain amino acid sequence comprising SEQ ID NO: 1 and a light chain amino acid sequence comprising SEQ ID NO: 2 or a sequence 99% identical to SEQ ID NO: 2.

112. A method for producing a desired N-linked neutral glycan profile of an anti-human FRA antibody in a host cell, wherein said host cell comprises a nucleic acid encoding said anti-human FRA antibody, said method comprising the steps of:

(1) culturing the host cell at a first dissolved oxygen (DO) concentration; and then (2) culturing the host cell at a second DO concentration that is lower than the first DO concentration.

113. A method for producing an anti-human FRA antibody with reduced binding affinity, comprising the steps of culturing a host cell comprising a nucleic acid encoding the anti-human FRA antibody, at a first DO concentration; and then culturing the host cell at a second DO concentration that is lower than the first DO concentration.

114. The method of embodiment 113, wherein the binding affinity of the anti-human FRA antibody is reduced by at least 30% compared to the binding affinity of the antibody produced by culturing said host cell at the first DO concentration.

115. The method of embodiment 113, wherein the binding affinity of the anti-human FRA antibody is reduced by at least 50% compared to the binding affinity of the antibody produced by culturing said host cell at the first DO concentration.

116. A method for producing an anti-human FRA antibody with increased ADCC, comprising the steps of culturing a host cell comprising a nucleic acid encoding the anti-human FRA antibody, at a first DO concentration; and then culturing the host cell at a second DO concentration that is lower than the first DO concentration.

117. The method of embodiment 116, wherein the ADCC of the anti-human FRA antibody is increased by at least 25% compared to the ADCC of the antibody produced by culturing said host cell at the first DO concentration.

118. The method of embodiment 116, wherein the ADCC of the anti-human FRA antibody is increased by at least 50% compared to the ADCC of the antibody produced by culturing said host cell at the first DO concentration.

119. A method for producing an anti-human FRA antibody with reduced internalization efficiency, comprising the steps of culturing a host cell comprising a nucleic acid encoding the anti-human FRA antibody, at a first DO concentration; and then culturing the host cell at a second DO concentration that is lower than the first DO concentration.

120. The method of embodiment 119, wherein the internalization efficiency of the anti-human FRA antibody is reduced by at least 30% compared to the internalization efficiency of the antibody produced by culturing said host cell at the first DO concentration.

121. The method of embodiment 119, wherein the internalization efficiency of then anti-human FRA antibody is reduced by at least 60% compared to the internalization efficiency of the antibody produced by culturing said host cell at the first DO concentration.

122. The method of any one of embodiments 112, 113, 116 or 119, wherein said host cell is a mammalian cell.

123. The method of embodiment 122, wherein said host cell is a recombinant cell derived from a GS CHO (Chinese hamster ovary) cell line.

124. The method of any one of embodiments 112, 113, 116 or 119, wherein said first dissolved oxygen concentration at which the host cell can grow is between 30% and 100%.

125. The method of any one of embodiments 112, 113, 116 or 119, wherein said second DO concentration is 0% to 25%.

126. A method for producing an anti-human FRA antibody comprising the step of culturing a host cell comprising a nucleic acid encoding the antibody, wherein at least a portion of the culturing is done at low DO.

127. The method of embodiment 126, wherein the portion of culturing done at low DO is from day 0 to day 14 from the initiation of culture.

128. The method of embodiment 126, wherein the portion of culturing done at low DO starts from a day selected from day 2 to day 10 from the initiation of culture.

129. The method of embodiment 126, wherein the portion of culturing done at low DO starts from a day selected from day 3 to day 7 from the initiation of culture.

130. The method of embodiment 126, wherein the low DO is 0% to 30%.

131. The method of embodiment 130, wherein the low DO is 5% to 25%.

132. The method of embodiment 130, wherein the low DO is 5% to 10%.

133. The method of embodiment 112, wherein the N-linked neutral glycan profile of the anti-human FRA antibody comprises 2-11% G0, 32-79% G0F, 12-52% G1F, 0-1.0% G2, 0-7% G2F, 0-0.28% M3N2, 0.01-0.43% M3N2F, and 0.1-4.4% MAN5.

134. The method of embodiment 113, wherein the anti-human FRA antibody has a relative binding affinity of 50-63% compared to the binding affinity of the antibody produced by culturing said host cell at the first DO concentration.

135. The method of embodiment 116, wherein the anti-human FRA antibody induces antibody-dependent cellular cytotoxicity in the range of greater than 100% to greater than or equal to 200% compared to the ADCC of the antibody produced by culturing said host cell at the first DO concentration.

136. The method of embodiment 119, wherein the anti-human FRA internalizes into a target cell with an efficiency of 39-64% compared to the internalization efficiency of the antibody produced by culturing said host cell at the first DO concentration.

137. The method of any one of embodiments 112, 113, 116, 119 or 126, wherein the anti-human FRA antibody comprises a heavy chain amino acid sequence comprising SEQ ID NO: 1 and a light chain amino acid sequence comprising SEQ ID NO: 2 or a sequence 99% identical to SEQ ID NO: 2.

138. A method for producing a desired N-linked neutral glycan profile of an anti-human FRA antibody in a host cell, wherein said host cell comprises a nucleic acid encoding said anti-human FRA antibody, said method comprising the steps of:

(1) culturing the host cell at a first CO₂ concentration; and then (2) culturing the host cell at a second CO₂ concentration that is higher than the first CO₂ concentration.

139. A method for producing an anti-human FRA antibody with reduced binding affinity, comprising the steps of culturing a host cell comprising a nucleic acid encoding the anti-human FRA antibody, at a first CO₂ concentration; and then culturing the host cell at a second CO₂ concentration that is higher than the first CO₂ concentration.

140. The method of embodiment 139, wherein the binding affinity of the anti-human FRA antibody is reduced by at least 25% compared to the binding affinity of the antibody produced by culturing said host cell at the first CO₂ concentration.

141. The method of embodiment 139, wherein the binding affinity of the anti-human FRA antibody is reduced by at least 50% compared to the binding affinity of the antibody produced by culturing said host cell at the first CO₂ concentration.

142. A method for producing an anti-human FRA antibody with reduced ADCC, comprising the steps of culturing a host cell comprising a nucleic acid encoding the anti-human FRA antibody, at a first CO₂ concentration; and then culturing the host cell at a second CO₂ concentration that is higher than the first CO₂ concentration.

143. The method of embodiment 142, wherein the ADCC of the anti-human FRA antibody is reduced by at least 50% compared to the ADCC of the antibody produced by culturing said host cell at the first CO, concentration.

144. The method of embodiment 142, wherein the ADCC of the anti-human FRA antibody is reduced by at least 65% compared to the ADCC of the antibody produced by culturing said host cell at the first CO₂ concentration.

145. A method for producing an anti-human FRA antibody with an increased internalization rate, comprising the steps of culturing a host cell comprising a nucleic acid encoding the anti-human FRA antibody, at a first CO₂ concentration; and then culturing the host cell at a second CO₂ concentration that is higher than the first CO₂ concentration.

146. The method of embodiment 145, wherein the internalization rate of the anti-human FRA antibody is increased by at least 5% compared to the internalization rate of the antibody produced by culturing said host cell at the first CO₂ concentration.

147. The method of embodiment 145, wherein the internalization rate of the anti-human FRA antibody is increased by at least 25% compared to the internalization rate of the antibody produced by culturing said host cell at the first CO₂ concentration.

148. A method for producing an anti-human FRA antibody with reduced internalization efficiency, comprising the steps of culturing a host cell comprising a nucleic acid encoding the anti-human FRA antibody, at a first CO₂ concentration; and then culturing the host cell at a second CO₂ concentration that is higher than the first CO₂ concentration.

149. The method of embodiment 148, wherein the internalization efficiency of the anti-human FRA antibody is reduced by at least 25% compared to the internalization efficiency of the antibody produced by culturing said host cell at the first CO₂ concentration.

150. The method of embodiment 148, wherein the internalization efficiency of the anti-human FRA antibody is reduced by at least 50% compared to the internalization efficiency of the antibody produced by culturing said host cell at the first CO₂ concentration.

151. The method of any one of embodiments 138, 139, 142, 145 or 148, wherein said host cell is a mammalian cell.

152. The method of embodiment 151, wherein said host cell is a recombinant cell derived from a GS CHO (Chinese hamster ovary) cell line.

153. The method of any one of embodiments 138, 139, 142, 145 or 148, wherein said first CO₇ concentration is about 5% or less.

154. The method of any one of embodiments 138, 139, 142, 145 or 148, wherein said higher CO₂ concentration is 10% to 30%.

155. A method for producing an anti-human FRA antibody comprising the step of culturing a host cell comprising a nucleic acid encoding the antibody, wherein at least a portion of the culturing is done at high CO₂ concentration.

156. The method of embodiment 155, wherein the portion of culturing done at high CO₂ concentration is from day 0 to day 14 from the initiation of culture.

157. The method of embodiment 155, wherein the portion of culturing done at high CO₂ concentration starts from a day selected from day 2 to day 10 from the initiation of culture.

158. The method of embodiment 155, wherein the portion of culturing done at high CO, concentration starts from a day selected from day 3 to day 7 from the initiation of culture.

159. The method of embodiment 155, wherein the high CO₂ concentration is 10% to 30%.

160. The method of embodiment 155, wherein the high CO₂ concentration is 20%.

161. The method of embodiment 138, wherein the N-linked neutral glycan profile of the anti-human FRA antibody comprises 0-7% G0, 50-97% G0F, 0-39% G1F, 0-0.7% G2, 0-6% G2F, 0-0.46% M3N2, 0.06-0.48% M3N2F, and 0-3.8% MAN5.
162. The method of embodiment 139, wherein the anti-human FRA antibody has a relative binding affinity of 45-56% compared to the binding affinity of the antibody produced by culturing said host cell at the first CO₂ concentration.

163. The method of embodiment 142, wherein the anti-human FRA antibody induces ADCC in the range of 23-89% compared to the ADCC of the antibody produced by culturing said host cell at the first CO₂, concentration.

164. The method of embodiment 145, wherein the anti-human FRA antibody internalizes into a target cell at a rate of 105-125% compared to the internalization rate of the antibody produced by culturing said host cell at the first CO₂ concentration.

165. The method of embodiment 148, wherein the anti-human FRA antibody internalizes into a target cell with an efficiency of 40-68% compared to the internalization efficiency of the antibody produced by culturing said host cell at the first CO₂ concentration.

166. The method of any one of embodiments 138, 139, 142, 145, 148 or 155, wherein the anti-human FRA antibody comprises a heavy chain amino acid sequence comprising SEQ ID NO: 1 and a light chain amino acid sequence comprising SEQ ID NO: 2 or a sequence 99% identical to SEQ ID NO: 2.

167. A method for producing a desired N-linked neutral glycan structure of an anti-human FRA antibody in an host cell, wherein said host cell comprises a nucleic acid encoding said anti-human FRA antibody, said method comprising the steps of:

(1) culturing the host cell in normal cell culture medium; and then (2) adding copper chloride to the normal cell culture medium.

168. A method for producing an anti-human FRA antibody with reduced binding affinity, comprising the steps of culturing a host cell comprising a nucleic acid encoding the anti-human FRA antibody, culturing the host cell in normal cell culture medium in which the host cell can grow; and then adding copper chloride to the normal cell culture medium.

169. The method of embodiment 168, wherein the binding affinity of the anti-human FRA antibody is reduced by at least 40% compared to the binding affinity of the antibody produced by culturing said host cell in normal cell culture medium.

170. The method of embodiment 168, wherein the binding affinity of the anti-human FRA antibody is reduced by at least 50% compared to the binding affinity of the antibody produced by culturing said host cell in normal cell culture medium.

171. A method for producing an anti-human FRA antibody with reduced ADCC, comprising the steps of culturing a host cell comprising a nucleic acid encoding the anti-human FRA antibody, in normal cell culture medium; and then adding copper chloride to the normal cell culture medium.

172. The method of embodiment 171, wherein the ADCC of the anti-human FRA antibody is reduced by at least 20% compared to the ADCC of the antibody produced by culturing said host cell in normal cell culture medium.

173. The method of embodiment 1711, wherein the ADCC of the anti-human FRA antibody is reduced by at least 80% compared to the ADCC of the antibody produced by culturing said host cell in normal cell culture medium.

174. A method for producing an anti-human FRA antibody with reduced internalization efficiency, comprising the steps of culturing a host cell comprising a nucleic acid encoding the anti-human FRA antibody, in normal cell culture medium; and then adding copper chloride to the normal cell culture medium.

175. The method of embodiment 174, wherein the internalization efficiency of the anti-human FRA antibody is reduced by at least 30% compared to the internalization efficiency of the antibody produced by culturing said host cell in normal cell culture medium.

176. The method of embodiment 174, wherein the internalization efficiency of the anti-human FRA antibody is reduced by at least 60% compared to the internalization efficiency of the antibody produced by culturing said host cell in normal cell culture medium.

177. The method of any one of embodiments 167, 168, 171 or 174, wherein said host cell is a mammalian cell.

178. The method of embodiment 177, wherein said host cell is a recombinant cell derived from a GS CHO (Chinese hamster ovary) cell line.

179. The method of any one of embodiments 167, 168, 171 or 174, wherein said normal cell culture medium does not contain copper chloride.

180. The method of any one of embodiments 167, 168, 171 or 174, wherein said copper chloride concentration is 0.01 μM to 0.5 mM.

181. A method for producing an anti-human FRA antibody comprising the step of culturing a host cell comprising a nucleic acid encoding the antibody, wherein at least a portion of the culturing is done in culture medium comprising copper chloride.

182. The method of embodiment 181, wherein the portion of culturing done in culture medium comprising copper chloride is from day 0 to day 14 from the initiation of culture.

183. The method of embodiment 181, wherein the portion of culturing done in culture medium comprising copper chloride begins at a day selected from day 2 to day 10 from the initiation of culture.

184. The method of embodiment 181, wherein the portion of culturing done in culture medium comprising copper chloride begins at a day selected from day 3 to day 6 from the initiation of culture.

185. The method of embodiment 181, wherein the copper chloride concentration is 0.01 μM to 0.5 mM.

186. The method of embodiment 181, wherein the copper chloride concentration is 0.01 mM to 0.5 mM.

187. The method of embodiment 181, wherein the copper chloride concentration is 0.5 mM.

188. The method of embodiment 167, wherein the N-linked neutral glycan profile of the anti-human FRA antibody comprises 0-8% G0, 34-81% G0F, 13-53% G1F, 0-0.7% G2, 0-7% G2F, 0.07-0.61% M3N2, 0.16-0.58% M3N2F, and 0-4.3% MAN5.

189. The method of embodiment, wherein the anti-human FRA antibody has a relative binding affinity of 41-56% compared to the binding affinity of the antibody produced by culturing said host cell in normal cell culture medium.

190. The method of embodiment 171, wherein the anti-human FRA antibody induces antibody-dependent cellular cytotoxicity in the range of 21-87% compared to the ADCC of the antibody produced by culturing said host cell in normal cell culture medium.

191. The method of embodiment 174, wherein the anti-human FRA antibody internalizes into target cell with an efficiency of 41-71% compared to the internalization efficiency of the antibody produced by culturing said host cell in normal cell culture medium.

192. The method of any one of embodiments 167, 168, 171, 174 or 181, wherein the anti-human FRA antibody comprises a heavy chain amino acid sequence comprising SEQ ID NO: 1 and a light chain amino acid sequence comprising SEQ ID NO: 2 or a sequence 99% identical to SEQ ID NO: 2.

193. A method of altering one or more properties of an anti-human FRA antibody selected from the group consisting of:

a) the neutral N-glycan profile of the anti-human FRA antibody;

b) the binding affinity;

c) the ADCC;

d) the internalization rate; and

e) the internalization efficiency;

comprising the steps of culturing a host cell comprising a nucleic acid encoding said anti-human FRA antibody, and harvesting a host cell producing the anti-human FRA antibody at a time that is less than 13 days or more than 15 days after initiation of culture.

194. The method of embodiment 193, wherein the binding affinity of the anti-human FRA antibody is reduced compared to the binding affinity of the antibody produced by cells harvested 13-15 days after initiation of culture.

195. The method of embodiment 193, wherein the binding affinity of the anti-human FRA antibody is reduced by at least 25% compared to the binding affinity of the antibody produced by cells harvested 13-15 days after initiation of culture.

196. The method of embodiment 193, wherein the binding affinity of the anti-human FRA antibody is reduced by at least 50% compared to the binding affinity of the antibody produced by cells harvested 13-15 days after initiation of culture.

197. The method of embodiment 193, wherein the ADCC of the anti-human FRA antibody is reduced compared to the antibody harvested at 13-15 days after initiation of culture.

198. The method of embodiment 194, wherein the ADCC of the anti-human FRA antibody is reduced by at least 50% compared to the antibody harvested at 13-15 days after initiation of culture.

199. The method of embodiment 195, wherein the ADCC of the anti-human FRA antibody is reduced by at least 65% compared to the antibody harvested at 13-15 days after initiation of culture.

200. The method of embodiment 193, wherein the internalization rate of the anti-human FRA antibody is increased compared to the antibody harvested at 13-15 days after initiation of culture.

201. The method of embodiment 193, wherein the internalization rate of the anti-human FRA antibody is increased by at least 5% compared to the antibody harvested at 13-15 days after initiation of culture.

202. The method of embodiment 193, wherein the internalization rate of the anti-human FRA antibody is increased by at least 25% compared to the antibody harvested at 13-15 days after initiation of culture.

203. The method of embodiment 193, wherein the internalization efficiency of the anti-human FRA antibody is reduced compared to the antibody harvested at 13-15 days after initiation of culture.

204. The method of embodiment 193, wherein the internalization efficiency of the anti-human FRA antibody is reduced by at least 25% compared to the antibody harvested at 13-15 days after initiation of culture.

205. The method of embodiment 193, wherein the internalization efficiency of the anti-human FRA antibody is reduced by at least 50% compared to the antibody harvested at 13-15 days after initiation of culture.

206. A method for producing an anti-human FRA antibody comprising the step of culturing a host cell comprising a nucleic acid encoding the antibody, wherein the host cell is harvested at a time before 13 days or a time after 15 days from initiation of culture.

207. The method of embodiment 193 or 206, wherein said host cell is a mammalian cell.

208. The method of embodiment 207, wherein said host cell is a recombinant cell derived from a GS CHO (Chinese hamster ovary) cell line.

209. The method of embodiment 193 or 206, wherein said harvesting time is selected from the group consisting of: a time 240 hours-312 hours after initiation of culture and a time 360 hours-480 hours after initiation of culture.

210. The method of embodiment 209, wherein said harvesting time is 240 hours after initiation of culture.

211. The method of embodiment 209, wherein said harvesting time is 408 hours after initiation of culture.

212. The method of embodiment 209, wherein said harvesting time is 480 hours after initiation of culture.

213. The method of embodiment 193 or 206, wherein the anti-human FRA antibody comprises a heavy chain amino acid sequence comprising SEQ ID NO: 1 and a light chain amino acid sequence comprising SEQ ID NO: 2 or a sequence 99% identical to SEQ ID NO: 2.

214. A cell culture comprising a eukaryotic host cell engineered to express the heavy and light chains of an anti-human FRA antibody, wherein the cell culture conditions comprise a parameter selected from the group consisting of: galactose supplementation, reduced dissolved oxygen (DO), reduced temperature, sodium butyrate, copper chloride, high osmolarity and high CO₂.

215. The cell culture of embodiment 214, wherein the DO level is 0% to about 25%.

216. The cell culture of embodiment 214, wherein the CO₂ concentration is about 10% to about 30%.

217. The cell culture of embodiment 214, wherein the temperature is from: 28° C. to about 35° C.

218. The cell culture of embodiment 214, wherein the galactose concentration is 0.01 g/L to 20 g/L.

219. The cell culture of embodiment 214, wherein the sodium butyrate concentration is 0.5 mM to 10 mM.

220. The cell culture of embodiment 214, wherein the copper chloride concentration is 0.01 μM to 0.5 mM.

221. The cell culture of embodiment 214, wherein the osmolarity of the cell culture is 360-800 mOsm/L.

222. The cell culture of embodiment 214, wherein the eukaryotic host cell is a CHO cell.

223. The cell culture of embodiment 222, wherein the CHO cell is a CHO-K1 cell.

224. The cell culture of embodiment 214, wherein the anti-human FRA antibody comprises a human heavy chain constant region of the IgG1 isotype.

225. The cell culture of embodiment 214, wherein the anti-human FRA antibody comprises a heavy chain amino acid sequence comprising SEQ ID NO: 1 and comprises a light chain amino acid sequence comprising SEQ ID NO: 2 or a sequence 99% identical to SEQ ID NO: 2.

226. A host cell isolated from the cell culture of embodiment 214.

227. An anti-human FRA alpha antibody produced by the method of any one of embodiments 1, 5, 8, 17, 30, 33, 36, 45, 55, 56, 59, 62, 65, 72, 85, 88, 91, 99, 113, 116, 119, 126, 139, 142, 145, 148, 155, 168, 171, 174, 181, 206 or 230, or an antigen-binding portion of said antibody.

228. A composition comprising the anti-human FRA antibody of embodiment 227 and a pharmaceutically acceptable carrier.

229. The composition of embodiment 228, further comprising an additional therapeutic agent.

230. A method for producing an anti-human FRA antibody with increased ADCC, comprising the steps of culturing a host cell comprising a nucleic acid encoding the anti-human FRA antibody, at a first temperature; and then culturing the host cell at a second temperature tower than the first temperature.

231. A host cell isolated from the cell culture of embodiment 225.

232. The anti-human FRA alpha antibody of embodiment 227, wherein the anti-human FRA antibody comprises a heavy chain amino acid sequence comprising SEQ ID NO: 1 and comprises a light chain amino acid sequence comprising SEQ ID NO: 2 or a sequence 99% identical to SEQ ID NO: 2.

233. A composition comprising the anti-human FRA antibody of embodiment 232 and a pharmaceutically acceptable carrier.

### BRIEF DESCRIPTION OF THE DRAWINGS

FIG. 1A-1H shows diagrams of neutral N-linked antibody glycan structures recovered from anti-FRA antibodies. The figure depicts partially processed glycan structures M3N2 (FIG. 1A), M3N2F (FIG. 1B), MAN5 (FIG. 1C) and the fully processed glycan structures NGA2 (G0) (FIG. 1D), NGA2F (G0F) (FIG. 1E), NA2G1F (G1F) (FIG. 1F), NA2 (G2) (FIG. 1G) and NA2F (G2F) (FIG. 1H).

FIG. 2 is a graph showing the distribution of neutral N-linked antibody glycan structures recovered from anti-FRA antibodies produced by host cells cultured under various conditions. “Pos. ctl.” represents anti-FRA antibodies produced by host cells cultured at conditions listed as “Positive Control” in Table 3. “MORAb-003 reference standard” represents an anti-FRA antibody having a heavy chain amino acid sequence comprising the amino acid sequence of SEQ ID NO: 5 and a light chain amino acid sequence comprising the amino acid sequence in SEQ ID NO: 7 produced under “reference culture conditions” as defined herein and supplied by an outside manufacturer. Host cell culture conditions corresponding to “galactose suppl.,” “low temp.,” “high osmo.,” “0.5 mM Na butyrate,” “low DO,” “high CO₂,” “10 ml Na butyrate,” “CuCl suppl.,” “Day 10 harvest,” “Day 14 harvest,” “Day 17 harvest” and “Day 20 harvest” are described in Example 1.

FIG. 3 is a table showing the distribution of neutral N-linked antibody glycan structures recovered from anti-FRA antibodies produced by host cells cultured under various conditions. “MORAb-003 reference standard” is the antibody as described in connection with FIG. 2.

FIG. 4 is a table showing the FRA binding affinity of anti-FRA antibodies produced by host cells cultured under various conditions.

FIG. 5 is a table showing the ADCC of anti-FRA antibodies produced by host cells cultured under various conditions.

FIG. 6 is a leverage plot showing the correlations between ADCC (y-axis) and the relative concentration (%) of the glycan NGA2 (G0) in an anti-FRA antibody (x-axis).

FIG. 7 is a leverage plot showing the correlations between ADCC (y-axis) and the relative concentration (%) of non-fucosylated glycans in an anti-FRA antibody (x-axis).

FIG. 8 is a leverage plot showing the correlations between ADCC (y-axis) and the relative concentration (%) of the glycan M3N2F in an anti-EPA antibody (x-axis).

FIG. 9 is a graph showing the results of an experiment measuring the internalization of anti-FRA antibodies. The internalization of the anti-FRA antibodies was measured as a function of the inhibition of FRA-expressing cell proliferation by using an anti-human secondary immunotoxin. The OD540 is shown on the y-axis. The concentration of the anti-FRA antibodies is shown on the x-axis. “reference standard” refers to the MORAb-003 antibody described in connection with FIG. 2.

FIG. 10 is a table showing the calculation of the concentrations of anti-FRA antibody resulting in 50% inhibition of FRA-expressing cell proliferation (IC50). “ref std” refers to the MORAb-003 antibody described in connection with FIG. 2.

FIG. 11 is a histogram showing the results of a FACS binding experiment measuring the internalization activity of an anti-FRA antibody. “ref std” refers to the MORAb-003 antibody described in connection with FIG. 2. The shaded area (P1 population) corresponds to cells incubated with FITC-conjugated anti-human IgG antibody. The P2 population (0% control) corresponds to cells incubated with irrelevant human IgG as control and FITC-conjugated anti-human IgG antibody. The P3 population corresponds to cells incubated with the anti-FRA antibody and FITC-conjugated anti-human IgG antibody and washed with acidic glycine buffer. The P4 population (100% control) corresponds to cells incubated with the anti-FRA antibody and FITC-conjugated anti-human IgG antibody with PBS buffer wash.

FIG. 12 is a graph showing the results of a FACS binding experiment measuring the internalization activity of an anti-FRA antibody produced under “reference culture conditions” as defined herein and supplied by an outside manufacturer. The percentage of mean fluorescence intensity (MFI) measured by flow cytometry of a population of FRA-expressing cells (y-axis) is depicted as a function of time (x-axis) relative to total binding at each time point.

FIG. 13 is a graph showing the results of a FACS binding experiment measuring the internalization activity of anti-FRA antibodies described in Table 4, as well as control anti-FRA antibodies. The percentage of MFI measured by flow cytometry of a population of FRA-expressing cells (y-axis) is depicted as a function of time (x-axis) relative to total binding at each time point. “MORAb-003 ref std” refers to the MORAb-003 antibody described in connection with FIG. 2.

FIG. 14 depicts a fit model of the anti-FRA antibody internalization percentage as a function of time, wherein Log(agonist) vs. Response—Variable Slope. “ref std” refers to the MORAb-003 antibody described in connection with FIG. 2.

FIG. 15 is a table summarizing the results of the FACS internalization studies described in Example 5.

FIG. 16 is a table summarizing the data related to the binding affinity, ADCC, internalization rate and internalization efficiency of the anti-FRA antibodies. “MORAb-003 Reference Standard” refers to the MORAb-003 antibody described in connection with FIG. 2.

### DETAILED DESCRIPTION OF THE INVENTION

The invention is based in part on the surprising discovery that by varying certain cell culture conditions for the recombinant production of an anti-FRA antibody, in particular the MORAb-003 anti-FRA monoclonal antibody, one can change the N-linked neutral glycan profile of the antibody and in some cases, change one or more properties of the anti-FRA antibody. In particular, the inventors have discovered that one can change the N-linked neutral glycan profile of an anti-FRA antibody by replacing glucose with galactose as a sugar source, reducing the temperature, reducing the dissolved oxygen (DO) level, increasing the CO₂ level, adding CuCl or sodium butyrate to the culture medium, increasing the osmolarity or harvesting the anti-FRA antibody after culture for different lengths of time and that each of the aforementioned culture conditions alters the N-linked neutral glycan profile. That is, one or more neutral glycans making up the profile are present in increased or decreased amounts relative to the amount of said glycans in the profile of an anti-FRA antibody produced under reference conditions as defined herein. An anti-FRA monoclonal antibody, such as the MORAb-003 antibody, with altered N-linked neutral glycans is useful as an alternative therapeutic molecule because such molecules may have one or more desirable properties including but not limited to altered pK and or pD, altered improved solubility, reduced immunogenicity or reduced side effects. The inventors further discovered that producing the anti-FRA antibody under any of these conditions alters the binding affinity, ADCC, internalization rate and/or internalization efficiency of the anti-FRA antibody.

Accordingly, in one aspect, the invention provides anti-FRA antibodies with novel N-linked neutral glycan profiles in that the relative amounts of one or more neutral glycans is increased or decreased compared to anti-FRA antibodies produced under reference cell culture conditions as defined herein. The invention further provides anti-FRA antibodies with altered (typically reduced) binding to FRA, altered antibody-dependent cellular cytotoxicity (ADCC) and/or altered internalization rate and/or internalization efficiency in a cell expressing FRA. In various embodiments, the anti-FRA antibody comprises the heavy chain amino acid sequence of SEQ ID NO: 1 or SEQ ID NO: 5 or a sequence that is at least 95% identical, and the light chain amino acid sequence of SEQ ID NO: 2 or SEQ ID NO: 6 or SEQ ID NO: 7 or a sequence that is at least 95% identical. In particular embodiments, the antibody comprises the heavy chain amino acid sequence of SEQ ID NO: 1 and a light chain amino acid sequence that is 99% identical to the light chain amino acid sequence of SEQ ID NO: 2, the heavy chain amino acid sequence of SEQ ID NO: 1 and the light chain amino acid sequence of SEQ ID NO: 6, or the heavy chain amino acid sequence of SEQ ID NO: 5, the light chain amino acid sequence of SEQ ID NO: 7, or sequences that are at least 95% identical to the above-mentioned sequences (Ebel et al., (2007) Cancer Immunity, 7:6). In some embodiments, the anti-FRA antibody has a heavy chain amino acid sequence that is at least 96%, at least 97%, at least 98%, or at least 99% identical to SEQ ID NO: 1 or SEQ ID NO: 5 and a light chain amino acid sequence that is at least 96%, at least 97%, at least 98%, or at least 99% identical to SEQ ID NO: 2, SEQ ID NO: 6 or SEQ ID NO: 7. In some embodiments, the anti-FRA antibody comprises a heavy chain encoded by the nucleotide sequence of SEQ ID NO: 8 (with or without the nucleotides encoding the leader sequence) and a light chain encoded by the nucleotide sequence of SEQ ID NO: 9 (with or without the nucleotides encoding the leader sequence). In some embodiments, the heavy chain amino acid sequence lacks the C-terminal lysine. In various embodiments, the anti-FRA antibody of the invention has the amino acid sequences of the antibody produced by a cell line deposited under terms in accordance with the Budapest Treaty with the American Type Culture Collection (ATCC), 10801 University Blvd., Manassas, Va. 20110-2209 on Apr. 24, 2006 under the accession no. PTA-7552 or such sequences lacking the heavy chain C-terminal lysine.

In related aspects, the invention provides cell cultures comprising an anti-FRA antibody of the invention, a cell isolated from such a culture and kits and compositions comprising an anti-FRA antibody of the invention.

In another aspect, the invention provides methods for producing an anti-FRA antibody by altering a cell culture condition, the alteration selected from: using galactose as a sugar source, reducing the temperature, reducing the dissolved oxygen (DO) level, increasing the CO₂ level, adding CuCl or sodium butyrate to the culture medium, increasing the osmolarity or harvesting the anti-FRA antibody after culture for different lengths of time and an anti-FRA antibody produced by the method. The invention further provides methods for altering the N-linked neutral profile and/or one or more properties of an anti-FRA antibody or for producing an anti-FRA antibody with a desired N-linked neutral glycoform profile or property by culturing host cells expressing an anti-FRA antibody under an altered culture condition described herein.

In still a further aspect, the invention provides methods of using an anti-FRA antibody of the invention. In some embodiments, the antibodies are used to detect the presence or to quantitate FRA or cells expressing FRA in vitro or in vivo. In other embodiments, an anti-FRA antibody of the invention is used for therapy either alone or in combination with one or more additional therapeutic agents.

The antibodies of the invention specifically bind to human folate receptor alpha (FRA). As used herein, an antibody that specifically binds FRA (also referred to herein as an anti-FRA antibody) does not bind significantly to proteins that are not FRA. An antibody is said to specifically bind an antigen when the dissociation constant (K_{D}) is ≦1 mM, ≦100 nM or ≦10 nM. In certain embodiments, the K_{D} is 1 pM to 500 pM. In other embodiments, the K_{D} is between 500 pM to 1 μM, 1 μM to 100 nM or 100 mM to 10 nM. In a preferred embodiment, the FRA is human FRA such as the human FRA comprising the amino acid sequence shown in SEQ ID NO: 3 or the amino acid sequence encoded by the nucleotide sequence in SEQ ID NO: 4.

In some embodiments of the invention, an anti-FRA antibody is a four chain antibody (also referred to as an immunoglobulin) comprising two heavy chains and two light chains. The heavy chain of an anti-FRA antibody of the invention is composed of a heavy chain variable domain (V_{H}) and a heavy chain constant region (C_{H}). The light chain is composed of a light chain variable domain (V_{L}) and a light chain constant domain (C_{L}). For the purposes of this application the mature heavy chain and light chain variable domains each comprise three complementarity determining regions (CDR1, CDR2 and CDR3) within four framework regions (FR1, FR2, FR3 and FR4) arranged from N-terminus to C-terminus: FR1, CDR1, FR2, CDR2, FR3, CDR3 and FR4. The assignment of amino acids to each domain herein is in accordance with the definitions of Kabat, Sequences of Proteins of immunological Interest (National institutes of Health, Bethesda, Md. (1987 and 1991)), Chothia & Lesk (1987) J. Mol. Biol. 196:901-917 or Chothia et al., Nature (1989) 342:878-883.

An antibody of the invention is a human immunoglobulin G subtype 1 (IgG1) with a human kappa light chain.

The term “antibody” can refer to an individual antibody molecule or a plurality of antibody molecules, as appropriate for the context. Those of skill in the art will appreciate, for example, that a neutral glycan profile relates to a plurality of antibody molecules.

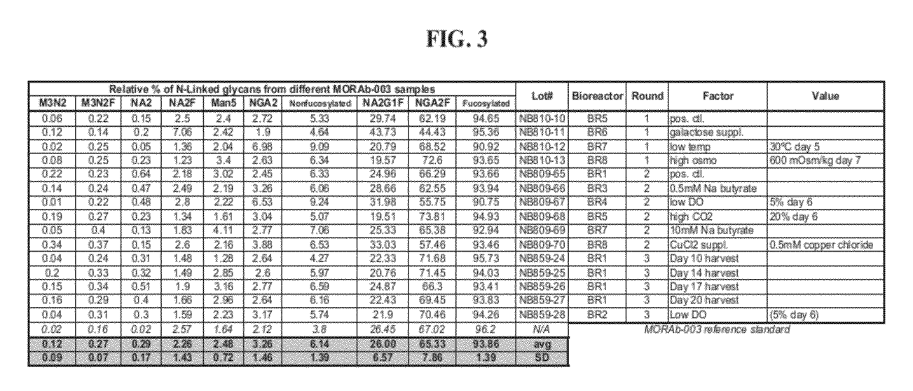

The invention provides, in various embodiments, an anti-FRA antibody having any of the N-linked neutral glycan profiles shown in FIG. 3 or described in the following “Culture Conditions” section. In some embodiments, an anti-FRA antibody of the invention has an N-linked neutral glycan profile that comprises an increased amount of M3N2, NA2, NA2F, MAN5 and NA2G1F and decreased NGA2F compared to the profile of the antibody produced under reference culture conditions. In some embodiments, the N-linked neutral glycan profile of the anti-FRA antibody has an increased amount of NGA2, for example at least a two-fold or at least a three-fold increase in NGA2 compared to the profile of the antibody produced under reference culture conditions. In some embodiments, the anti-ETA antibody comprises about 9% non-fucosylated glycoforms. The invention also provides an anti-FRA antibody having an N-linked neutral glycan profile comprising an increased amount of NA2, NGA2F and MAN5 and a decreased amount of NA2G1F compared to the profile of the antibody produced under reference culture conditions. In some embodiments, the anti-FRA antibody has an N-linked neutral glycan profile that comprises an increased amount of M3N2 and NA2 compared to the profile of the antibody produced under reference culture conditions. In some embodiments, the anti-FRA antibody has an N-linked neutral glycan profile that comprises an increased amount of NA2 and NGA2 compared to the profile of the antibody produced under reference culture conditions. In some embodiments, the anti-FRA antibody has an N-linked neutral glycan profile that comprises an increased amount of M3N2 and NA2 and decreased NA2F compared to the profile of the antibody produced under reference culture conditions. In some embodiments, the anti-FRA antibody has an N-linked neutral glycan profile that comprises an increased amount of M3N2F, NA2 and MAN5 compared to the profile of the antibody produced under reference culture conditions. In some embodiments, the anti-FRA antibody has an N-linked neutral glycan profile that comprises an increased amount of M3N2, M3N2F and NA2 compared to the profile of the antibody produced under reference culture conditions.

In such profiles, the M3N2, M3N2F and MAN5 may be increased by at least about 2-fold or more. The NA2 may be increased by at least about 10-fold or more. The NA2F may be increased by at least about 2-fold or more. The NA2F may be decreased by at least about 40% or more or may be increased by at least about 2-fold or more. The NA2G1F may be decreased by at least about 25% or 30% or more, and the NGA2F may be increased by at least about 10% or 15% or more.

In particular embodiments, the anti-FRA monoclonal antibody is the MORAb-003 monoclonal antibody. “MORAb-003” refers to an anti-FRA antibody that comprises the heavy chain amino acid sequence of SEQ ID NO: 5 and the light chain amino acid sequence of SEQ ID NO: 7 and is produced under reference culture conditions and supplied by an outside manufacturer. Kinetic and steady-state binding constants between the MORAb-003 antibody reference standard and purified FR-α have been determined by surface plasmon resonance spectroscopy. On-rate (kₐ) was determined to be (2.25.+−.0.02) M⁻¹s⁻¹, and off-rate (kd) was determined to be (5.02.+−.0.08) s⁻¹. A steady state dissociation constant (K_{D}) has been determined to be 2.23 nM (United States Patent Publication 20050232919).

In various embodiments, the invention provides an anti-FRA antibody having decreased binding affinity, increased or decreased ADCC, reduced internalization efficiency or increased internalization rate compared to an anti-FRA antibody produced under reference culture conditions.

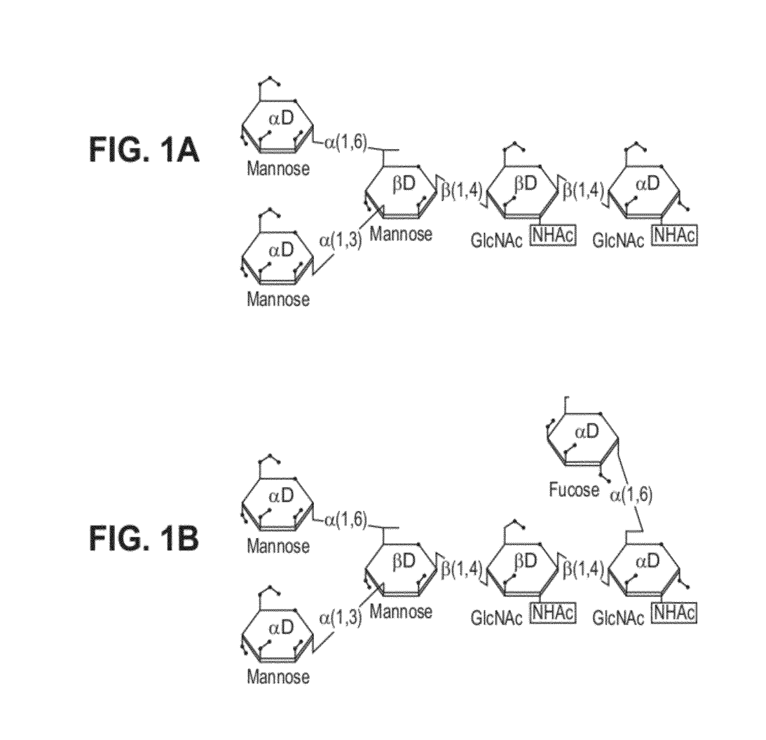

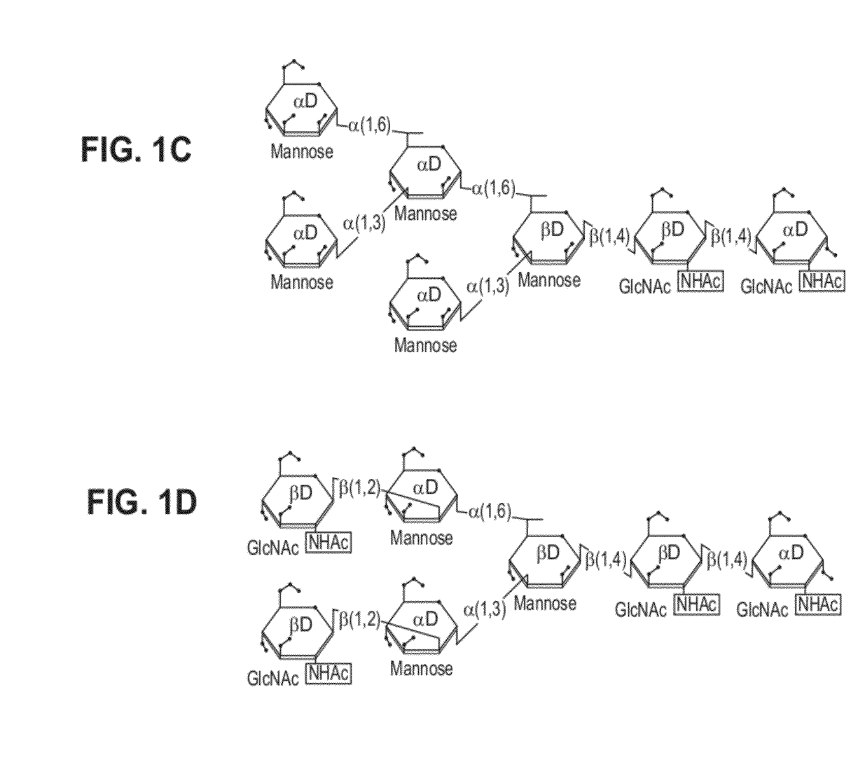

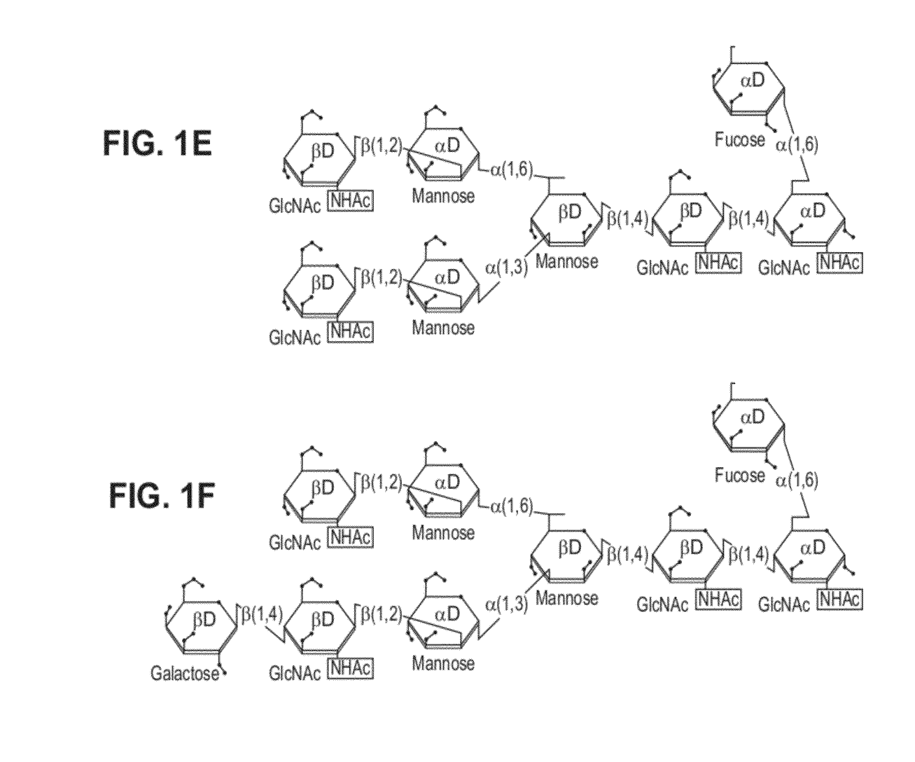

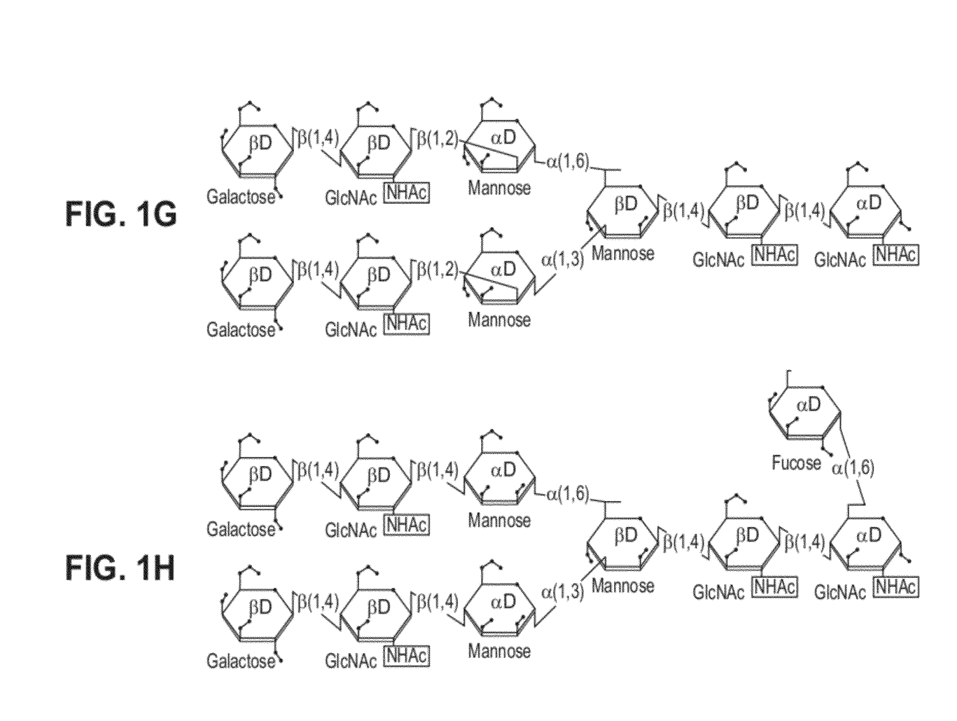

As used herein, N-linked neutral glycans are attached at the conserved glycosylation site or a corresponding position (Asn299 in SEQ ID NO: 5). As used herein, an “N-linked neutral glycan profile” of an anti-FRA antibody comprises neutral glycan structures shown in FIG. 1. Such structures include the partially processed glycans M3N2, M3N2F and MAN5 and the fully processed glycans NGA2 (G0), NGA2F (G0F), NA2G1F (G1F), NA2 (G2) and NA2F (G2F). For the purposes of this application, “G0” refers to agalactosylated glycans, “G1” refers to monogalactosylated glycans and “G2” refers to digalactosylated glycans.

The glycan profile of the antibody of the invention may be determined as described in Example 2. Briefly, N-linked glycans attached to the antibody heavy chain are enzymatically removed using peptidyl-N-glycosidase F (PNGase F) and purified by gel filtration chromatography. The resulting glycan mixture is fluorescently labeled using, for example, 2-aminobenzamide (2-AB), and resolved by normal phase HPLC. Fluorescently labeled glycans are quantified by fluorescence. Identification of glycans from peaks arising during separation is accomplished by in-line mass spectrometric detection. Complex N-linked glycans may be labeled following enzymatic removal using any suitable fluorophores (e.g., 2-aminobenzoic acid (2-AA), 2-aminobenzamide (2-AB), 2-aminopyridine (2-AP), 8-Aminonaphthalene-1,3,6-trisulfonic acid (ANTS), 2-Aminoacridone (AMAC), or 9-Aminopyrene-1,3,6-trisulfonic acid (APTS)), or using radioisotopes, or using small chemical tags that can themselves be detected using other means (e.g., biotin). Complex glycans, whether labeled or unlabeled, may then be separated by a variety of methods, including HPLC (reverse phase, normal phase, anion exchange), and gel-based or capillary electrophoretic methods, and may be enumerated by fluorescence, pulsed amperometric, refractive index, evaporative light scatter or mass spectrometric techniques.

According to the methods of the invention, an anti-FRA antibody of the invention is produced by culturing cells expressing the antibody. In some embodiments, a nucleic acid encoding a heavy chain, a light chain or both is inserted into an expression vector and may be operably linked to expression control sequences such as transcriptional and translational control sequences.

“Operably linked” sequences include both expression control sequences that are contiguous with the gene of interest and expression control sequences that act in trans or at a distance to control the gene of interest. The term “expression control sequence” as used herein means polynucleotide sequences that affect the expression and processing of coding sequences to which they are ligated. Expression control sequences may include appropriate transcription initiation, termination, promoter and enhancer sequences; RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in eukaryotes, generally, such control sequences include promoters and transcription termination sequences. The term “control sequences” is intended to include, at a minimum, all components whose presence is essential for expression and processing, and can also include additional components whose presence is advantageous, for example, leader sequences and fusion partner sequences.

Expression vectors include plasmids, retroviruses, adenoviruses, adeno-associated viruses (AAV), EMI derived episomes, and the like. In some instances, a nucleic acid encoding an antibody, an antibody chain or an antigen-binding fragment of the invention is ligated into a vector such that transcriptional and translational control sequences within the vector serve their intended function of regulating the transcription and translation of the antibody gene. As is well-known to the skilled worker, the expression vector and expression control sequences are chosen to be compatible with the desired level of expression, the expression host cell used and the like. Nucleic acids encoding the antibody light chain or fragment and the antibody heavy chain or fragment can be inserted into separate vectors or into the same expression vector. The nucleic acids are inserted into the expression vector by standard methods (e.g., ligation of complementary restriction sites on the antibody encoding nucleic acid(s) and vector, or blunt end ligation if no restriction sites are present).

Mammalian lines useful as hosts for expression are well known in the art and include many immortalized cell lines available from the American Type Culture Collection (ATCC). These include, inter alia, Chinese hamster ovary (CHO) cells (such as CHO-K1 cells), NS0 cells, SP2 cells, HEK-293T cells, NIH-3T3 cells, HeLa cells, baby hamster kidney (BHK) cells, African green monkey kidney cells (COS), human hepatocellular carcinoma cells (e.g., Hep G2), A549 cells, and a number of other cell lines. Cell lines of particular preference are selected through determining which cell lines have high expression levels. In one embodiment, the cells are not temperature sensitive mutant cell lines. In another embodiment, the cells are temperature sensitive mutant cell lines. In some embodiments, the host cells are CHO cells, CHO-K1 cells or GS CHO-K1 cells.

In some embodiments, a recombinant expression vector encoding an antibody is introduced into mammalian host cells, and the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods.

Further, expression from production cell lines can be enhanced using a number of known techniques. For example, the glutamine synthetase gene expression system (the GS system) is a common approach for enhancing expression under certain conditions. The GS system is discussed in whole or part in connection with European Patent Nos. 216 846, 256 055, 323 997 and 338 841, incorporated herein by reference in their entirety.

In one aspect, the invention provides methods for producing an anti-FRA antibody using a variety of cell culture conditions. As used herein, “reference culture conditions” refers to cells cultured in 2 L stirred-tank production bioreactors in CD-CHO (Invitrogen) medium at 180 rpm. The pH of the reference cell culture is in the range of 6.9-7.1. For example, the pH may be 7.0. The glucose concentration of the reference cell culture is 1 g/l, to 4 g/L, or 1 g/L to 3 g/L. The temperature of the reference cell culture is from about 36 to 38° C. For example, the temperature may be 36.5° C. The CO₂ concentration is about 5%. The reference osmolarity of the cell culture medium is in the range from 250 to 350 mOsm/L. The reference cell culture medium does not contain sodium butyrate or copper chloride. The dissolved oxygen tension (DO) of the reference cell culture medium is in the range of 30-100%. For example, the DO of the reference cell culture medium is between 30% and 40%. The harvesting time for antibodies cultured under reference cell culture conditions may be 13 to 15 days, for example, 14 days (i.e. 336 hours) after initiation of the cell culture, which is expected to be the time at which culture viability is 50%. The “MORAb-003 reference standard,” “MORAb-003 ref std” and “reference standard” samples described in the Figures comprise the heavy chain amino acid sequence in SEQ ID NO: 5 and the light chain amino acid sequence in SEQ ID NO: 7, and were produced by cells cultured under the previously described reference culture conditions and supplied by an outside manufacturer.

In some embodiments, the culture conditions are varied during growth phase and/or lag phase. A batch culture is a population of cells grown in a closed system. The typical growth curve for a population of cells in a batch culture comprises tag phase, exponential phase, stationary phase and death phase. Lag phase refers to the first stage of the growth cycle when a population is taken from an old growth environment to anew environment, where cells are adapting to the new source of nutrients; synthesizing RNA, protein and other molecules but not yet dividing. The length of this period may be brief or extended, depending on the history of the culture and growth conditions.

For example, the invention provides methods for producing an anti-FRA antibody by culturing cells capable of expressing the antibody for at least a portion of the culture time using galactose as a sugar source. In some embodiments, galactose is used as the sugar source from day 0 onward, where day 0 is the day of inoculation. In other embodiments, the cells are cultured for a first time period using glucose as a sugar source, and for a second time period using galactose as a sugar source. For example, according to the methods of the invention, an anti-FRA antibody of the invention is produced by cell culture using glucose as a sugar source in the lag phase and using galactose as a sugar source in the growth phase. In some embodiments, cells are cultured using galactose as a sugar source from day 0 to day 14, or starting any day from 2 to day 10, or preferably starting from day 3, day 4, day 5, day 6 or day 7. For any of the above methods, the galactose concentration may be 0.01 g/L to 20 g/L, preferably 1 g/L to 10 g/L, or more preferably 2 g/L to 4 g/L. In some embodiments, the galactose concentration may be 2 g/L.

According to the invention, an anti-FRA antibody produced by cells cultured using galactose as a sugar source has an N-linked neutral glycan profile comprising increased M3N2, NA2, NA2F, MAN5 and NA2G1F and decreased NGA2F, compared to an anti-FRA antibody reference standard. In some embodiments, an anti-FRA antibody produced by cells cultured using galactose as a sugar source has an N-linked neutral glycan profile comprising decreased M3N2F and/or increased NA2F and/or increased NA2G1F and/or decreased NGA2F, compared to the anti-FRA antibody produced under reference cell culture conditions (Lot No. NB810-10). In some embodiments, the amount of the M3N2F glycoform in the anti-FRA antibody produced by cells cultured using galactose as a sugar source may be at least 30% reduced compared to the anti-FRA antibody produced under reference cell culture conditions (Lot No. NB810-10). In some embodiments, the amount of the NA2F glycoform in the anti-FRA antibody produced by cells cultured using galactose as a sugar source may be at least two-fold increased compared to the anti-FRA antibody produced under reference cell culture conditions (Lot No. NB810-10). In some embodiments, the amount of the NA2G1F glycoform in the anti-FRA antibody produced by cells cultured using galactose as a sugar source may be at least 40% increased compared to the anti-FRA antibody produced under reference cell culture conditions (Lot No. NB810-10). In some embodiments, the amount of the NGA2F glycoform in the anti-FRA antibody produced by cells cultured using galactose as a sugar source may be at least 25% decreased compared to the anti-FRA antibody produced under reference cell culture conditions (Lot No. NB810-10). In some embodiments, the anti-FRA antibody produced by cells cultured using galactose as a sugar source has an N-linked neutral glycan profile comprising a ratio of M3N2:M3N2F:NA2:NA2F:MAN5:NGA2:NA2G1F:NGA2F of about 1:1:1.7:60:20:16:365:370. In some embodiments, the anti-FRA antibody produced by cells cultured using galactose as a sugar source has an N-linked neutral glycan profile of about 0.12% M3N2, 0.14% M3N2F, 0.2% NA2, 7.06% NA2F, 2.42% MAN5, 1.9% NGA2, 43.73% NA2G1F and 44.43% NGA2F. In some embodiments, at least 6% or 7% of the neutral glycans in the anti-FRA antibody produced by cells cultured using galactose as a sugar source may be of the NA2F glycoform. In some embodiments, the anti-FRA antibody produced by cells cultured using galactose as a sugar source has an N-linked neutral glycan profile of about 4.64% non-fucosylated glycoforms and 95.36% fucosylated glycoforms.

The invention further provides methods for producing an anti-FRA antibody by culturing cells capable of expressing the antibody for at least a portion of the culture time at a low temperature. In some embodiments, the cells are cultured at a low temperature from day 0 onward, where day 0 is the day of inoculation. In other embodiments, the cells are cultured for a first time period at a first temperature, and for a second time period at a lower temperature. For example, according to the methods of the invention, an anti-FRA antibody of the invention is produced by cell culture using a first temperature in the lag phase and using a lower temperature in the growth phase. The first temperature may be about 36 to 38° C. The lower temperature may be about 28 to 35° C., or about 30 to 33° C., or about 30 to 31° C. and may be about 28° C., 29° C., 30° C., 31° C., 32° C., 33° C., 34° C. or 35° C. In some embodiments, the cells are cultured fro a first time period at a temperature of 365° C. and for a second time period at 30° C. The cells may be cultured at the lower temperature from day 0 to day 14, or starting any day from 2 to day 10, or preferably starting from day 3, day 4 or day 5.

According to the invention, an anti-FRA antibody produced by cells cultured at a lower temperature has an N-linked neutral glycan profile comprising increased NGA2 compared to the anti-FRA antibody reference standards. According to the invention, an anti-FRA antibody produced by cells cultured at a lower temperature has an N-linked neutral glycan profile comprising increased NGA2 and/or decreased NA2G1F, compared to the anti-FRA antibody produced under reference cell culture conditions (Lot No. NB810-10). In some embodiments, an anti-FRA antibody produced by cells cultured at a lower temperature has an N-linked neutral glycan profile comprising increased non-fucosylated glycoforms, compared to the anti-FRA antibody produced under reference cell culture conditions (Lot No. NB810-10). In some embodiments, the amount of the NGA2 glycoform in the anti-FRA antibody produced by cells cultured at low temperature may be at least two-fold increased compared to the anti-FRA antibody produced under reference cell culture conditions (Lot No. NB810-10). In some embodiments, the amount of the NA2G1F glycoform in the anti-FRA antibody produced by cells cultured at low temperature may be at least 30% decreased compared to the anti-FRA antibody produced under reference cell culture conditions (Lot No, NB810-10). In some embodiments, the anti-FRA antibody cultured at a lower temperature has an N-linked neutral glycan profile comprising a ratio of M3N2:M3N2F:NA2:NA2F:MAN5:NGA2 NA2G1F:NGA2F of about 1:13:2.5:70:100:350:1050:3500. In some embodiments, the anti-FRA antibody produced by cells cultured at a lower temperature has an N-linked neutral glycan profile of about 0.02% M3N2, 0.25% M3N2F, 0.05% NA2, 1.36% NA2F, 2.04% MAN5, 6.98% NGA2, 68.52% NA2G1F and 90.92% NGA2F. In some embodiments, at least 6% or 7% of the neutral glycans in the anti-FRA antibody produced by cells cultured at low temperature may be of the NGA2 glycoform. In some embodiments, the anti-FRA antibody produced by cells cultured at a lower temperature may comprise about 9% non-fucosylated glycoforms. In some embodiments, the anti-FRA antibody produced by cells cultured at tow temperature has an N-linked neutral glycan profile of about 9.09% non-fucosylated glycoforms and 90.92% fucosylated glycoforms.

The invention also provides methods for producing an anti-FRA antibody by culturing cells capable of expressing the antibody for at least a portion of the culture time at a high osmolarity. In some embodiments, the cells are cultured at a high osmolarity from day 0 onward, where day 0 is the day of inoculation. In other embodiments, the cells are cultured for a first time period at a reference osmolarity, and for a second time period at a higher osmolarity. For example, according to the methods of the invention, an anti-FRA antibody of the invention is produced by cell culture using a reference osmolarity in the lag phase and using higher osmolarity in the growth phase. The reference osmolarity may be in the ranges from 250 to 350 mOsm/L. The high osmolarity may be 360 to 800 mOsm/L, or 400 to 650 mOsm/L. In some embodiments, the cells are cultured at about 450 mOsm/L, 475 mOsm/L, 500 mOsm/L, 525 mOsm/L, 550 mOsm/L, 575 mOsm/L, 600 mOsm/L, 625 mOsm/L or 650 mOsm/L. In some embodiments, cells are cultured for a first time period at 250-350 mOsm/L and at a second time period at 600 mOsm/L. The cells may be cultured at high osmolarity from day 0 to day 14, or starting any day from 2 to day 10, preferably starting any day from day 3 to day 5, i.e., culturing the cells at a higher osmolarity, as described herein, starting at day 2, day 3, day 4 or day 5.

According to the invention, an anti-FRA antibody produced by cells cultured at high osmolarity has an N-linked neutral glycan profile comprising increased NA2, MAN5 and NGA2F and decreased NA2G1F compared to the anti-FRA antibody reference standard. According to the invention, an anti-FRA antibody produced by cells cultured at high osmolarity has an N-linked neutral glycan profile comprising increased MAN5 and/or decreased NA2G1F and/or increased NGA2F, compared to the anti-FRA antibody produced under reference cell culture conditions (Lot No. NB810-1.0). In some embodiments, the amount of the MAN5 glycoform in the anti-FRA antibody produced by cells cultured at high osmolarity may be at least 40% increased compared to the anti-FRA antibody produced under reference cell culture conditions (Lot No. NB810-10). In some embodiments, the amount of the NA2G1F glycoform in the anti-FRA antibody produced by cells cultured at high osmolarity may be at least 30% decreased compared to the anti-FRA antibody produced under reference cell culture conditions (Lot No. NB810-10). In some embodiments, the amount of the NGA2F glycoform in the anti-FRA antibody produced by cells cultured at high osmolarity may be at least 15% increased compared to the anti-FRA antibody produced under reference cell culture conditions (Lot No. NB810-10). In some embodiments, the anti-FRA antibody cultured at high osmolarity has an N-linked neutral glycan profile comprising a ratio of M3N2:M3N2F:NA2:NA2F:MAN5:NGA2:NA2G1F:NGA2F of about 1:3:3:15:43:33:250:900. In some embodiments, the anti-FRA antibody produced by cells cultured at high osmolarity has an N-linked neutral glycan profile of about 0.08% M3N2, 0.25% M3N2F, 0.23% NA2, 1.23% NA2F, 3.4% MAN5, 2.63% NGA2, 19.57% NA2G1F and 72.6% NGA2F. In some embodiments, at least 3% or 4% of the neutral glycans in the anti-FRA antibody produced by cells cultured at high osmolarity may be of the MAN5 glycoform. In some embodiments, the anti-FRA antibody produced by cells cultured at high osmolarity has an N-linked neutral glycan profile of about 6.34% non-fucosylated glycoforms and 93.65% fucosylated glycoforms.

The invention further provides methods for producing an anti-FRA antibody by culturing cells capable of expressing the antibody for at least a portion of the culture time in the presence of sodium butyrate. In some embodiments, the cells are cultured in the presence of sodium butyrate from day 0 onward, where day 0 is the day of inoculation. In other embodiments, the cells are cultured for a first time period in the absence of sodium butyrate, and for a second time period in the presence of sodium butyrate. For example, according to the methods of the invention, an anti-FRA antibody of the invention is produced by cell culture in the absence of sodium butyrate in the tag phase and in the presence of sodium butyrate in the growth phase. In some embodiments, the sodium butyrate may be used at 0.01 mM-90 mM, or 0.01 mM-16 mM concentration. In some embodiments, the sodium butyrate may be used at 0.5 mM or 10 mM concentration. In some embodiments, the sodium butyrate is added on day 6 after initiation of the culture. The cells may be cultured in the presence of sodium butyrate from day 0 to day 14, or starting any day from 2 to day 10, preferably starting from day 3, day 4, day 5, day 6 or day 7.

According to the invention, an anti-FRA, antibody produced by cells cultured in the presence of sodium butyrate has an N-linked neutral glycan profile comprising increased M3N2 and NA2 compared to the anti-FRA antibody reference standard. In some embodiments, an anti-FRA antibody produced by cells cultured in the presence of sodium butyrate has an N-linked neutral glycan profile comprising decreased MAN5, compared to the anti-FRA antibody produced under reference cell culture conditions (Lot No. NB809-65). In other embodiments, an anti-FRA antibody produced by cells cultured in the presence of sodium butyrate has an N-linked neutral glycan profile comprising decreased M3N2 and/or increased M3N2F and/or decreased NA2 and/or increased MAN5, compared to the anti-FRA antibody produced under reference cell culture conditions (Lot No. NB809-65). In some embodiments, the amount of the MAN5 glycoform in the anti-FRA antibody produced by cells cultured in the presence of sodium butyrate may be at least 25% decreased compared to the anti-FRA antibody produced under reference cell culture conditions (Lot No. NB809-65). In some embodiments, the amount of the MAN5 glycoform in the anti-FRA antibody produced by cells cultured in the presence of sodium butyrate may be at least 30% increased compared to the anti-FRA antibody produced under reference cell culture conditions (Lot No. NB809-65). In some embodiments, the amount of the M3N2 glycoform in the anti-FRA antibody produced by cells cultured in the presence of sodium butyrate may be at least 75% decreased compared to the anti-FRA antibody produced under reference cell culture conditions (Lot No. NB809-65). In some embodiments, the amount of the M3N2F glycoform in the anti-FRA antibody produced by cells cultured in the presence of sodium butyrate may be at least 70% increased compared to the anti-FRA antibody produced under reference cell culture conditions (Lot No. NB809-65). In some embodiments, the amount of the NA2 glycoform in the anti-FRA antibody produced by cells cultured in the presence of sodium butyrate may be at least 75% decreased compared to the anti-FRA antibody produced wider reference cell culture conditions (Lot No. NB809-65). In some embodiments, the anti-FRA antibody cultured in the presence of sodium butyrate has an N-linked neutral glycan profile comprising a ratio of M3N2:M3N2F:NA2:NA2F:MAN5:NGA2:NA2G1F:NGA2F of about 1:1.7:3:18:16:23:200:450. In other embodiments, the anti-FRA antibody cultured in the presence of sodium butyrate has an N-linked neutral glycan profile comprising a ratio of M3N2:M3N2F:NA2:NA2F:MAN5:NGA2:NA2G1F:NGA2F of about 1:8:3:40:80:55:500:1500. In some embodiments, the anti-FRA antibody produced by cells cultured in the presence of sodium butyrate has an N-linked neutral glycan profile of about 0.14% M3N2, 0.24% M3N2F, 0.47% NA2, 2.49% NA2F, 2.19% MAN5, 3.26% NGA2, 28.66% NA2G1F and 62.55% NGA2F. In some embodiments, the anti-FRA antibody produced by cells cultured in the presence of sodium butyrate has an N-linked neutral glycan profile of about 0.05% M3N2, 0.4% M3N2F, 0.13% NA2, 1.83% NA2F, 4.11% MAN5, 2.77% NGA2, 25.33% NA2G1F and 65.38% NGA2F. In some embodiments, at least 4% or 5% of the neutral glycans in the anti-FRA antibody produced by cells cultured in the presence of sodium butyrate may be of the MAN5 glycoform. In some embodiments, the anti-FRA antibody produced by cells cultured in the presence of sodium butyrate has an N-linked neutral glycan profile of about 6.06% non-fucosylated glycoforms and 93.94% fucosylated glycoforms. In some embodiments, the anti-FRA antibody produced by cells cultured in the presence of sodium butyrate has an N-linked neutral glycan profile of about 7.06% non-fucosylated glycoforms and 92.94% fucosylated glycoforms.

The invention further provides methods for producing an anti-FRA antibody by culturing cells capable of expressing the antibody for at least a portion of the culture time at a low dissolved oxygen tension (DO). In some embodiments, the cells are cultured at a low DO from day 0 onward, where day 0 is the day of inoculation. In other embodiments, the cells are cultured for a first time period at a reference DO, and for a second time period at a lower DO. For example, according to the methods of the invention, an anti-FRA antibody of the invention is produced by cell culture using a reference DO in the lag phase and using a lower DO in the growth phase. The reference DO may be in the range of 30-100%. For example, the DO of the reference cell culture medium may be between 30% and 40%. The lower DO may be a DO of 0%-15%, or 5%-25%, preferably 5%-20%, 5-15%, 5%-10%, such as about 5%, 6%, 7%, 8%, 9%, 10%, 11%, 12%, 13%, 14% or 15%. In some embodiments, cells are cultured for a first time period at a DO tension of 30% and at a second time period at a DO tension of 5%. The cells may be cultured at low DO starting any day from day 2 to day 10, preferably day 3 to day 7, such as starting at day 3, day 4, day 5, day 6 or day 7.

According to the invention, an anti-FRA antibody produced by cells cultured at low DO has an N-linked neutral glycan profile comprising increased NA2 and NGA2 compared to the anti-FRA antibody produced under “reference culture conditions” as defined herein. In some embodiments, an anti-FRA antibody produced by cells cultured at low DO has an N-linked neutral glycan profile comprising increased NA2 and M3N2 compared to the anti-FRA antibody produced under “reference culture conditions” as defined herein. In some embodiments, an anti-FRA antibody produced by cells cultured at low DO has an N-linked neutral glycan profile comprising decreased M3N2 and/or decreased MAN5 and/or increased NGA2 and/or increased NA2G1F and/or decreased NGA2F, compared to the anti-FRA antibody produced under reference cell culture conditions (Lot No. NB809-65). In some embodiments, an anti-FRA antibody produced by cells cultured at low DO has an N-linked neutral glycan profile comprising increased non-fucosylated glycoforms, compared to the anti-FRA antibody produced under reference cell culture conditions (Lot No. NB809-65). In some embodiments, an anti-FRA antibody produced by cells cultured at low DO has an N-linked neutral glycan profile comprising decreased M3N2, compared to the anti-FRA antibody produced under reference cell culture conditions (Lot No. NB859-25). In some embodiments, the amount of the M3N2 glycoform in the anti-FRA antibody produced by cells cultured at low DO may be at least 95% decreased compared to the anti-FRA antibody produced under reference cell culture conditions (Lot No. NB809-65). In some embodiments, the amount of the MAN5 glycoform in the anti-FRA antibody produced by cells cultured at low DO may be at least 25% decreased compared to the anti-FRA antibody produced under reference cell culture conditions (Lot No. NB809-65). In some embodiments, the amount of the NGA2 glycoform in the anti-FRA antibody produced by cells cultured at low DO may be at least 2-fold increased compared to the anti-FRA antibody produced under reference cell culture conditions (Lot No. NB809-65). In some embodiments, the amount of the non-fucosylated glycoforms in the anti-FRA antibody produced by cells cultured at low DO may be at least 40% increased compared to the anti-FRA antibody produced under reference cell culture conditions (Lot No. NB809-65). In some embodiments, the amount of the NA2G1F glycoform in the anti-FRA antibody produced by cells cultured at low DO may be at least 20% increased compared to the anti-FRA antibody produced under reference cell culture conditions (Lot No. NB809-65). In some embodiments, the amount of the NGA2F glycoform in the anti-FRA antibody produced by cells cultured at low DO may be at least 15% decreased compared to the anti-FRA antibody produced under reference cell culture conditions (Lot No. NB809-65). In some embodiments, the amount of the M3N2 glycoform in the anti-FRA antibody produced by cells cultured at low DO may be at least 80% decreased compared to the anti-FRA antibody produced under reference cell culture conditions (Lot No. NB859-25). In some embodiments, the anti-FRA antibody cultured at low DO has an N-linked neutral glycan profile comprising a ratio of M3N2:M3N2F:NA2:NA2F:MAN5:NGA2:NA2G1F:NGA2F of about 1:20:50:280:220:650:3200:5500. In other embodiments, the anti-FRA antibody cultured at low DO has an N-linked neutral glycan profile comprising a ratio of M3N2:M3N2F:NA2:NA2F:MAN5:NGA2:NA2G1F:NGA2F of about 1:8:30:40:55:80:550:1800. In some embodiments, the anti-FRA antibody produced by cells cultured at low DO has an N-linked neutral glycan profile of about 0.01% M3N2, 0.22% M3N2F, 0.48% NA2, 2.8% NA2F, 2.22% MAN5, 6.53% NGA2, 31.98% NA2G1F and 55.75% NGA2F. In some embodiments, the anti-FRA antibody produced by cells cultured at low DO has an N-linked neutral glycan profile of about 0.04% M3N2, 0.31% M3N2F, 0.3% NA2, 1.59% NA2F, 2.23% MAN5, 3.17% NGA2, 21.9% NA2G1F and 70.46% NGA2F. In some embodiments, at least 6% or 7% of the neutral glycans in the anti-FRA antibody produced by cells cultured at low DO may be of the NGA2 glycoform. In some embodiments, the anti-FRA antibody produced by cells cultured at low DO may comprise about 9% or 10% non-fucosylated glycoforms. In some embodiments, the anti-FRA antibody produced by cells cultured at low DO has an N-linked neutral glycan profile of about 9.24% non-fucosylated glycoforms and 90.75% fucosylated glycoforms. In some embodiments, the anti-FRA antibody produced by cells cultured at low DO has an N-linked neutral glycan profile of about 5.74% non-fucosylated glycoforms and 94.26% fucosylated glycoforms.

The invention also provides methods for producing an anti-FRA antibody by culturing cells capable of expressing the antibody for at least a portion of the culture time at a high CO₂ concentration. In some embodiments, the cells are cultured at a high CO₂ concentration from day 0 onward, where day 0 is the day of inoculation. Preferably, the CO₂ concentration is increased after lag phase. In other embodiments, the cells are cultured for a first time period at a reference CO₂ concentration, and for a second time period at a high CO₂ concentration. For example, according to the methods of the invention, an anti-FRA antibody of the invention is produced by cell culture using a reference CO₂ concentration in the tag phase and using high CO₂ concentration in the growth phase. The reference CO₂ concentration may be about 5%. The high CO₂ concentration can be 10%-30%, or 10%-20%, that is, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29% or about 30%. In some embodiments, cells are cultured for a first time period at a CO₂ concentration of about 5% and at a second time period at a CO₂ concentration of up to 20%. The cells may be cultured at a high CO₂ concentration starting any day from day 2 to day 10, that is, starting day 2, day 3, day 4, day 5, day 6, day 7, day 8, day 9 or day 10.

According to the invention, an anti-FRA antibody produced by cells cultured at a high CO₂ concentration has an N-linked neutral glycan profile comprising increased M3N2 and NA2 and decreased NA2F compared to the anti-FRA antibody reference standard. According to the invention, an anti-FRA antibody produced by cells cultured at a high CO₂ concentration has an N-linked neutral glycan profile comprising decreased NA2 and/or decreased MAN5, compared to the anti-FRA antibody produced under reference cell culture conditions (Lot No. NB809-65). In some embodiments, the amount of the NA2 glycoform in the anti-FRA antibody produced by cells cultured at a high CO₂ concentration may be at least 60% decreased compared to the anti-FRA antibody produced under reference cell culture conditions (Lot No. NB809-65). In some embodiments, the amount of the MAN5 glycoform. In the anti-FRA antibody produced by cells cultured at a high CO₂ concentration may be at least 40% decreased compared to the anti-FRA antibody produced under reference cell culture conditions (Lot No. NB809-65). In some embodiments, the anti-FRA antibody cultured at a high CO₂ concentration has an N-linked neutral glycan profile comprising a ratio of M3N2:M3N2F:NA2:NA2F:MAN5:NGA2:NA2G1F:NGA2F of about 1:1.5:1:7:8:16:100:400. In some embodiments, the anti-FRA antibody produced by cells cultured at a high CO₂ concentration has an N-linked neutral glycan profile of about 0.19% M3N2, 0.27% M3N2F, 0.23% NA2, 1.34% NA2F, 1.61% MAN5, 3.04% NGA2, 19.51% NA2G1F and 73.81% NGA2F. In some embodiments, the anti-FRA antibody produced by cells cultured at a high CO₂ concentration has an N-linked neutral glycan profile of about 5.07% non-fucosylated glycoforms and 94.93% fucosylated glycoforms.

The invention further provides methods for producing an anti-FRA antibody by culturing cells capable of expressing the antibody for at least a portion of the culture time in the presence of copper chloride (CuCl). In some embodiments, the cells are cultured in the presence of CuCl from day 0 onward, where day 0 is the day of inoculation. In other embodiments, the cells are cultured for a first time period in the absence of CuCl, and for a second time period in the presence of CuCl. For example, according to the methods of the invention, an anti-FRA antibody of the invention is produced by cell culture in the absence of CuCl in the lag phase and in the presence of CuCl in the growth phase. The CuCl may be used at any concentration 0.01-0.5 mM, or 0.01 mM-0.5 mM. In some embodiments, the CuCl is used at 0.5 mM concentration. In some embodiments, the CuCl may be added starting any day from day 0 to day 14 after initiation of the culture. In some embodiments, the CuCl is added on day 6 after initiation of the culture.

According to the invention, an anti-FRA antibody produced by cells cultured in the presence of CuCl has an N-linked neutral glycan profile comprising increased M3N2, M3N2F and NA2 compared to the anti-FRA antibody reference standard. According to the invention, an anti-FRA antibody produced by cells cultured in the presence of CuCl has an N-linked neutral glycan profile comprising increased M3N2 and/or increased M3N2F and/or decreased NA2 and/or decreased MAN5 and/or increased NA2G1F and/or decreased NGA2F, compared to the anti-FRA antibody produced under reference cell culture conditions (Lot No. NB809-65). In some embodiments, the amount of the M3N2 glycoform in the anti-FRA antibody produced by cells cultured in the presence of CuCl may be at least 50% increased compared to the anti-FRA antibody produced under reference cell culture conditions (Lot No. NB809-65). In some embodiments, the amount of the M3N2F glycoform in the anti-FRA antibody produced by cells cultured in the presence of CuCl may be at least 60% increased compared to the anti-FRA antibody produced under reference cell culture conditions (Lot No. NB809-65). In some embodiments, the amount of the NA2 glycoform in the anti-FRA antibody produced by cells cultured in the presence of CuCl may be at least 75% decreased compared to the anti-FRA antibody produced under reference cell culture conditions (Lot No. NB809-65). In some embodiments, the amount of the MAN5 glycoform in the anti-FRA antibody produced by cells cultured in the presence of CuCl may be at least 25% decreased compared to the anti-FRA antibody produced under reference cell culture conditions (Lot No. NB809-65). In some embodiments, the amount of the NA2G1F glycoform in the anti-FRA antibody produced by cells cultured in the presence of CuCl may be at least 30% increased compared to the anti-FRA antibody produced under reference cell culture conditions (Lot No. NB809-65). In some embodiments, the amount of the NGA2F glycoform in the anti-FRA antibody produced by cells cultured in the presence of CuCl may be at least 10% decreased compared to the anti-FRA antibody produced under reference cell culture conditions (Lot No. NB809-65). In some embodiments, the anti-FRA antibody cultured in the presence of CuCl has an N-linked neutral glycan profile comprising a ratio of M3N2:M3N2F:NA2:NA2F:MAN5:NGA2:NA2G1F:NGA2F of about 2:2:1:17:14:25:220:400. In some embodiments, the anti-FRA antibody produced by cells cultured in the presence of CuCl has an N-linked neutral glycan profile of about 0.34% M3N2, 0.37% M3N2F, 0.15% NA2, 2.6% NA2F, 2.16% MAN5, 3.88% NGA2, 33.03% NA2G1F and 57.46% NGA2F. In some embodiments, the anti-FRA antibody produced by cells cultured in the presence of CuCl has an N-linked neutral glycan (profile of about 6.53% non-fucosylated glycoforms and 93.46% fucosylated glycoforms.

According to the invention, the anti-FRA antibody may be harvested at about 10 days (240 hours), 13 days, 14 days (336 hours), 15 days, 17 days (408 hours) or 20 days (480 hours) after initiation of the cell culture.

According to the invention, an anti-FRA antibody produced by cells harvested at 10 days has an N-linked neutral glycan profile comprising increased M3N2 and NA2 compared to the anti-FRA antibody reference standard. According to the invention, an anti-FRA antibody produced by cells harvested at 17 days has an N-linked neutral glycan profile comprising increased M3N2, NA2 and MAN5 compared to the anti-FRA antibody produced under “reference culture conditions” as defined herein. According to the invention, an anti-FRA antibody produced by cells harvested at 20 days has an N-linked neutral glycan profile comprising increased M3N2 and NA2 compared to the anti-FRA antibody produced under “reference culture conditions” as defined herein.

According to the invention, an anti-FRA antibody produced by cells harvested at 10 days has an N-linked neutral glycan profile comprising decreased M3N2 and/or decreased M3N2F and/or decreased MAN5, compared to the anti-FRA antibody produced under reference cell culture conditions (Lot No. NB859-25). In some embodiments, an anti-FRA antibody produced by cells harvested at 10 days has an N-linked neutral glycan profile comprising decreased non-fucosylated glycoforms, compared to the anti-FRA antibody produced under reference cell culture conditions (Lot No. NB859-25). In some embodiments, the amount of the M3N2 glycoform in the anti-FRA antibody produced by cells harvested at 10 days may be at least 80% decreased compared to the anti-FRA antibody produced under reference cell culture conditions (Lot No. NB859-25). In some embodiments, the amount of the M3N2F glycoform in the anti-FRA antibody produced by cells harvested at 10 days may be at least 25% decreased compared to the anti-FRA antibody produced under reference cell culture conditions (Lot No. NB859-25). In some embodiments, the amount of the MAN5 glycoform in the anti-FRA antibody produced by cells harvested at 10 days may be at least 50% decreased compared to the anti-FRA antibody produced under reference cell culture conditions (Lot No. NB859-25). In some embodiments, the amount of the non-fucosylated glycoforms in the anti-FRA antibody produced by cells harvested at 10 days may be at least 23% decreased compared to the anti-FRA antibody produced under reference cell culture conditions (Lot No. NB859-25). In some embodiments, the anti-FRA antibody harvested at 10 days has an N-linked neutral glycan profile comprising a ratio of M3N2:M3N2F:NA2:NA2F:MAN5:NGA2:NA2G1F:NGA2F of about 1:6:8:40:30:70:550:1800. In some embodiments, the anti-FRA antibody harvested at 10 days has an N-linked neutral glycan profile of about 0.04% M3N2, 0.24% M3N2F, 0.31% NA2, 1.48% NA2F, 1.28% MAN5, 2.64% NGA2, 22.33% NA2G1F and 71.68% NGA2F. In some embodiments, the anti-FRA antibody harvested at 10 days has an N-linked neutral glycan profile of about 4.27% non-fucosylated glycoforms and 95.73% fucosylated glycoforms.

According to the invention, an anti-FRA antibody produced by cells harvested at 17 days has an N-linked neutral glycan profile comprising increased NA2, compared to the anti-FRA antibody produced under reference cell culture conditions (Lot No. NB859-25). In some embodiments, the amount of the NA2 glycoform in the anti-FRA antibody produced by cells harvested at 17 days may be at least 50% increased compared to the anti-FRA antibody produced under reference cell culture conditions (Lot No. NB859-25). In some embodiments, the anti-FRA antibody harvested at 17 days has an N-linked neutral glycan profile comprising a ratio of M3N2:M3N2F:NA2:NA2F:MAN5:NGA2:NA2G1F:NGA2F of about 1:2:3:13:20:18:170:440. In some embodiments, the anti-FRA antibody harvested at 17 days has an N-linked neutral glycan profile of about 0.15% M3N2, 0.34% M3N2F, 0.51% NA2, 1.9% NA2F, 3.16% MAN5, 2.77% NGA2, 24.87% NA2G1F and 66.3% NGA2F. In some embodiments, at least 3% or 4% of the neutral glycans in the anti-FRA antibody produced by cells harvested at day 17 may be of the MAN5 glycoform. In some embodiments, the anti-FRA antibody harvested at 17 days has an N-linked neutral glycan profile of about 6.59% non-fucosylated glycoforms and 93.41% fucosylated glycoforms.

In some embodiments, the anti-FRA antibody harvested at 20 days has an N-linked neutral glycan profile comprising a ratio of M3N2:M3N2F:NA2:NA2F:MAN5:NGA2:NA2G1F:NGA2F of about 1:1.8:2.5:10:19:17:140:430. In some embodiments, the anti-FRA antibody harvested at 20 days has an N-linked neutral glycan profile of about 0.16% M3N2, 0.29% M3N2F, 0.4% NA2, 1.66% NA2F, 2.96% MAN5, 2.64% NGA2, 22.43% NA2G1F and 69.45% NGA2F. In some embodiments, the anti-FRA antibody harvested at 20 days has an N-linked neutral glycan profile of about 6.16% non-fucosylated glycoforms and 93.83% fucosylated glycoforms.

The invention encompasses a method of producing an anti-FRA antibody using any one or more of the conditions described above.

In one embodiment, the anti-FRA antibody of the invention has altered binding affinity for human FRA relative to the anti-FRA reference standard. Binding affinity in some embodiments may be reduced compared to a MORAb-003 reference standard, and reduced compared to the antibody produced under reference culture conditions as defined herein (positive control). Such antibodies may be produced by culturing host cells expressing the anti-FRA antibody: using galactose as a sugar source, at tow temperature, at high osmolarity, in the presence of sodium butyrate, at low DO, at a high CO₂ concentration, in the presence of copper chloride, or by harvesting cells expressing the anti-FRA antibody 10, 17 or 20 days after the initiation of the culture, as described herein. In embodiments using galactose as a sugar source, 2 g/L of galactose may be added at 5 days after initiation of the culture. In embodiments using low temperature, the culture temperature may be shifted from 36.5° C. to 30° C. at 5 days after initiation of the culture. In embodiments using increased osmolarity, the osmolarity of the culture may be increased to 600 mOsm/L by adding NaCl at 7 days after initiation of the culture. In embodiments using reduced dissolved oxygen tension (DO), the DO may be changed from 30% to 5% at 6 days after initiation of the culture. In embodiments using sodium butyrate, 0.5 mM or 10 mM sodium butyrate may be added at 6 days after initiation of the culture. In embodiments using copper chloride, 0.5 mM copper chloride may be added at 6 days after initiation of the culture. In other embodiments, the CO₂ concentration in the medium was increased to 20% at 5 days after initiation of the culture.

In any of the foregoing embodiments, the culture may consist of CHO cells, for example CHO-K1 cells comprising nucleic acids encoding the heavy chain amino acid sequence of SEQ ID NO: 1 or SEQ ID NO: 5 or encoding a sequence that is at least 95% identical, and the light chain amino acid sequence of SEQ ID NO: 2 or SEQ ID NO: 6 or SEQ ID NO: 7 or encoding a sequence that is at least 95% identical. In particular, the cell may comprise nucleic acids encoding the heavy chain amino acid sequence of SEQ ID NO: 1 and a light chain amino acid sequence that is 99% identical to the light chain amino acid sequence of SEQ ID NO: 2, the heavy chain amino acid sequence of SEQ ID NO: 1 and the light chain amino acid sequence of SEQ ID NO: 6, or the heavy chain amino acid sequence of SEQ ID NO: 5 and the light chain amino acid sequence of SEQ ID NO: 7, or encoding sequences that are at least 95% identical to the above-mentioned sequences. In some embodiments, the nucleic acid encoding the heavy chain comprises the nucleotide sequence of SEQ ID NO: 8 with or without the nucleotides encoding the leader sequence, and the nucleic acid encoding the light chain comprises the nucleotide sequence of SEQ ID NO: 9 with or without the nucleotides encoding the leader sequence. In various embodiments the anti-FRA antibody comprises the heavy chain amino acid sequence of SEQ ID NO: 1 or SEQ ID NO: 5 or a sequence that is at least 95% identical, and the light chain amino acid sequence of SEQ ID NO: 2 or SEQ ID NO: 6 or SEQ ID NO: 7 or a sequence that is at least 95% identical. In particular embodiments, the antibody comprises the heavy chain amino acid sequence of SEQ ID NO: 1 and a light chain amino acid sequence that is 99% identical to the light chain amino acid sequence of SEQ ID NO: 2, the heavy chain amino acid sequence of SEQ ID NO: 1 and the light chain amino acid sequence of SEQ ID NO: 6, or the heavy chain amino acid sequence of SEQ ID NO: 5 and the light chain amino acid sequence of SEQ ID NO: 7, or sequences that are at least 95% identical to the above-mentioned sequences.

The binding affinity of the anti-FRA antibody can be measured by ELISAs, RIAs, flow cytometry or surface plasmon resonance, such as BIACORE®. The term “surface plasmon resonance,” as used herein, refers to an optical phenomenon that allows for the analysis of real-time biospecific interactions by detection of alterations in protein concentrations within a biosensor matrix, for example using the BIACORE® system (Pharmacia Biosensor AB, Uppsala, Sweden and Piscataway, N.J.). For further descriptions, see Jonsson U. et al., Ann. Biol. Clin. 51:19-26 (1993); Jonsson U. et al., Biotechniques 11:620-627 (1991); Jonsson B. et al., J. Mol. Recognit. 8:125-131 (1995); and Johnsson B. et al., Anal. Biochem. 198:268-277 (1991).

The binding affinity of the anti-FRA antibody may be reduced at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% relative to an anti-FRA antibody produced under “reference culture conditions” as defined herein. In various embodiments, the anti-FRA antibody of the invention with reduced binding to human FRA comprises the heavy chain amino acid sequence of SEQ ID NO: 1 or SEQ ID NO: 5 or a sequence that is at least 95% identical, and the light chain amino acid sequence of SEQ ID NO: 2 or SEQ ID NO: 6 or SEQ ID NO: 7 or a sequence that is at least 95% identical. In particular, the antibody comprises the heavy chain amino acid sequence of SEQ ID NO: 1 and a light chain amino acid sequence that is 99% identical to the light chain amino acid sequence of SEQ ID NO: 2, the heavy chain amino acid sequence of SEQ ID NO: 1 and the light chain amino acid sequence of SEQ ID NO: 6, or the heavy chain amino acid sequence of SEQ ID NO: 5 and the light chain amino acid sequence of SEQ ID NO: 7, or sequences that are at least 95% identical to the above-mentioned sequences.

An anti-FRA monoclonal antibody with reduced binding affinity for human FRA is useful for treating cancer. Without being bound by theory, the reduced binding affinity may allow deeper penetration into tumors, to allow targeting of inner tumor mass. See, Adams et al, “High Affinity Restricts the Localization and Tumor Penetration of Single-Chain Fv Antibody Molecules,” Cancer Res 61:4750-4755 (Jun. 15, 2001).

In some embodiments, the anti-FRA antibody of the invention may have altered antibody-dependent cellular cytotoxicity (ADCC) relative to an anti-FRA antibody produced under “reference culture conditions” as defined herein. In some embodiments, the ADCC is increased compared to the anti-FRA antibody reference standard. In some embodiments, an antibody produced by culturing under an altered culture condition also has increased ADCC compared to the antibody produced under reference conditions (positive control). In other embodiments, the ADCC is increased compared to a positive control but reduced compared to the antibody reference standard. In still other embodiments, the ADCC is reduced compared to a positive control and to the antibody reference standard. Anti-FRA monoclonal antibodies with increased ADCC are useful for therapies where the mode of action is to elicit immune effector functions against FRA-expressing target cells, for example, to treat diseases and conditions where it is desired to kill FRA-expressing cells. Anti-FRA monoclonal antibodies with reduced ADCC are useful in therapies where the mode of action is to use the antibody as a targeting agent to deliver a cytotoxic payload to target cell. Without being bound by theory, it may be desirable to reduce ADCC activity to maximize the local concentration of the antibody conjugate, as well as the internalization efficiency of the antibody. Interaction of the antibody with immune effector cells, as would be necessary for ADCC, would be predicted to reduce the availability of the antibody conjugate at the cell surface, and decrease the efficacy of the antibody conjugate.

As used herein, ADCC is an immune effector activity of the antibody. ADCC may be mediated by Fc receptors on effector cells, which include but are not limited to cytotoxic cells, natural killer (NK) cells, or macrophages, leading to cell lysis and/or death of the FRA-expressing target cells. ADCC of an anti-FRA antibody of the invention may be measured using standard assays known in the art (see, e.g., U.S. Patent Application Publication No. 2006/0239911, which is incorporated by reference in its entirety). For example, an FRA-expressing cell may be exposed to various concentrations of an anti-FRA antibody (or negative controls such as no antibody or control Ig) and activated effector cells, such as peripheral blood mononuclear cells (PBMCs). ADCC may be monitored by lactate dehydrogenase (LDH) release that occurs upon cell lysis of the FRA-expressing cells. The activity of LDH may be measured by a spectrophotometric assay. ADCC may also be measured by labeling FRA-expressing cells with carboxyfluorescein diacetate succinimidyl ester (CFDA SE). Labeled cells are then mixed with dilutions of the anti-FRA antibody and unlabeled effector cells derived from PBMCs. After incubation, the cell populations are scored by flow cytometry for remaining viable, labeled FRA-expressing cells.

In various embodiments, an anti-FRA antibody of the invention may elicit ADCC that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% higher than that occurring relative to an anti-FRA antibody produced under “reference culture conditions” (i.e., antibody reference standard and/or positive control) as defined herein. In other embodiments, an anti-FRA antibody of the invention may elicit ADCC that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% tower than that occurring relative to an anti-FRA antibody produced under “reference culture conditions” as defined herein.

According to methods of the invention, ADCC of an anti-FRA antibody may be increased by culturing host cells expressing the antibody at low temperature or at low DO as described herein. In some embodiments, the low temperature may be a shift from 36.5° C. to 30° C. at 5 days after initiation of the culture. In some embodiments, the DO may be changed from 30% to 5% at 6 days after initiation of the culture. According to other methods, the ADCC may be decreased by harvesting the antibody from the culture earlier than 13 days or later than 15 days from initiation of culture.

In any of the foregoing embodiments, the culture may consist of CHO cells, for example CHO-K1 cells comprising nucleic acids encoding the heavy chain amino acid sequence of SEQ ID NO: 1 or SEQ ID NO: 5 or encoding a sequence that is at least 95% identical, and the light chain amino acid sequence of SEQ ID NO: 2 or SEQ ID NO: 6 or SEQ ID NO: 7 or encoding a sequence that is at least 95% identical. In particular, the cell may comprise nucleic acids encoding the heavy chain amino acid sequence of SEQ ID NO: 1 and a light chain amino acid sequence that is 99% identical to the light chain amino acid sequence of SEQ ID NO: 2, the heavy chain amino acid sequence of SEQ ID NO: 1 and the light chain amino acid sequence of SEQ ID NO: 6, or the heavy chain amino acid sequence of SEQ ID NO: 5 and the tight chain amino acid sequence of SEQ ID NO: 7, or encoding sequences that are at least 95% identical to the above-mentioned sequences. In some embodiments, the nucleic acid encoding the heavy chain comprises the nucleotide sequence of SEQ ID NO: 8 with or without the nucleotides encoding the leader sequence, and the nucleic acid encoding the light chain comprises the nucleotide sequence of SEQ ID NO: 9 with or without the nucleotides encoding the leader sequence.

In accordance with the invention, increased ADCC correlates with an increased amount of NGA2 (G0) glycans or non-glycosylated glycans in the anti-FRA antibody and is inversely correlated with the amount of M3N2F glycans in the anti-FRA antibody.

In some embodiments, an anti-FRA antibody of the invention internalizes in a cell upon binding to FRA on the cell surface. Such internalizing antibodies may be conjugated to chemotherapeutic agents, such as immunotoxins, radionuclides, or cytotoxic and cytostatic agents. In one embodiment, the anti-FRA antibody of the invention may have altered internalization efficiency or internalization rate relative to an antibody produced under reference culture conditions. In this context, internalization efficiency refers to the ability with which the anti-FRA antibody can be retained inside a target cell, while the internalization rate refer to the rate at which an anti-FRA antibody can traverse the cell membrane of a target cell. Standard assays known in the art may be used to monitor internalization of an anti-FRA antibody of the invention in FRA-expressing cells (see, e.g., U.S. Patent Application Publication No. 2006/0239911, which is incorporated by reference in its entirety). For example, second immunotoxins, such as the Hum-ZAP assay (Advanced Targeting Systems, San Diego, Calif., USA), may be used to monitor internalization of anti-FRA antibody of the invention. Second immunotoxins are conjugates of a secondary antibody, such as a goat anti-human IgG, and the ribosome-inactivating protein, saporin. Such second immunotoxins may be selected so that they bind to an anti-FRA antibody of the invention. If the anti-FRA antibody is internalized, the saporin will inhibit protein synthesis and cause cell death. The cell viability of FRA-expressing cells exposed to an anti-FRA antibody of the invention and a second immunotoxin (or negative controls) may be measured with standard cell viability assays, such as those that read viable cell number by spectrophotometry.

in some embodiments, an anti-ETA antibody of the invention may exhibit an internalization rate that is at least 5%, at least 10%, at least 20%, at least 30%, at least 40%, at least 50%, at least 60%, at least 70%, at least 80%, at least 90%, at least 99%, or at least 99.9% higher than an anti-FRA antibody produced under “reference culture conditions” as defined herein. An anti-FRA monoclonal antibody with an increased internalization rate is useful for example for antibody conjugates, where faster internalization may equate to better pharmacodynamics, and potentially less frequent or lower dosing. Additionally, for therapies in which it is desired to remove the antigen from the cell surface by internalization of the antibody antigen complex, a faster internalization rate would be expected to lead to increased efficiency in removal of the antigen from the cell surface. For example, in therapies targeting FRA-mediated signaling pathways involved in cancer cell growth, the anti-FRA antibody reduces receptor signaling by removing the receptor from the cell surface by internalization.

In other embodiments, an anti-FRA antibody of the invention exhibits an internalization rate that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% lower than an anti-FRA antibody produced under “reference culture conditions” as defined herein.

An anti-FRA monoclonal antibody with a reduced internalization rate may be useful when the therapeutic mode of action involves both intracellular mechanisms and effector function. Without being bound by theory, an anti-FRA antibody that internalizes more slowly remains at the cell surface and has increased opportunity for effector activity, including ADCC and CDC. In some embodiments, an anti-FRA antibody of the invention may have a half maximal internalization constant of about 57 minutes or 58 minutes. In some embodiments, the anti-FRA antibody of the invention has a half-maximal internalization constant of about 36 minutes, 37 minutes, 41 minutes, 42 minutes, 45 minutes or 46 minutes. The half-maximal internalization constant may be determined by may be determined by the internalization assays described herein, including FACS analysis.

In one embodiment, an anti-FRA antibody of the invention may exhibit reduced internalization efficiency compared to the internalization efficiency of an anti-FRA antibody produced under “reference culture conditions” as defined herein. An anti-FRA antibody with reduced internalization efficiency is useful to increase the availability of the antibody to the immune system, and further potentiate ADCC or complement-dependent cytotoxicity (CDC). In some embodiments, an anti-FRA antibody of the invention may exhibit an internalization efficiency that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% lower than an anti-FRA antibody produced under “reference culture conditions” as defined herein. In some embodiments, an anti-FRA antibody of the invention may have an internalization IC50 of 1632 ng/ml, or 990 mg/mL. In some embodiments, the anti-FRA antibody of the invention has an internalization IC50 of 1632 ng/mL or 990 ng/mL. The IC50 and EC50 may be determined by the internalization assays described herein.

The internalization rate may be increased by culturing host cells expressing the anti-FRA antibody at low DO or by harvesting the anti-FRA antibody 17 or 20 days after initiation of the culture as described herein. In some embodiments, the DO may be changed from 30% to 5% at 6 days after initiation of the culture.

In any of the foregoing embodiments, the culture may consist of CHO cells, for example CHO-K1 cells comprising nucleic acids encoding the heavy chain amino acid sequence of SEQ ID NO: 1 or SEQ ID NO: 5 or encoding a sequence that is at least 95% identical, and the light chain amino acid sequence of SEQ ID NO: 2 or SEQ ID NO: 6 or SEQ ID NO: 7 or encoding a sequence that is at least 95% identical. In particular, the cell may comprise nucleic acids encoding the heavy chain amino acid sequence of SEQ ID NO: 1 and a light chain amino acid sequence that is 99% identical to the light chain amino acid sequence of SEQ ID NO: 2, the heavy chain amino acid sequence of SEQ ID NO: 1 and the light chain amino acid sequence of SEQ ID NO: 6, or the heavy chain amino acid sequence of SEQ ID NO: 5 and the light chain amino acid sequence of SEQ ID NO: 7, or encoding sequences that are at least 95% identical to the above-mentioned sequences. In some embodiments, the nucleic acid encoding the heavy chain comprises the nucleotide sequence of SEQ ID NO: 8 with or without the nucleotides encoding the leader sequence, and the nucleic acid encoding the light chain comprises the nucleotide sequence of SEQ ID NO: 9 with or without the nucleotides encoding the leader sequence.

### Pharmaceutical Compositions

In a further aspect, the invention provides a composition that comprises an anti-FRA antibody of the invention, particularly the MORAb-003 antibody, with one or more of the altered features described herein and a pharmaceutically acceptable carrier or vehicle. A “pharmaceutically acceptable carrier” may be a solvent, dispersion media, coatings, antibacterial and antifungal agents, isotonic and absorption delaying agents, and the like that are physiologically compatible. Some examples of pharmaceutically acceptable carriers, merely by way of illustration, are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

A composition comprising the anti-FRA antibody of this invention may be in any suitable form for administration to a subject, for example, liquid, semi-solid and solid dosage forms, such as liquid solutions (e.g., injectable and infusible solutions), dispersions or suspensions, aerosols, tablets, pills, powders, liposomes and suppositories. As a skilled worker would appreciate, the form depends on the intended mode of administration and therapeutic application. In some embodiments, an anti-FRA composition of the invention is in the form of injectable or infusible solutions, for example the compositions may be similar to those used for passive immunization of humans. The preferred mode of administration is parenteral intravenous, subcutaneous, intraperitoneal, intramuscular) such as by intravenous infusion or injection but administration by intramuscular or subcutaneous injection, oral and nasal routes also is contemplated. Other modes of administration contemplated by the invention include intrabronchial, transmucosal, intraspinal, intrasynovial, intraaortic, ocular, otic, topical and buccal, and intratumoral.

In some embodiments, the anti-FRA antibody compositions for therapeutic use are sterile and stable under the conditions of manufacture and storage. The invention includes compositions formulated as a solution, microemulsion, dispersion, liposome, or other ordered structure suitable to high drug concentration. Sterile injectable solutions of the invention can be prepared by incorporating the anti-FRA antibody in the required amount in an appropriate solvent with one or a combination of ingredients enumerated above, as required, followed by filtered sterilization. Dispersions comprising an anti-FRA antibody of the invention may be prepared by incorporating the active compound into a sterile vehicle that contains a basic dispersion medium and the required other ingredients from those enumerated above. In the case of sterile powders for the preparation of sterile injectable solutions comprising an anti-FRA antibody of the invention, the preferred methods of preparation are vacuum drying and freeze-drying that yields a powder of the active ingredient plus any additional desired ingredient from a previously sterile-filtered solution thereof. The proper fluidity of the solution can be maintained, for example, by the use of a coating such as lecithin, by the maintenance of the required particle size in the case of dispersion and by the use of surfactants. Those of skill in the art will appreciate that to prolong absorption of injectable compositions of the invention, one can include in the composition an agent that delays absorption, for example, monostearate salts and gelatin.

In certain embodiments, an anti-FRA antibody of the invention may be prepared with a carrier that wilt protect the antibody against rapid release, as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are generally known to those skilled in the art. See, e.g., Sustained and Controlled Release Drug Delivery Systems (J, R. Robinson, ed., Marcel Dekker, Inc., New York, 1978).

In some embodiments, compositions comprising an anti-FRA antibody of the invention further comprise additional active compounds. In certain embodiments, an anti-FRA antibody of the invention is co-formulated with and/or co-administered with one or more additional therapeutic, diagnostic, or prophylactic agents. Therapeutic agents include, without limitation, an anti-FRA antibody with a different fine specificity, antibodies that bind other targets, nonsteroidal anti-inflammatory agents, analgesic agents, anticancer agents, steroids, anti-allergy agents, chemotherapeutic agents, antineoplastic agents and cytotoxic agents.

According to the invention, an anti-FRA antibody of the invention may be co-formulated with an antibody or other agent that is known to inhibit tumor or cancer cell proliferation, e.g., an antibody or agent that inhibits erbB2 receptor, E-selectin, CD20, VEGF (for example, AVASTIN® (bevacizumab), LUCENTIS® (ranibizumab) and MACUGEN® (pegaptanib)), VEGF receptor 1 (VEGFR1), VEGF receptor 2 (VEGFR2) or VEGF receptor 3 (VEGFR3).

Examples of chemotherapeutic agents include, without limitation, GLEEVEC® (imatinib), ERBITUX® (cetuximab), L-asparaginase, IRESSA® (gefitinib), TARCEVA® (erlotinib) and VELCADE® (bortezomib) and the like.

More specifically, the anti-FRA antibody of the invention may be co-formulated with alkylating agents. Examples of useful alkylating agents include, without limitation, altretamine (hexamethylmelamine), busulfan, carboplatin, carmustine (BCNU), chlorambucil, cisplatin, CYTOXAN® (cyclophosphamide), dacarbazine (DTIC), ifosfamide, lomustine, mechlorethamine (nitrogen mustard), melphalan, oxalaplatin, streptozocin, TEMODAR® (temozolomide) and thiotepa and the like.

The anti-FRA antibody of the invention may be co-formulated with antimetabolites. Examples of useful antimetabolites include, without limitation, 5-fluorouracil (5-FU), 6-mercaptopurine (6-MP), XELODA® (capecitabine), ARA-C® (cytarabine), fludarabine, GEMZAR® (gemcitabine), methotrexate and ALIMTA® (pemetrexed) and the like.

The anti-FRA antibody of the invention may be co-formulated with topoisomerase I and II inhibitors, including, without limitation, CAMPTOSAR® (irinotecan HCl), SN-38, camptothecin, HYCAMTIN® (topotecan), etoposide, teniposide, ELLENCE® (epirubicin), ADRIAMYCIN® (doxorubicin), idarubicin, mitoxantrone, lamellarin D and HU-331 (Kogan et al. (2007) Molecular Cancer Therapeutics 6:173-183, incorporated herein by reference) and the like.

In some embodiments, the anti-FRA antibody of the invention may be co-formulated with anti-tumor antibiotics, such as actinomycin-D, bleomycin, and mitomycin-C and the like.

In some embodiments, the anti-FRA antibody of the invention may be co-formulated with mitotic inhibitors. Non-limiting examples of useful mitotic inhibitors include EMCYT® (estramustine), IXEMPRA® (ixabepilone), TAXOTERE® (docetaxel), TAXOL® (paclitaxel), VELBAN® (vinblastine), ONCOVIN® (vincristine), and NAVELBINE® (vinorelbine) and the like.

In some embodiments, the anti-FRA antibody of the invention may be co-formulated with differentiating agents. Non-limiting examples of useful differentiating agents include arsenic trioxide, retinoids, tretinoin and TARGRETIN® (bexarotene) and the like.

In some embodiments, the anti-FRA antibody of the invention may be co-formulated with steroid compounds, such as, for example, prednisone, methylprednisolone and dexamethasone and the like.

In some embodiments, the anti-FRA antibody of the invention may be co-formulated with hormone-related compounds. Non-limiting examples of useful hormone-related compounds include estrogens, progestins (such as MEGACE® (megestrol acetate)), FASLODEX® (fulvestrant), tamoxifen, toremifene, LUPRON® (leuprolide), ZOLADEX® (goserelin), ARIMIDEX® (anastrozole), FEMARA® (letrozole), AROMASIN® (exemestane), CASIODEX® (bicalutamide), EULEXIN® (flutamide) and NILANDRON® (nilutamide).

In some embodiments, the anti-FRA antibody of the invention may be co-formulated with COX-II (cyclooxygenase II) inhibitors. Non-limiting examples of useful COX-II inhibitors include CELEBREX® (celecoxib), valdecoxib, and rofecoxib and the like.

In some embodiments, the anti-FRA antibody of the invention may be co-formulated with immunotherapeutic agents. Non-limiting examples of useful immunotherapeutic agents include the interferons (such as interferon-alpha), BCG, interleukin-2 (IL-2), thalidomide, lenalidomide, CAMPATH® (alemtuzumab) and RITUXAN® (rituximab) and the like.

In some embodiments, the anti-FRA antibody of the invention may be co-formulated with an MMP inhibitor. For example, the anti-ETA antibody may be co-formulated with anti-angiogenic agents, such as MMP-2 (matrix-metalloproteinase 2) inhibitors or WIMP-9 (matrix-metalloproteinask 9) inhibitors. Preferred MMP inhibitors are those that do not demonstrate arthralgia. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13). Some specific examples of MMP inhibitors useful in the present invention are AG-3340, RO 32-3555, RS 13-0830, and the compounds recited in the following list: 3-[[4-(4-fluoro-phenoxy)-benzenesulfonyl]-(1-hydroxycarbamoyl-cyclopentyl)-amino]-propionic acid; 3-exo-3-[4-(4-fluoro-phenoxy)-benzenesulfonylamino]-8-oxa-bicyclo[3.2.1]octane-3-carboxylic acid hydroxyamide; (2R,3R) 1-[4-(2-chloro-4-fluoro-benzyloxy)-benzenesulfonyl-]-3-hydroxy-3-methyl-piperidine-2-carboxylic acid hydroxyamide; 4-[4-(4-fluoro-phenoxy)-benzenesulfonylamino]-tetrahydro-pyran-4-carboxylic acid hydroxyamide; 3-[[4-(4-fluoro-phenoxy)-benzenesulfonyl]-(1-hydroxy-carbamoyl-cyclobutyl)-amino]-propionic acid; 4-[4-(4-chloro-phenoxy)-benzenesulfonylamino]-tetrahydro-pyran-4-carboxylic acid hydroxyamide; (R) 3-[4-(4-chloro-phenoxy)-benzenesulfonylamino]-tetrahydro-pyran-3-carboxylic acid hydroxyamide; (2R,3R) 1-[4-(4-fluoro-2-methyl-benzyloxy)-benzenesulfonyl]-3-hydroxy-3-methyl-piperidine-2-carboxylic acid hydroxyamide; 3-[[4-(4-fluoro-phenoxy)-benzenesulfonyl]-(1-hydroxycarbamoyl-1-methyl-ethyl)-amino]-propionic acid; 3-[[4-(4-fluoro-phenoxy)-benzenesulfonyl]-(4-hydroxycarbamoyl-tetrahydro-pyran-4-yl)-amino]-propionic acid; 3-exo-3-[4-(4-chloro-phenoxy)-benzenesulfonylamino]-8-oxa-icyclo[3.2.1]octane-3-carboxylic acid hydroxyamide; 3-endo-3-[4-(4-fluoro-phenoxy)-benzenesulfonylamino]-8-oxa-icyclo[3.2.1]octane-3-carboxylic acid hydroxyamide; and (R) 3-[4-(4-fluoro-phenoxy)-benzenesulfonylamino]-tetrahydro-furan-3-carboxylic acid hydroxyamide and the like; and pharmaceutically acceptable salts and solvates of said compounds.

In some embodiments, the anti-FRA antibody may be co-formulated with an integrin inhibitor. Integrin inhibitors, include, without limitation, obtustatin, rhodocetin, Vitaxin (MedImmune), cilengitide (EMD 121974; Merck), S137 (Pfizer), S247 (Pfizer) and JSM6427 (Jerini) (see, e.g., Brown et al. (2008) International Journal of Cancer 123: 2195-2203; Stupp et al. (2007) Journal of Clinical Oncology 25: 1637-1638; Eble et al, (2003) Biochem J. 376: 77-85, all incorporated herein by reference).

The compositions of the invention may include a “therapeutically effective amount” or a “prophylactically effective amount” of an anti-FRA antibody of the invention. A “therapeutically effective amount” refers to an amount effective, at dosages and for periods of time necessary, to achieve the desired therapeutic result. A therapeutically effective amount of the antibody or antibody portion may vary according to factors such as the disease state, age, sex, and weight of the individual, and the ability of the antibody or antibody portion to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount.

Dosage regimens can be adjusted to provide the optimum desired response (e.g., a therapeutic or prophylactic response). For example, a single bolus can be administered, several divided doses can be administered over time or the dose can be proportionally reduced or increased as indicated by the exigencies of the therapeutic situation is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the anti-FRA antibody and the particular therapeutic or prophylactic effect to be achieved, and (b) the limitations inherent in the art of compounding such an antibody for the treatment of sensitivity in individuals.

An exemplary, non-limiting range for a therapeutically or prophylactically-effective amount of anti-FRA antibody of the invention is 0.025 to 50 mg/kg, 0.1 to 50 mg/kg, 0.1-25 mg/kg, 0.1 to 10 mg/kg or 0.1 to 3 mg/kg. In one embodiment, the anti-FRA antibody is administered in a formulation as a sterile aqueous solution having a pH that ranges from about 5.0 to about 6.5 and comprising from about 1 mg/ml to about 200 mg/ml of the antibody, from about 1 millimolar to about 100 millimolar of Tween, from about 0.01 mg/ml to about 10 mg/ml of polysorbate 80 or polysorbate 20, from about 100 millimolar to about 400 millimolar of a non-reducing sugar selected from but not limited to trehalose or sucrose, from about 0.01 millimolar to about 1.0 millimolar of disodium EDTA dihydrate and optionally comprise a pharmaceutically acceptable antioxidant in addition to a chelating agent. Suitable antioxidants include, but are not limited to, methionine, sodium thiosulfate, catalase, and platinum. For example, the composition may contain methionine in a concentration that ranges from 1 mM to about 100 mM, and in particular, is about 27 mM. In some embodiments, a formulation contains 5 mg/ml of antibody in a buffer of 20 mM sodium citrate, pH 5.5, 140 mM NaCl, and 0.2 mg/ml polysorbate 80. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Another aspect of the present invention provides kits comprising the anti-FRA antibody of the invention or a pharmaceutically composition comprising such an anti-FRA antibody. A kit may include, in addition to the antibody or pharmaceutical composition, diagnostic or therapeutic agents. A kit can also include instructions for use in a diagnostic or therapeutic method, as well as packaging material such as, but not limited to, ice, dry ice, styrofoam, foam, plastic, cellophane, shrink wrap, bubble wrap, cardboard and starch peanuts. In one embodiment, the kit includes the antibody or pharmaceutical composition comprising the antibody and a diagnostic agent that can be used in a method described herein. In still another embodiment, the kit includes the antibody or pharmaceutical composition comprising the antibody and one or more therapeutic agents that can be used in a method described herein.

The invention also relates to compositions and kits for inhibiting cancer in a mammal comprising an amount of an antibody of the invention in combination with an amount of a chemotherapeutic agent, wherein the amounts of the compound, salt, solvate, or prodrug, and of the chemotherapeutic agent are together effective in inhibiting abnormal cell growth. Many chemotherapeutic agents are presently known in the art. In some embodiments, the chemotherapeutic agent is selected from the group consisting of mitotic inhibitors, alkylating agents, anti-metabolites, intercalating antibiotics, chemokine inhibitors, cell cycle inhibitors, enzymes, topoisomerase inhibitors, biological response modifiers, anti-hormones, e.g., anti-androgens, and anti-angiogenesis agents.

### Diagnostic Methods

The anti-FRA antibodies of the invention may be used for in vitro or in vivo detection of FRA or FRA-expressing cells in a biological sample. The anti-FRA antibody may be used in a conventional immunoassay, including, without limitation, an ELISA, an RIA, flow cytometry, immunocytochemistry, tissue immunohistochemistry, Western blot or immunoprecipitation. The anti-FRA antibody of the invention may be used to detect FRA from humans.

In another aspect, the invention provides a method for detecting FRA in a biological sample. The method comprises contacting a biological sample with an anti-FRA antibody of the invention and detecting the bound antibodies. The anti-FRA antibody may be directly labeled with a detectable label or may be unlabeled. If an unlabeled antibody is used, a second antibody or other molecule that can bind the anti-FRA antibody that is labeled is used to detect antibody bound to FRA. As is well known to one of skill in the art, a second antibody is chosen that is able to specifically bind the specific species and class of the first antibody. For example, if the anti-FRA antibody comprises a human IgG, then the secondary antibody may be a labeled anti-human-IgG antibody. Other molecules that can bind to antibodies include, without limitation, Protein A and Protein G, both of which are available commercially, from Pierce Chemical Co.

Suitable labels for the antibody or secondary molecule have been disclosed, and include various enzymes, prosthetic groups, fluorescent materials, luminescent materials, magnetic agents and radioactive materials. Examples of suitable enzymes include horseradish peroxidase, alkaline phosphatase, O-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; an example of a magnetic agent includes gadolinium; and examples of suitable radioactive material include ¹²⁵I, ¹³¹I, ³⁵S or ³H.

The anti-FRA antibodies of the invention may be used to determine the presence and/or level FRA in a tissue or in cells, such as dysplastic cells, derived from the tissue. The tissue may be a diseased tissue such as a tumor or a biopsy thereof. The cells may be, for example, ovarian, pancreatic, prostate or lung cancer cells. The detection may be in a tissue sample or in vivo. An anti-FRA antibody of the invention may be used according to the invention to detect and/or quantify FRA in a tissue, cell surface levels of FRA or localization of FRA by the methods discussed above.

The antibodies of the present invention, especially humanized antibodies also may be used in vivo to detect FRA in tissues and organs, for example in FRA-expressing tumors. For in vivo detection of FRA, a labeled anti-FRA antibody is administered to a patient in need of such a diagnostic test and the patient is subjected to imaging analysis in order to determine the location of the FRA-expressing tissues. Imaging analysis is well known in the medical art, and includes, without limitation, x-ray analysis, magnetic resonance imaging (MRI) or computed tomography (CE). In another embodiment of the method, a tumor or tissue biopsy is obtained from the patient to determine whether it expresses FRA. For imaging, the anti-FRA antibody may be labeled with a detectable agent that can be imaged in a patient. For example, the anti-FRA antibody may be labeled with a contrast agent, such as barium, which can be used for x-ray analysis, or a magnetic contrast agent, such as a gadolinium chelate, which can be used for MRI or CE. Other labeling agents include, without limitation, radioisotopes, such as ⁹⁹Tc. According to the invention, the anti-FRA antibody could also be unlabeled and imaging is by administering a second antibody or other molecule that is detectable and that can bind the anti-FRA antibody.

### Therapeutic Methods

In another aspect, the invention provides methods of using an anti-FRA antibody of the invention for therapy. Methods of the invention include reducing the growth, proliferation or survival of FRA-expressing cells in vitro or in vivo. The invention further provides a method for treating cancer in a subject in need thereof, comprising the step of administering to the subject administering to the subject an anti-FRA monoclonal antibody of the invention. In various embodiments, the cancer is ovarian, breast, renal, colorectal, lung, endometrial, brain, fallopian tube or uterine cancer or leukemia. The invention also provides a method for reducing the growth of dysplastic cells associated with increased expression of FRA in a subject in need thereof, comprising the step of administering to the subject an anti-FRA monoclonal antibody of the invention. In various embodiments, the dysplastic cells are ovarian, breast, renal, colorectal, lung, endometrial, brain, fallopian tube, uterine cancer cells or leukemia cells. In preferred embodiments, the subject is human.

In one embodiment, the invention provides methods for inhibiting FRA activity comprising contacting or exposing a cell expressing FRA with or to an anti-FRA antibody. In some methods, the anti-FRA antibody is administered to a subject in need thereof. The subject may be suffering from a disease or condition characterized by dysplasia or increased expression of FRA. Non-limiting examples include cancer, tumor growth and hyperproliferative disorders. The subject may be a human subject or a veterinary subject, including a non-human animal model of a human disease. The anti-FRA antibody may be used in the manufacture of a medicament for treatment of a condition characterized by dysplasia or increased expression of FRA.

According to the methods of the invention, an anti-FRA antibody of the invention can be administered neat or may be incorporated into a pharmaceutical composition suitable for administration to a subject. The pharmaceutical composition may comprise a pharmaceutically acceptable carrier such as a solvent, dispersion media, coatings, antibacterial and antifungal agents, isotonic and absorption delaying agents, and the like that are physiologically compatible. Examples of pharmaceutically acceptable carriers include but are not limited to one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the antibody or antibody portion.

The anti-FRA antibody may be administered once or multiple times. Where multiple administrations are used, they may be daily, weekly, monthly or if any appropriate periodically including multiple daily doses. The administering may be on a schedule such as three times daily, twice daily, once daily, once every two days, once every three days, once weekly, once every two weeks, once every month, once every two months, once every three months and once every six months. The anti-FRA antibody may also be administered continuously, e.g. via a minipump. The anti-FRA antibody may be administered, for example, via a mucosal, buccal, intranasal, inhalable, intravenous, subcutaneous, intramuscular, parenteral, or intratumor route. The anti-FRA antibody may be administered once, at least twice or for at least the period of time until the condition is treated, palliated or cured. The anti-FRA antibody generally will be administered for as long as the condition is present or longer to prevent recurrence of the condition. The anti-FRA antibody will generally be administered as part of a pharmaceutical composition as described supra. The dosage of anti-FRA antibody will generally be in the range of 0.1 to 100 mg/kg, more preferably 0.5 to 50 mg/kg, more preferably 1 to 20 mg/kg, and even more preferably 1 to 10 mg/kg. The serum concentration of the anti-FRA antibody may be measured by any method known in the art.

In another embodiment, the anti-FRA antibody may be co-administered with another therapeutic agent including another anti-FRA antibody. The additional therapeutic agent also may be an oligonucleotide that reduces expression of FRA by RNA interference, including single stranded or double stranded nucleic acid molecules. In the case of a subject suffering from hyperproliferative disorder, such as cancer or a tumor, the additional therapeutic agent may be an antineoplastic agent. In one aspect, the invention relates to a method for the treatment of a hyperproliferative disorder in a subject in need thereof comprising administering to said subject a therapeutically effective amount of an anti-FRA antibody of the invention in combination with an anti-tumor agent selected from the group consisting of, but not limited to, mitotic inhibitors, alkylating agents, anti-metabolites, intercalating agents, growth factor inhibitors, cell cycle inhibitors, enzymes, topoisomerase inhibitors, biological response modifiers, anti-hormones, kinase inhibitors, matrix metalloprotease inhibitors, genetic therapeutics, anti-androgens, antineoplastic agents and cytotoxic agents. In another preferred embodiment, the anti-FRA antibody or combination therapy is administered along with radiotherapy, chemotherapy, photodynamic therapy, surgery or other immunotherapy.

According to the invention, an anti-FRA antibody of the invention may be administered with an antibody or other agent that is known to inhibit tumor or cancer cell proliferation, e.g., an antibody or agent that inhibits erbB2 receptor, E-selectin, EGF-R, CD20, VEGF (for example, AVASTIN® (bevacizumab), LUCENTIS® (ranibizumab) and MACUGEN® (pegaptanib)), VEGF receptor 1 (VEGFR1), VEGF receptor 2 (VEGFR2) or VEGF receptor 3 (VEGFR3) and the like.

The anti-FRA antibody of the invention may be co-administered with chemotherapeutic agents including, without limitation, GLEEVEC® (imatinib), ERBITUX® (cetuximab), L-asparaginase, IRESSA® (gefitinib), TARCEVA® (erlotinib) and VELCADE® (bortezomib) and the like.

More specifically, the anti-FRA antibody of the invention may be co-administered with alkylating agents. Examples of useful alkylating agents include, without limitation, altretamine (hexamethylmelamine), busulfan, carboplatin, carmustine (BCNU), chlorambucil, cisplatin, CYTOXAN® (cyclophosphamide), dacarbazine (DTIC), ifosfamide, lomustine, mechlorethamine (nitrogen mustard), melphalan, oxalaplatin, streptozocin, TEMODAR® (temozolomide), thiotepa and the like.

The anti-FRA antibody of the invention may be co-administered with antimetabolites. Examples of useful antimetabolites include, without limitation, 5-fluorouracil (5-FU), mercaptopurine (6-MP), XELODA® (capecitabine), ARA-C® (cytarabine), fludarabine, GEMZAR® (gemcitabine), methotrexate, ALIMTA® (pemetrexed) and the like.

The anti-FRA antibody of the invention may be co-administered with topoisomerase I and II inhibitors, including, without limitation, CAMPTOSAR® (irinotecan HCl), SN-38, camptothecin, HYCAMTIN® (topotecan), etoposide, teniposide, ELLENCE® (epirubicin), ADRIAMYCIN® (doxorubicin), idarubicin, mitoxantrone, lamellarin D, HU-331 (Kogan et al, (2007) Molecular Cancer Therapeutics 6: 173-183) and the like.

In some embodiments, the anti-FRA antibody of the invention may be co-administered with anti-tumor antibiotics, such as actinomycin-D, bleomycin, mitomycin-C and the like.

In some embodiments, the anti-FRA antibody of the invention may be co-administered with mitotic inhibitors. Non-limiting examples of useful mitotic inhibitors include EMCYT® (estramustine), IXEMPRA® (ixabepilone), TAXOTERE® (docetaxel), TAXOL® (paclitaxel), VELBAN® (vinblastine), ONCOVIN® (vincristine), NAVELBINE® (vinorelbine) and the like.

In some embodiments, the anti-FRA antibody of the invention may be co-administered with differentiating agents. Non-limiting examples of useful differentiating agents include arsenic trioxide, retinoids, tretinoin and TARGRETIN® (bexarotene) and the like.

In some embodiments, the anti-FRA antibody of the invention may be co-administered with steroid compounds, such as, for example, prednisone, methylprednisolone, dexamethasone and the like.

In some embodiments, the anti-FRA antibody of the invention may be co-administered with hormone-related compounds. Non-limiting examples of useful hormone-related compounds include estrogens, progestins (such as MEGACE® (megestrol acetate)), FASLODEX® (fulvestrant), tamoxifen, toremifene, LUPRON® (leuprolide), ZOLADEX® (goserelin), ARIMIDEX® (anastrozole), FEMARA® (letrozole), AROMASIN® (exemestane), CASODEX® (bicalutamide), EULEXIN® (flutamide), NILANDRON® (nilutamide) and the like.

In some embodiments, the anti-FRA antibody of the invention may be co-administered with a COX-II (cyclooxygenase II) inhibitor. Non-limiting examples of useful COX-II inhibitors include CELEBREX® (celecoxib), valdecoxib, rofecoxib and the like.

In some embodiments, the anti-FRA antibody of the invention may be co-administered with immunotherapeutic agents. Non-limiting examples of useful immunotherapeutic agents include the interferons (such as interferon-alpha), BCG, interleukin-2 (IL-2), thalidomide, lenalidomide, CAMPATH® (alemtuzumab), RITUXAN® (rituximab).

In some embodiments, the anti-FRA antibody of the invention may be co-administered with an MMP inhibitor. For example, the anti-FRA antibody may be co-administered with anti-angiogenic agents, such as MMP-2 (matrix-metalloproteinase 2) inhibitors or MMP-9 (matrix-metalloproteinase 9) inhibitors. Preferred MMP inhibitors are those that do not demonstrate arthralgia. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13). Some specific examples of MMP inhibitors useful in the present invention are AG-3340, RO 32-3555, RS 13-0830, and the compounds recited in the following list: 3-[[4-(4-fluoro-phenoxy)-benzenesulfonyl]-(1-hydroxycarbamoyl-cyclopentyl)-amino]-propionic acid; 3-exo-3-[4-(4-fluoro-phenoxy)-benzenesulfonylamino]-8-oxa-bicyclo[3.2.1]octane-3-carboxylic acid hydroxyamide; (2R,3R) 1-[4-(2-chloro-4-fluoro-benzyloxy)-benzenesulfonyl-]-3-hydroxy-3-methyl-piperidine-2-carboxylic acid hydroxyamide; 4-[4-(4-fluoro-phenoxy)-benzenesulfonylamino]-tetrahydro-pyran-4-carboxylic acid hydroxyamide; 3-[[4-(4-fluoro-phenoxy)-benzenesulfonyl]-(1-hydroxy-carbamoyl-cyclobutyl)-amino]-propionic acid; 4-[4-(4-chloro-phenoxy)-benzenesulfonylamino]-tetrahydro-pyran-4-carboxylic acid hydroxyamide; (R) 3-[4-(4-chloro-phenoxy)-benzenesulfonylamino]-tetrahydro-pyran-3-carboxylic acid hydroxyamide; (2R,3R) 1-[4-(4-fluoro-2-methyl-benzyloxy)-benzenesulfonyl]-3-hydroxy-3-methyl-piperidine-2-carboxylic acid hydroxyamide; 3-[[4-(4-fluoro-phenoxy)-benzenesulfonyl]-(1-hydroxycarbamoyl-1-methyl-ethyl)-amino]-propionic acid; 3-[[4-(4-fluoro-phenoxy)-benzenesulfonyl]-(4-hydroxycarbamoyl-tetrahydro-pyran-4-yl)-amino]-propionic acid; 3-exo-3-[4-(4-chloro-phenoxy)-benzenesulfonylamino]-8-oxa-icyclo[3.2.1]octane-3-carboxylic acid hydroxyamide; 3-endo-3-[4-(4-fluoro-phenoxy)-benzenesulfonylamino]-8-oxa-icyclo[3.2.1]octane-3-carboxylic acid hydroxyamide; and (R) 3-[4-(4-fluoro-phenoxy)-benzenesulfonylamino]-tetrahydro-furan-3-carboxylic acid hydroxyamide; and pharmaceutically acceptable salts and solvates of said compounds.

In some embodiments, the anti-FRA antibody may be co-administered with an integrin inhibitor. Integrin inhibitors, include, without limitation, obtustatin, rhodocetin, Vitaxin (MedImmune), cilengitide (EMD 121974; Merck), S137 (Pfizer), S247 (Pfizer) and JSM6427 (Jerini) (see, e.g., Brown et al. (2008) International Journal of Cancer 123: 2195-2203; Stupp et al. (2007) Journal of Clinical Oncology 25: 1637-1638; Eble et al. (2003) Biochem J. 376: 77-85, all incorporated herein by reference).

Co-administration of an anti-FRA antibody of the invention with an additional therapeutic agent (combination therapy) encompasses administering a pharmaceutical composition comprising the anti-FRA antibody and the additional therapeutic agent as well as administering two or more separate pharmaceutical compositions: one comprising the anti-FRA antibody and the other(s) comprising the additional therapeutic agent(s). Further, co-administration or combination therapy includes the anti-FRA antibody and additional therapeutic agents administered simultaneously or sequentially, or both. For instance, the anti-FRA antibody may be administered once every three days, while the additional therapeutic agent is administered once daily at the same time as the anti-FRA antibody or at a different time. An anti-FRA antibody may be administered prior to or subsequent to treatment with the additional therapeutic agent. Similarly, administration of an anti-FRA antibody of the invention may be part of a treatment regimen that includes other treatment modalities including radiation, surgery, exercise, phototherapy, including laser therapy, and dietary supplements. The combination therapy may be administered to prevent recurrence of the condition. Preferably, the combination therapy is administered multiple times. The combination therapy may be administered from three times daily to once every six months. The administering may be on a schedule such as three times daily, twice daily, once daily, once every two days, once every three days, once weekly, once every two weeks, once every month, once every two months, once every three months and once every six months, or may be administered continuously, e.g. via a minipump. The combination therapy may be administered, for example, via an oral, mucosal, buccal, intranasal, inhalable, intravenous, subcutaneous, intramuscular or parenteral route.

In one embodiment, the anti-FRA antibody is administered in a formulation as a sterile aqueous solution having a pH that ranges from about 5.0 to about 8.0, preferably from about 6.5 to about 7.5, and more preferably from about 7.0 to about 7.2. The formulation may comprise from about 1 mg/ml to about 200 mg/ml, from about 5 mg/ml to about 50 mg/ml, or from about 10 mg/ml to about 25 mg/ml, of antibody. The formulation may comprise from about 1 millimolar to about 100 millimolar of Tween, from about 0.01 mg/ml to about 10 mg/ml of polysorbate 80, from about 100 millimolar to about 400 millimolar of trehalose, and from about 0.01 millimolar to about 1.0 millimolar of disodium EDTA dihydrate. In a preferred embodiment, the antibody is administered in a formulation of 5.0±0.5 mg/mL of antibody in 10 mM sodium phosphate, 150 mM sodium chloride, pH 7.2, 0.01% USP Tween 80.

In a still further embodiment, the anti-FRA antibody is labeled with a radiolabel, an immunotoxin or a toxin, or is a fusion protein comprising a cytotoxic peptide. The anti-ETA antibody or anti-FRA antibody fusion protein directs the radiolabel, immunotoxin, toxin or toxic peptide to the FRA-expressing tumor or cancer cell. In a preferred embodiment, the radiolabel, immunotoxin, toxin or toxic peptide is internalized after the anti-FRA antibody binds to the FRA on the surface of the tumor or cancer cell.

It is understood that the examples and embodiments described herein are for illustrative purposes only and that various modifications or changes in light thereof will be apparent to persons skilled in the art and are to be included within, and can be made without departing from, the true scope of the invention.

### EXAMPLES

### Example 1

### Culturing and Purification of an Anti-FRA Antibody Cultured Under Varied Conditions

CHO-K1 cells producing an anti-FRA antibody comprising the heavy chain amino acid sequence of SEQ ID NO: 5 and the light chain amino acid sequence of SEQ ID NO:7 were recovered from cryopreservation and subcultured under the conditions shown in Table 1. The inoculum expansion and subculture were performed in 125 mL and 500 mL shake flasks.

TABLE 1
Cryopreserved cell stocks recovery and subculture conditions.

| Parameter | Value/Range |
|---|---|
| Medium | CD-CHO (Invitrogen) |
| Incubation Temperature (° C.) | 37° C. ± 0.5 |
| Shaking Speed (rpm) | 120 ± 5 |
| Head space equilibration gas | 5% CO₂ in 95% air |
| Revival inoculation viable cell concentration | 0.30 |
| (10⁶/mL) | |
| Number of days between revival and first | 3 |
| subculture | |
| Serial subculture inoculation viable cell | 0.2 |
| concentration (10⁶/mL) | |
| Number of days between first and subsequent | 4 |
| subcultures | |

After recovery and subculture, the cells were cultured in 2 L stirred-tank production bioreactors (B-DCU, Sartorius) fed-batch culture with various experimental cell culture condition changes and with the positive controls (Tables 2 and 3).

TABLE 2
Conditions for operating 2 L stirred-tank production bioreactors fed-batch
cultures.

| Parameter | Value/Range |
|---|---|
| Medium | CD-CHO (Invitrogen) |
| Production bioreactor | 0.18-0.25 |
| inoculation cell concentration | |
| (10⁶ viable cells/mL) | |
| Temperature set point (° C.) | 36.5 |
| pH | 7.1-6.9 dead band |
| Dissolved oxygen tension (%) | 30.0 |
| Agitation (rpm) | 180 |
| 300 g/L glucose feed | Continuous variable rate application as |
| | required to target a glucose concentration |
| | of 1.00 to 3.00 g/L |
| Condition changes | Various condition changes applied at |
| | different time points (see Table 3) |

TABLE 3
Table of bioreactor condition changes.

| | Parameter | Value/Range |
|---|---|---|
| 1 | Positive Control | T = 36.5° C., pH = 7.0, DO = 30%, |
| | | glucose concentration of 1.00 to 3.00 g/L |
| 2 | Galactose | 2 g/L Bolus addition 5 days after initiation of |
| | supplement | culture |
| 3 | Low temperature | Shift from 36.5° C. to 30° C. |
| | | 5 days after initiation of culture |
| 4 | High osmolarity | Add NaCl to bring up to 600 mOsm/L 7 days |
| | | after initiation of culture |
| 5 | 0.5 mM sodium | Bolus addition 6 days after initiation of culture |
| | butyrate | |
| 6 | Low dissolved | DO was changed from 30% to 5% 6 days after |
| | oxygen (DO) | initiation of culture |
| 7 | High CO₂ | Bring CO₂ accumulation in medium up to 20% |
| | | 5 days after initiation of culture |
| 8 | 10 mM sodium | Bolus addition 6 days after initiation of culture |
| | butyrate | |
| 9 | Copper chloride | 0.5 mM Bolus addition 6 days after initiation of |
| | | culture |
| 10 | Time points | Harvest samples on day 10, 14, 17, and 20 |

The MORAb-003 anti-FRA antibody cultures were purified as follows. Conditioned culture media (1.5 L for each condition) from cells secreting MORAb-003 were clarified by centrifugation (10,000 g, 20 min). Resulting supernatants were filtered through a 0.22 min membrane. MORAb-003 antibodies in the conditioned media were purified by protein A affinity chromatography, with the aid of an AktaExplorer 100A (GE Healthcare). Briefly, a 20 mL bed volume column packed with ProSep vA (Cat#113115830, Millipore) was equilibrated with PBS (0.02 M potassium phosphate, 0.15 M sodium chloride, 7.2). After clarification, MORAb-003-containing conditioned media were loaded onto the column at a flow rate of 5.0 mL/min. The resin was washed with PBS for 12 column volumes (CV), followed by elution of bound MORAb-003 antibodies, using 3.5 CV of elution buffer (10 mM acetic acid, 100 mM glycine, 3.8). The resin was cleaned with 2 CV 6 M guanidine hydrochloride. The flow rates in all the steps except loading were 7.0 mL/min. The pooled elution fractions were dialyzed against 2 L PBS for approximately 15 hours at 4° C., using 10 kDa cut-off SnakeSkin Pleated Dialysis Tubing (Prod#68100, Thermo Scientific).

### Example 2

### Glycan Profiling of an Anti-FRA Antibody Cultured Under Varied Conditions

After the MORAb-003 anti-FRA antibodies were cultured and purified as described in Example 1, the neutral, N-linked complex carbohydrate structures found on the antibodies were removed, separated, identified and quantified as follows. The lot numbers assigned to samples of anti-FRA antibodies cultured under varied conditions are shown in Table 4. N-linked glycans in the MORAb-003 antibody heavy chain were enzymatically removed using peptidyl-N-glycosidase F (PNGase F), and purified by gel filtration chromatography. The resulting glycan mixture was fluorescently labeled using 2-aminobenzamide (2-AB), and resolved by normal phase HPLC, using a Tosoh TSK-Gel 80-Amide column. Fluorescently labeled glycans were quantified by fluorescence (Ex 330 nm/Em 420 nm). Identification of glycans from peaks arising during separation was accomplished by in-line mass spectrometric detection, using an Agilent ESI-TOF mass spectrometer in either total ion chromatogram or extracted ion chromatogram mode. Diagrams of the recovered glycan structures are shown in FIG. 1.

TABLE 4
Lot numbers corresponding to anti-FRA antibody culture conditions.

| Client | Bio- | | |
|---|---|---|---|
| Lot # | reactor | Round | Parameter |
| NB810-10 | BR5 | 1 | positive control; harvested 14 days after |
| | | | initiation of culture |
| NB810-11 | BR6 | 1 | galactose supplement; harvested 14 days |
| | | | after initiation of culture |
| NB810-12 | BR7 | 1 | low temperature; harvested 14 days after |
| | | | initiation of culture |
| NB810-13 | BR8 | 1 | high osmolarity; harvested 14 days after |
| | | | initiation of culture |
| NB809-65 | BR1 | 2 | positive control; harvested 13 days after |
| | | | initiation of culture |
| NB809-66 | BR3 | 2 | 0.5 mM Na butyrate; harvested 13 days |
| | | | after initiation of culture |
| NB809-67 | BR4 | 2 | low DO; harvested 13 days after initiation |
| | | | of culture |
| NB809-68 | BR5 | 2 | high CO₂; harvested 13 days after |
| | | | initiation of culture |
| NB809-69 | BR7 | 2 | 10 mM Na butyrate; harvested 13 days |
| | | | after initiation of culture |
| NB809-70 | BR8 | 2 | CuCl supplement; harvested 13 days after |
| | | | initiation of culture |
| NB859-24 | BR1 | 3 | Harvested 10 days after initiation of |
| | | | culture |
| NB859-25 | BR1 | 3 | Harvested 14 days after initiation of |
| | | | culture |
| NB859-26 | BR1 | 3 | Harvested 17 days after initiation of |
| | | | culture |
| NB859-27 | BR1 | 3 | Harvested 20 days after initiation of |
| | | | culture |
| NB859-28 | BR2 | 3 | Low DO; harvested 14 days after initiation |
| | | | of culture |

Lot NB859-25 was derived from a culture grown under the conditions to those employed for positive control lots NB810-10 and NB809-65. Lot NB859-25 was designated as a positive control for purposes of comparison of experimental results to the other lots produced in round 3.

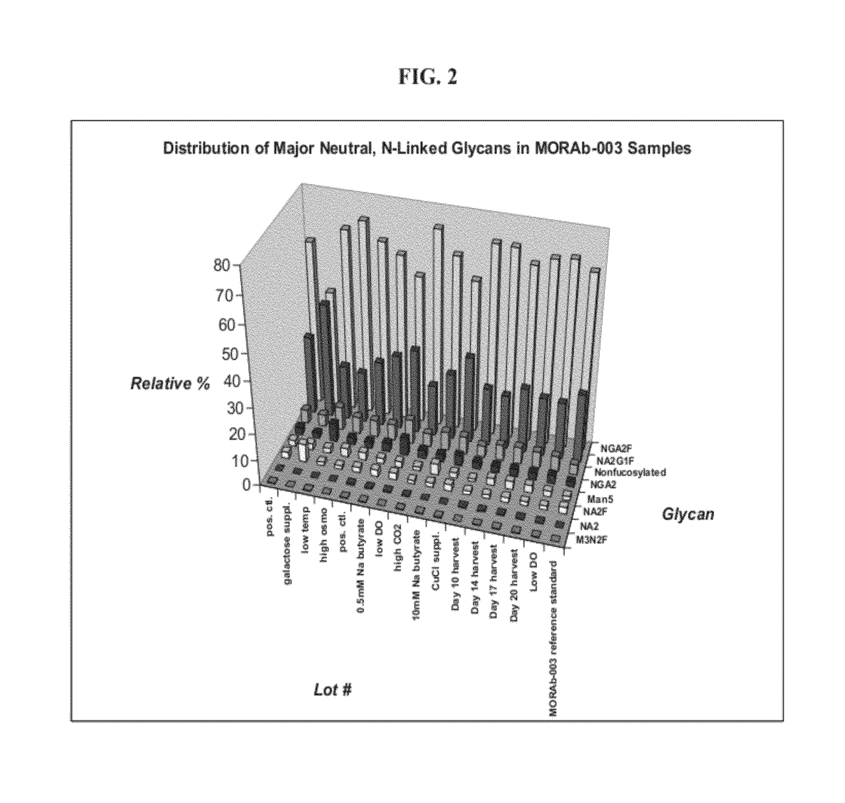

Preliminary results of the distribution of major neutral N-linked glycans in MORAb-003 anti-FRA antibody samples are shown in FIG. 2. Additional results of the distribution of neutral glycans in MORAb-003 anti-FRA antibody samples are shown in FIG. 3, “MORAb-003 reference standard” is a MORAb-003 anti-FRA antibody produced under “reference culture conditions” as defined herein and supplied by an outside manufacturer.

### Example 3

### Correlation of Binding Affinity of an Anti-FRA Antibody with Antibody Culture Conditions and Glycan Structure

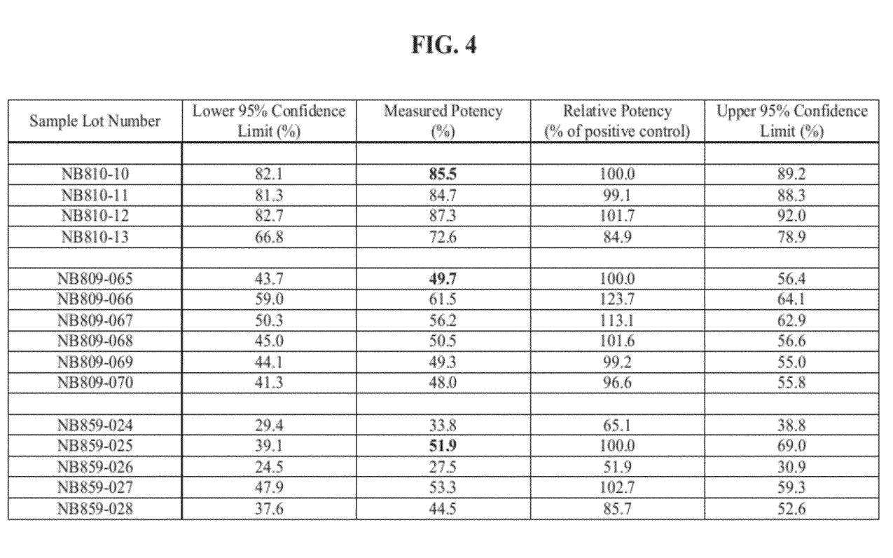

The relative binding affinity of MORAb-003 anti-FRA antibody samples was determined by surface plasmon resonance spectroscopy. Recombinant human folate receptor alpha (FRA) (SEQ ID NO: 3) was immobilized on the surface of a research grade CM5 chip via amine coupling. Dilutions of MORAb-003 anti-FRA antibody reference standard produced under “reference culture conditions” as defined herein and supplied by an outside manufacturer or variant sample preparations spanning 0.02-44 mg/mL were serially injected over the surface, and binding levels were measured after allowing association to proceed for 40 s. The surface was regenerated between cycles by flushing with 10 mM glycine pH 2.0. Binding level as a function of MORAb-003 concentration was plotted for the reference standard and all samples, using BiaEvaluation 4.1 (GE Healthcare). Data were fitted to a five parameter logistical curve fit, and the relative binding potency of each sample compared to the reference standard was determined by parallel line analysis using STATLIA version 3.2 (Brendan Technologies). Results are shown in FIG. 4. To calculate the results in the “Measured Potency” column, the binding potency of the reference standard was set as 100%. All culture conditions, including positive controls, had a lower binding affinity compared to the MORAb-003 anti-FRA antibody produced under “reference culture conditions” as defined herein and supplied by an outside manufacturer.

### Example 4

### Correlation of ADCC of an Anti-FRA Antibody with Antibody Culture Conditions and Glycan Structure

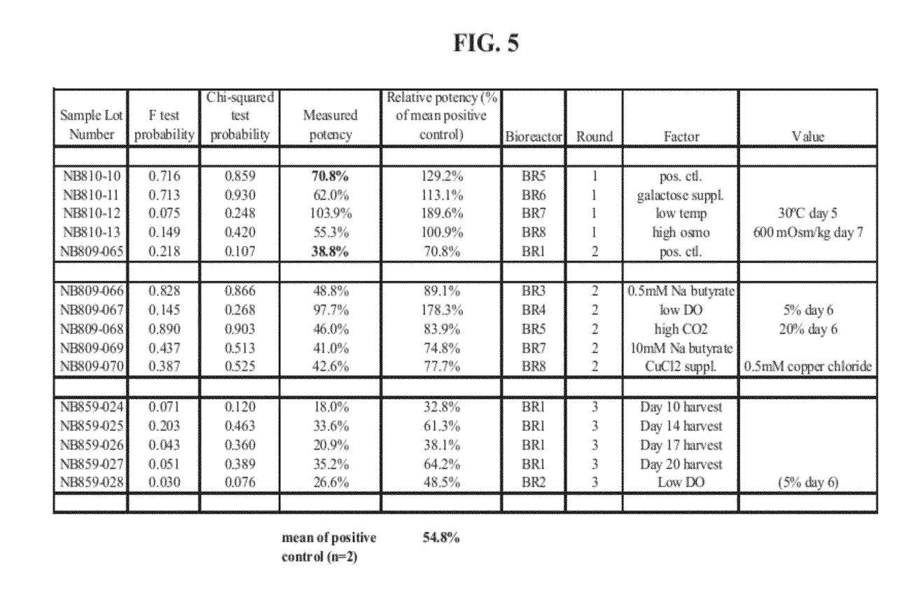

The in vitro antibody-dependent cellular cytotoxicity (ADCC) mediated by MORAb-003 anti-FRA antibody samples was measured as follows. FRA-expressing IGROV-1 human ovarian adenocarcinoma cells (Bernard, et al, (1985) Cancer Res 4: 4970-4979) were labeled with carboxyfluorescein diacetate succinimidyl ester (CFDA SE). Labeled cells were mixed with dilutions of MORAb-003 anti-FRA antibody samples and unlabeled effector cells derived from human peripheral blood mononuclear cells (PBMCs). After a 4-hour incubation, the cell populations were scored by flow cytometry for remaining viable, labeled IGROV-1 cells. The fraction of remaining cells after treatment with an variant sample lot of MORAb-003 anti-FRA antibody were compared to that of cells treated with identical concentrations of a MORAb-003 anti-FRA antibody reference standard preparation produced under “reference culture conditions” as defined herein and supplied by an outside manufacturer. Results are shown in FIG. 5. To calculate the results in the “Measured Potency” column, the ADCC of the reference standard was set as 100%. To calculate the results in the “Relative Potency” column, the mean of the measured ADCC of the two positive controls was set at 100%.

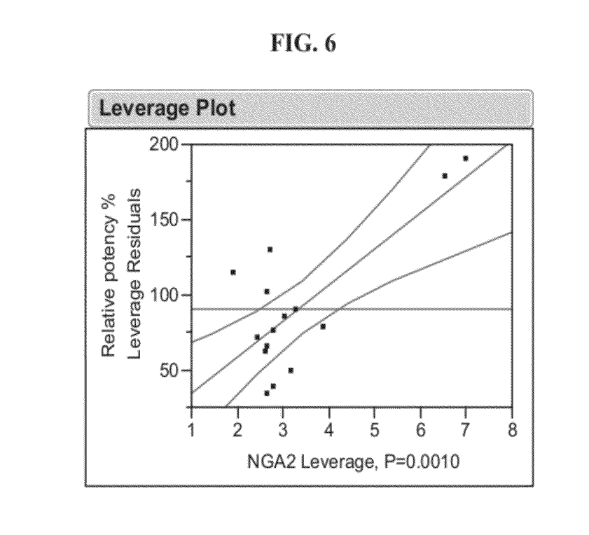

Low temperature and low DO cell culture conditions produced MORAb-003 anti-FRA antibody isoforms with high percentages of NGA2 (G0) glycans. The presence of NGA2 (G0) glycans was correlated with increased ADCC (FIG. 6). This correlation was statistically significant.

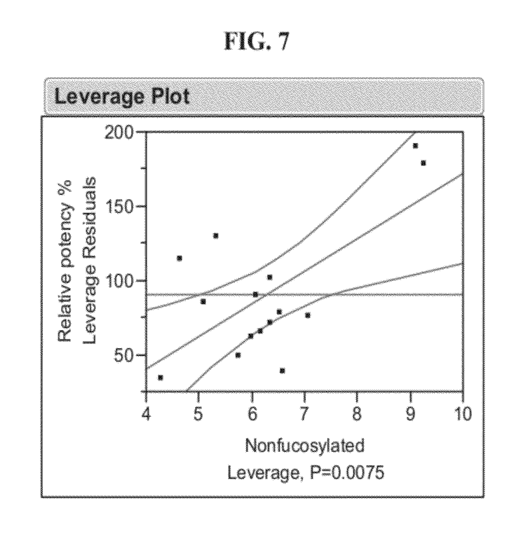

The presence of non-fucosylated glycans was correlated with increased ADCC (FIG. 7). This correlation was statistically significant.

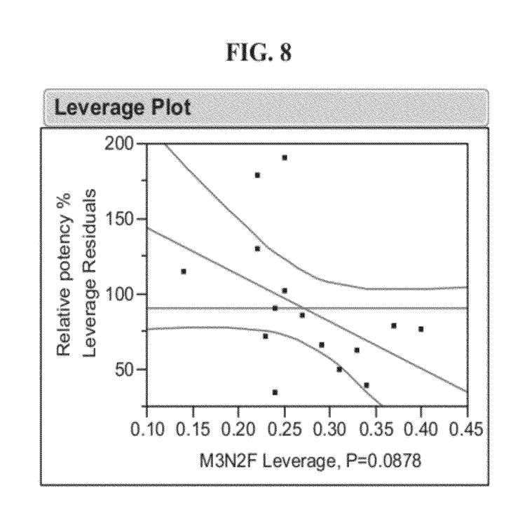

The presence of M3N2F glycans was inversely correlated with increased ADCC (FIG. 8).

### Example 5

### Correlation of Internalization Activity of an Anti-FRA Antibody with Antibody Culture Conditions and Glycan Structure

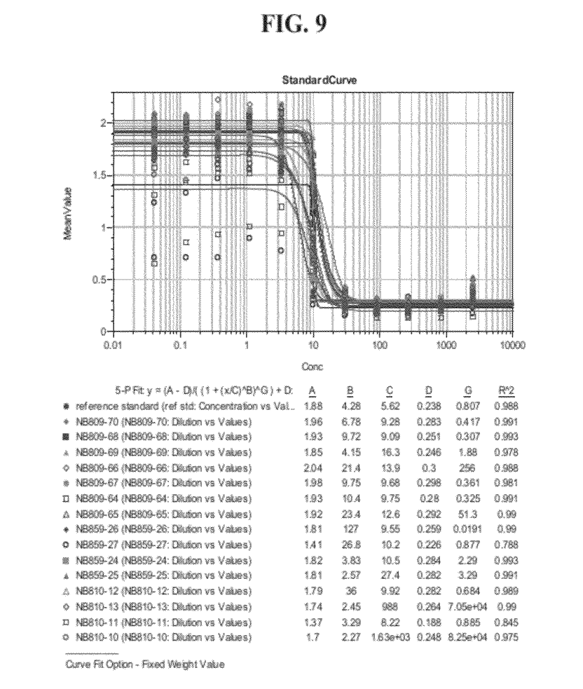

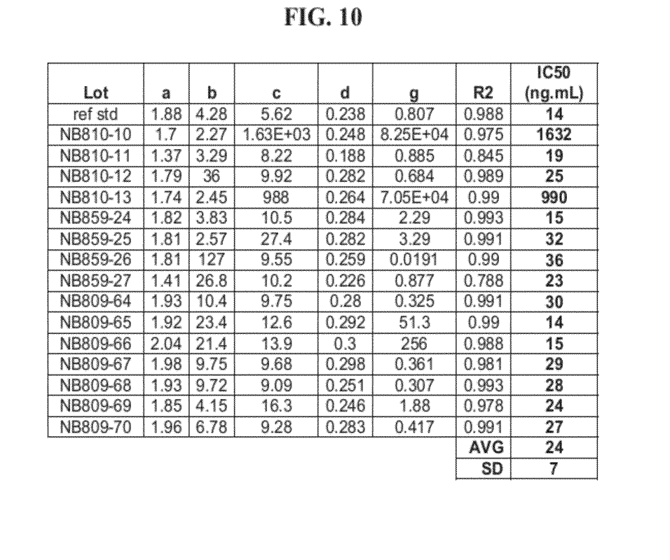

Internalization activity of MORAb-003 anti-FRA antibody samples was scored by the degree of killing of FRA-expressing cells, using an anti-human secondary immunotoxin. Dilutions of MORAb-003 anti-FRA antibody samples and a fixed amount of a goat anti-human IgG secondary antibody conjugated to the cytotoxic plant protein saporin (Hum-ZAP, Advanced Targeting Systems, Inc.) were added to the wells of 96-well tissue-culture treated microtiter plates containing 2,000 cells/well of human FRA-expressing IGROV-1 cells. Internalization of the MORAb-003-Hum-ZAP complex upon antigen binding results in release of the cytotoxin into the IGROV-1 cells in a MORAb-003-dependent manner. The fraction of MORAb-003 anti-FRA antibody internalized can thus be scored based on the degree of killing of IGROV-1 cells. IGROV-1 cell proliferation was measured by Sulforhodamine Blue staining of remaining, live cells using a SpectraMax 190 microplate reader (Molecular Devices Corp.). Data (OD540 versus concentration of MORAb-003 anti-FRA antibody) were fitted to a 5-parameter logistical curve fitting algorithm. The concentration of MORAb-003 anti-FRA antibody resulting in 50% killing of IGROV-1 cells (IC50) was calculated from the curve fit parameters measured for each curve (see FIG. 9 and FIG. 10).

Internalization activity of MORAb-003 anti-FRA antibody samples was measured also by FACS. Human FRA-expressing IGROV-1 cells were incubated for 30 min at 4° C. with 1 μg/mL MORAb-003 anti-FRA antibody samples and washed with PBS. The cells were then incubated for 30 min at 4° C. with 40 μg/mL FITC-conjugated anti-human IgG antibody and washed with PBS. The cells were then incubated for a given period of time at 37° C. to start internalization. The cells were washed with acidic glycine buffer at 4° C. to remove all membrane-bound antibodies. After the acidic glycine buffer wash, flow cytometry was performed on the cells at 4° C. In all experiments, internalization was defined as the time-dependent increase in mean florescence intensity (MFI), as only antibody that was internalized within the plasma membrane would be retained and would produce a fluorescent signal following the acidic wash.

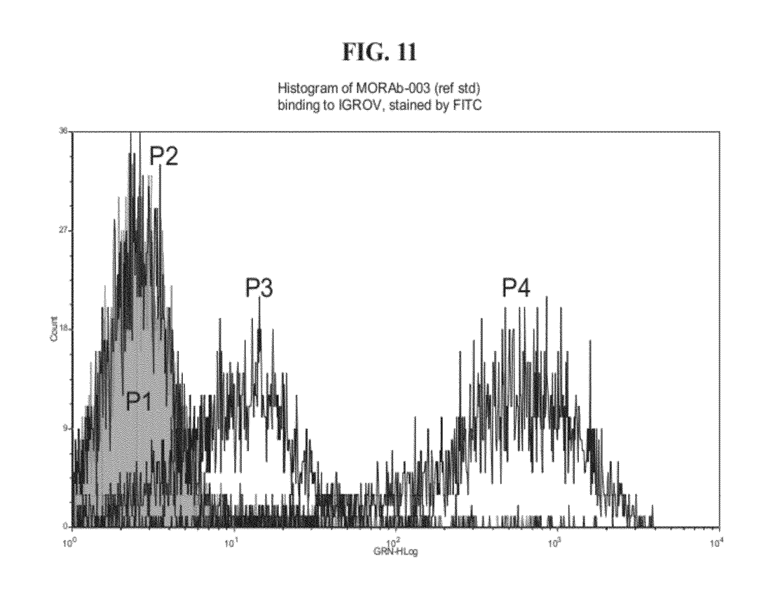

FIG. 11 shows a histogram of the results of the FACS binding experiment performed with the MORAb-003 anti-FRA antibody reference standard produced under “reference culture conditions” as defined herein and supplied by an outside manufacturer. The shaded area (P1 population) corresponds to cells incubated with MC-conjugated anti-human IgG antibody. The P2 population (0% control) corresponds to cells incubated with irrelevant human IgG as control and FITC-conjugated anti-human IgG antibody. The P3 population corresponds to cells incubated with the anti-FRA antibody and FITC-conjugated anti-human IgG antibody and washed with acidic glycine buffer. The P4 population (100% control) corresponds to cells incubated with the anti-FRA antibody and FITC-conjugated anti-human IgG antibody with PBS buffer wash.

The percentage of MORAb-003 anti-FRA antibody internalization was calculated from the relative fluorescence intensity as follows:

((MFI[sample]−MFI[0% control])/(MFI[100% control]−MFI[0% control]))×100

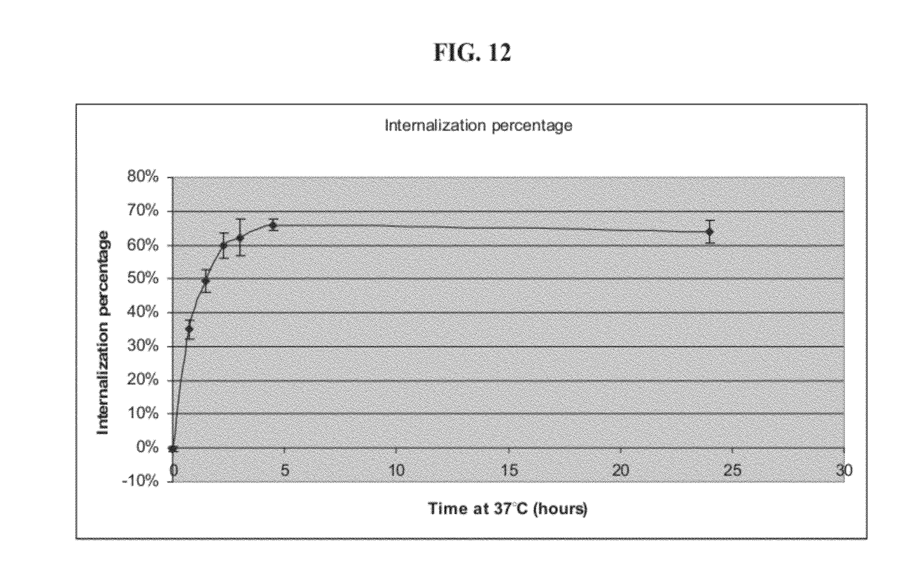

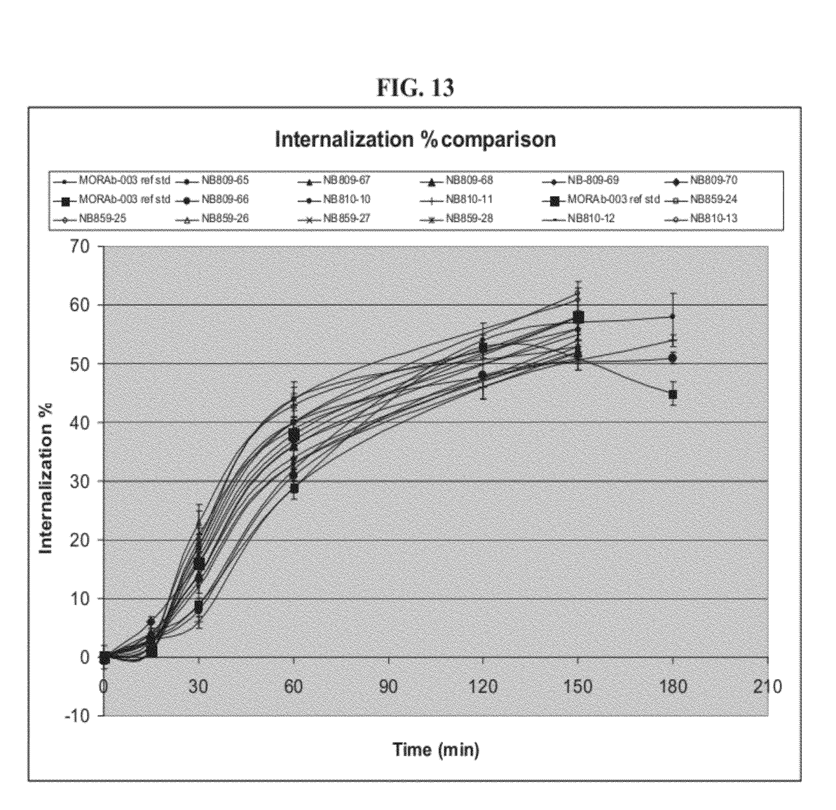

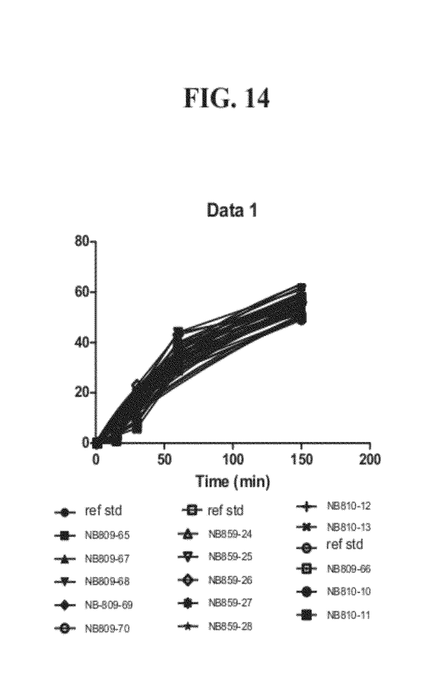

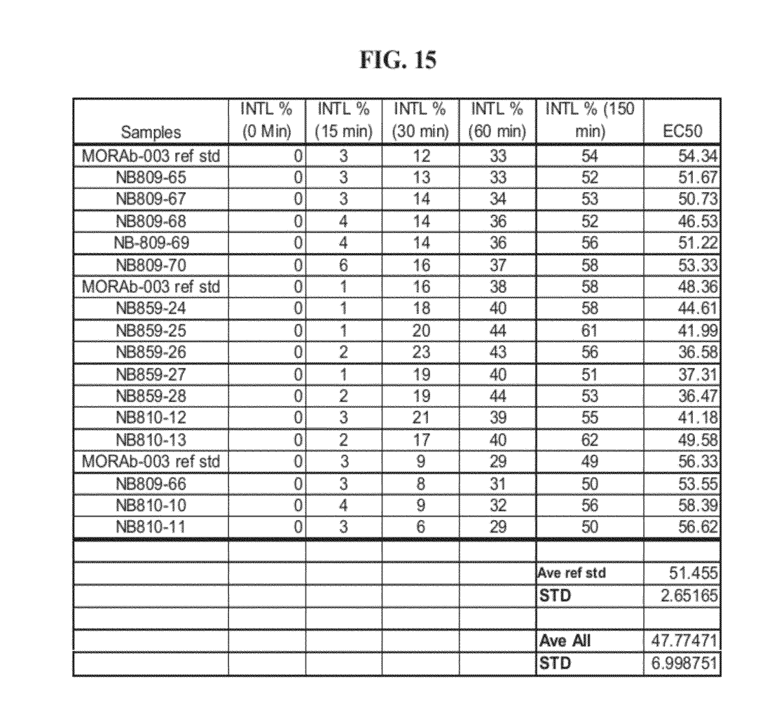

FIG. 12 depicts the relationship between time and internalization by IGROV-1 cells of the MORAb-003 anti-FRA antibody reference standard produced under “reference culture conditions” as defined herein and supplied by an outside manufacturer. The y-axis represents the percentage of MFI measured by flow cytometry of a population of IGROV-1 cells over time (x-axis) relative to total binding at each time point. FIG. 13 depicts the relationship between time and internalization by IGROV-1 cells of the MORAb-003 anti-FRA antibody samples described in Table 4, as well as MORAb-003 anti-FRA antibody reference standard produced under “reference culture conditions” as defined herein and supplied by an outside manufacturer, with the three samples labeled “MORAb-003 ref std” representing the same batch of antibody used in different runs of the FACS experiment. FIG. 14 depicts fitting, of the data in FIG. 13 by a nonlinear regression to a four parameter logistical curve. Resulting EC50 curve fit parameters were used to populate FIG. 15.

FIG. 15 summarizes the results of the FACS MORAb-003 anti-FRA antibody sample internalization studies and provides EC50 values. Lower EC50 values indicate faster internalization. MORAb-003 anti-ETA antibody reference standard produced under “reference culture conditions” as defined herein and supplied by an outside manufacturer reached an internalization peak at about 2 hours (120 min). Samples NB859-26 and NB859-27 (17 and 20 days culture product) have a lower EC50 value, indicating a faster internalization process than the positive control. Sample NB859-28 (culture treated with low dissolved oxygen) also demonstrated a faster internalization process than the positive control.

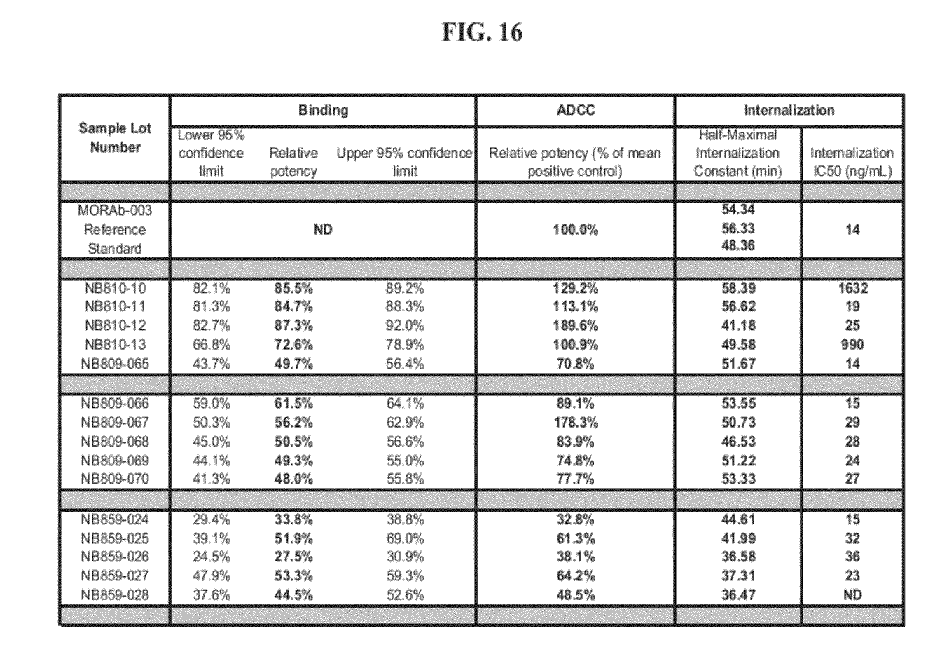

FIG. 16 summarizes the activity data described in Examples 3, 4 and 5.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclature used in connection with, and techniques of cell and tissue culture, molecular biology, immunology, microbiology, genetics and protein and nucleic acid chemistry and hybridization described herein are those well known and commonly used in the art.

The methods and techniques of the present invention are generally performed according to conventional methods well known in the art and as described in various general and more specific references that are cited and discussed throughout the present specification unless otherwise indicated. See, e.g., Sambrook J. & Russell D., Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John &. Sons, Inc. (2003), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well known and commonly used in the art.

All publications, patents, patent applications or other documents cited herein are hereby incorporated by reference in their entirety for all purposes to the same extent as if each individual publication, patent, patent application, or other document was individually indicated to be incorporated by reference for all purposes.

Throughout this specification and embodiments, the word “comprise,” or variations such as “comprises” or “comprising,” will be understood to imply the inclusion of a stated integer or group of integers but not the exclusion of any other integer or group of integers.

TABLE 5
Sequence Table
ANTI-FRA ANTIBODY HEAVY CHAIN AMINO ACID SEQUENCE
MGWSCIILFLVATATGVHSEVQLVESGGGVVQPGRSLRLSCSASGFTFSGYGLSWVRQAP
GKGLEWVAMISSGGSYTYYADSVKGRFAISRDNAKNTLFLQMDSLRPEDTGVYFCARHGD
DPAWFAYWGQGTPVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK (SEQ ID NO: 1)
EVQLVESGGGVVQPGRSLRLSCSASGFTFSGYGLSWVRQAPGKGLEWVAMISSGGSYTYYADSV
KGRFAISRDNAKNTLFLQMDSLRPEDTGVYFCARHGDDPAWFAYWGQGTPVTVSSASTKGPSVF
PLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNKYTQKSLSLSPG
K (SEQ ID NO: 5)
ANTI-FRA ANTIBODY LIGHT CHAIN AMINO ACID SEQUENCE
MGWSCIILFLVATATGVHSDIQLTQSPSSLSASVGDRVTITCSVSSSISSDNLHWYQQKP
GKAPKPWIYGTSNLASGVPSRFSGSGSGTDYTFTISSLQPEDIATYYCQQWSSYPYMYTF
GQGTKVEIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
(SEQ ID NO: 2)
MGWSCIILFLVATATGVHSDIQLTQSPSSLSASVGDRVTITCSVSSSISSNNLHWYQQKPGKAP
KPWIYGTSNLASGVPSRFSGSGSGTDYTFTISSLQPEDIATYYCQQWSSYPYMYTFGQGTKVEI
KRTVAAPSVFIFPPSDEQLKSGTASVVCLLNNFYPREAKVQWKVDNALQSGNSQESVTEQDSKD
STYSLSSTLTLSKADYEKHKVYACEVTHQGLSSPVTKSFNRGEC (SEQ ID NO: 6)
DIQLTQSPSSLSASVGDRVTITCSVSSSISSNNLHWYQQKPGKAPKPWIYGTSNLASGVPSRFS
GSGSGTDYTFTISSLQPEDIATYYCQQWSSYPYMYTFGQGTKVEIKRTVAAPSVFIFPPSDEQL
KSGTASVVCLLNNFYPREAKVQWKVDNALQSGNSQESVTEQDSKDSTYSLSSTLTLSKADYEKH
KVYACEVTHQGLSSPVTKSFNRGEC (SEQ ID NO: 7)
ANTI-FRA ANTIBODY HEAVY CHAIN NUCLEOTIDE SEQUENCE
atgggatggagctgtatcatcctcttcttggtagcaacagctacaggtgtccactccgaggtcc
aactggtggagagcggtggaggtgttgtgcaacctggccggtccctgcgcctgtcctgctccgc
atctggcttcaccttcagcggctatgggttgtcttgggtgagacaggcacctggaaaaggtctt
gagtgggttgcaatgattagtagtggtggtagttatacctactatgcagacagtgtgaagggta
gatttgcaatatcgcgagacaacgccaagaacacattgttcctgcaaatggacagcctgagacc
cgaagacaccggggtctatttttgtgcaagacatggggacgatcccgcctggttcgcttattgg
ggccaagggaccccggtcaccgtctcctcagcctccaccaagggcccatcggtcttccccctgg
caccctcctccaagagcacctctgggggcacagcggccctgggctgcctggtcaaggactactt
ccccgaaccggtgacggtgtcgtggaactcaggcgccctgaccagcggcgtgcacaccttcccg
gctgtcctacagtcctcaggactctactccctcagcagcgtgatgaccgtgccctccagcagct
tgggcacccagacctacatctgcaacgtgaatcacaagcccagcaacaccaaggtggacaagaa
agttgagcccaaatcttgtgacaaaactcacacatgcccaccgtgcccagcacctgaactcctg
gggggaccgtcagtcttcctcttccccccaaaacccaaggacaccctcatgatctcccggaccc
ctgaggtcacatgcgtggtggtggacgtgagccacgaagaccctgaggtcaagttcaactggta
cgtggacggcgtggaggtgcataatgccaagacaaagccgcgggaggagcagtacaacagcacg
taccgtgtggtcagcgtcctcaccgtcctgcaccaggactggctgaatggcaaggagtacaagt
gcaaggtctccaacaaagccctcccagcccccatcgagaaaaccatctccaaagccaaagggca
gccccgagaaccacaggtgtacaccctgcccccatcccgggatgagctgaccaagaaccaggtc
agcctgacctgcctggtcaaaggcttctatcccagcgacatcgccgtggagtgggagagcaatg
ggcagccggagaacaactacaagaccacgcctcccgtgctggactccgacggctccttcttctt
atattcaaagctcaccgtggacaagagcaggtggcagcaggggaacgtcttctcatgctccgtg
atgcatgaggctctgcacaaccactacacgcagaagagcctctccctgtctcccgggaaatga
(SEQ ID NO: 8)
ANTI-FRA ANTIBODY LIGHT CHAIN DNA SEQUENCE
atgggatggagctgtatcatcctcttcttggtagcaacagctacaggtgtccactccgacatcc
agctgacccagagcccaagcagcctgagcgccagcgtgggtgacagagtgaccatcacctgtag
tgtcagctcaagtataagttccaacaacttgcactggtaccagcagaagccaggtaaggctcca
aagccatggatctacggcacatccaacctggcttctggtgtgccaagcagattcagcggtagcg
gtagcggtaccgactacaccttcaccatcagcagcctccagccagaggacatcgccacctacta
ctgccaacagtggagtagttacccgtacatgtacacgttcggccaagggaccaaggtggaaatc
aaacgaactgtggctgcaccatctgtcttcatcttcccgccatctgatgagcagttgaaatctg
gaactgcctctgttgtgtgcctgctgaataacttctatcccagagaggccaaagtacagtggaa
ggtggataacgccctccaatcgggtaactcccaggagagtgtcacagagcaggacagcaaggac
agcacctacagcctcagcagcaccctgacgctgagcaaagcagactacgagaaacacaaagtct
acgcctgcgaagtcacccatcagggcctgagctcgcccgtcacaaagagcttcaacaggggaga
gtgttaa (SEQ ID NO: 9)
HUMAN FRA AMINO ACID SEQUENCE
MAQRMTTQLLLLLVWVAVVGEAQTRIAWARTELLNVCMNAKHHKEKPGPEDKLHEQCRPWRKNA
CCSTNTSQEAHKDVSYLYRFNWNHCGEMAPACKRHFIQDTCLYECSPNLGPWIQQVDQSWRKER
VLNVPLCKEDCEQWWEDCRTSYTCKSNWHKGWNWTSGFNKCAVGAACQPFHFYFPTPTVLCNEI
WTHSYKVSNYSRGSGRCIQMWFDPAQGNPNEEVARFYAAAMSGAGPWAAWPFLLSLALMLLWLL
S (SEQ ID NO: 3)
HUMAN FRA NUCLEOTIDE SEQUENCE
atggctcagcggatgacaacacagctgctgctccttctagtgtgggtggctgtagtaggggagg
ctcagacaaggattgcatgggccaggactgagcttctcaatgtctgcatgaacgccaagcacca
caaggaaaagccaggccccgaggacaagttgcatgagcagtgtcgaccctggaggaagaatgcc
tgctgttctaccaacaccagccaggaagcccataaggatgtttcctacctatatagattcaact
ggaaccactgtggagagatggcacctgcctgcaaacggcatttcatccaggacacctgcctcta
cgagtgctcccccaacttggggccctggatccagcaggtggatcagagctggcgcaaagagcgg
gtactgaacgtgcccctgtgcaaagaggactgtgagcaatggtgggaagattgtcgcacctcct
acacctgcaagagcaactggcacaagggctggaactggacttcagggtttaacaagtgcgcagt
gggagctgcctgccaacctttccatttctacttccccacacccactgttctgtgcaatgaaatc
tggactcactcctacaaggtcagcaactacagccgagggagtggccgctgcatccagatgtggt
tcgacccagcccagggcaaccccaatgaggaggtggcgaggttctatgctgcagccatgagtgg
ggctgggccctgggcagcctggcctttcctgcttagcctggccctaatgctgctgtggctgctc
agctag (SEQ ID NO: 4)
Leader sequences are in italics. CDRs are underlined.

1. A monoclonal antibody that specifically binds folate receptor alpha (FRA), wherein the monoclonal antibody comprises the heavy chain amino acid sequence in SEQ ID NO: 1 or SEQ ID NO: 5, with or without the c-terminal lysine, and further comprises a light chain amino acid sequence selected from: an amino acid sequence that is 99% identical to the amino acid sequence in SEQ ID NO: 2; the amino acid sequence of SEQ ID NO: 6; or the amino acid sequence of SEQ ID NO: 7, and wherein the monoclonal antibody has altered antibody-dependent cellular cytotoxicity (ADCC) compared to the ADCC of the monoclonal antibody when prepared under reference culture conditions.

2. The monoclonal antibody of claim 1, wherein the ADCC is increased.

3. The monoclonal antibody of claim 1, wherein the ADCC is decreased.

4. The monoclonal antibody of claim 2, which has an increased internalization rate compared to the internalization rate of the monoclonal antibody prepared under reference culture conditions.

5. A monoclonal antibody that specifically binds folate receptor alpha (FRA), wherein the monoclonal antibody comprises the heavy chain amino acid sequence in SEQ ID NO: 1 or SEQ ID NO: 5, with or without the c-terminal lysine, and further comprises a light chain amino acid sequence selected from: an amino acid sequence that is 99% identical to the amino acid sequence in SEQ ID NO: 2; the amino acid sequence of SEQ ID NO: 6; or the amino acid sequence of SEQ ID NO: 7, and wherein the monoclonal antibody has an altered internalization rate compared to the internalization rate of the monoclonal antibody when prepared under reference culture conditions.

6. The monoclonal antibody of claim 5, wherein the internalization rate is increased.

7. The monoclonal antibody of claim 5, wherein the internalization rate is decreased.

8. A monoclonal antibody that specifically binds folate receptor alpha (FRA), wherein the monoclonal antibody comprises the heavy chain amino acid sequence in SEQ ID NO: 1 or SEQ ID NO: 5, with or without the c-terminal lysine, and further comprises a light chain amino acid sequence selected from: an amino acid sequence that is 99% identical to the amino acid sequence in SEQ ID NO: 2; the amino acid sequence of SEQ ID NO: 6; or the amino acid sequence of SEQ ID NO: 7, and wherein the monoclonal antibody has an altered N-linked neutral glycan profile compared to the N-linked neutral glycan profile of the monoclonal antibody when prepared under reference culture conditions.

9. The monoclonal antibody of claim 8, wherein the altered N-linked neutral glycan profile comprises one or more of:
a) increased or decreased M3N2;
b) increased or decreased M3N2F;
c) increased NA2;
d) increased or decreased NA2F;
e) increased or decreased MAN5;
f) increased NGA2;
g) increased or decreased NGA2F; or
h) increased or decreased NA2G1F.

10. A monoclonal antibody that specifically binds folate receptor alpha (FRA), wherein the monoclonal antibody comprises the heavy chain amino acid sequence in SEQ ID NO: 1 or SEQ ID NO: 5, with or without the c-terminal lysine, and further comprises a light chain amino acid sequence selected from: an amino acid sequence that is 99% identical to the amino acid sequence in SEQ ID NO: 2; the amino acid sequence of SEQ ID NO: 6; or the amino acid sequence of SEQ ID NO: 7, and wherein the monoclonal antibody has increased ADCC and has an N-linked neutral glycan profile comprising increased NGA2 and increased total non-fucosylated glycoforms compared to the monoclonal antibody when prepared under reference culture conditions.

11. The monoclonal antibody of claim 10, wherein the antibody further has an increased internalization rate.

12. A monoclonal antibody that specifically binds folate receptor alpha (FRA), wherein the monoclonal antibody comprises the heavy chain amino acid sequence in SEQ ID NO: 1 or SEQ ID NO: 5, with or without the c-terminal lysine, and further comprises a light chain amino acid sequence selected from: an amino acid sequence that is 99% identical to the amino acid sequence in SEQ ID NO: 2; the amino acid sequence of SEQ ID NO: 6; or the amino acid sequence of SEQ ID NO: 7, and wherein the monoclonal antibody has decreased internalization efficiency compared to the internalization efficiency of the monoclonal antibody when prepared under reference culture conditions.

13. The monoclonal antibody of claim 12, wherein the internalization efficiency is 28 ng/ml or lower.

14. The monoclonal antibody of claim 13, wherein the internalization efficiency is 990 ng/ml or lower.

15. The monoclonal antibody of claim 13, wherein the internalization efficiency is 1632 ng/ml or lower.

16. A monoclonal antibody that specifically binds folate receptor alpha (FRA), wherein the monoclonal antibody comprises the heavy chain amino acid sequence in SEQ ID NO: 1 or SEQ ID NO: 5, with or without the c-terminal lysine, and further comprises a light chain amino acid sequence selected from: an amino acid sequence that is 99% identical to the amino acid sequence in SEQ ID NO: 2; the amino acid sequence of SEQ ID NO: 6; or the amino acid sequence of SEQ ID NO: 7, and wherein the monoclonal antibody has altered binding affinity compared to the binding affinity of the monoclonal antibody when prepared under reference culture conditions.

17. The monoclonal antibody of claim 16, wherein the binding affinity is increased.

18. The monoclonal antibody of claim 16, wherein the binding affinity is decreased.

19. The monoclonal antibody of claim 17, wherein the binding affinity is increased 10% or more.

20. The monoclonal antibody of claim 18, wherein the binding affinity is decreased by 10% or more.

21. A composition comprising the antibody according to claim 1.

22. The composition of claim 21, further comprising an additional active agent.

23. A cell culture comprising a eukaryotic host cell, the host cell comprising nucleic acids encoding the amino acid in SEQ ID NO: 1 or SEQ ID NO: 5, and encoding an amino acid sequence selected from: an amino acid sequence that is 99% identical to the amino acid sequence in SEQ ID NO: 2; the amino acid sequence of SEQ ID NO: 6; or the amino acid sequence of SEQ ID NO: 7, wherein the cell culture conditions comprise a parameter selected from the group consisting of galactose, reduced dissolved oxygen tension, reduced temperature, sodium butyrate, copper chloride, high osmolarity and high CO₂.

24. The cell culture of claim 23, further comprising a monoclonal antibody that specifically binds folate receptor alpha (FRA), wherein the monoclonal antibody comprises the heavy chain amino acid sequence in SEQ ID NO: 1 or SEQ ID NO: 5, with or without the c-terminal lysine, and further comprises a light chain amino acid sequence selected from: an amino acid sequence that is 99% identical to the amino acid sequence in SEQ ID NO: 2; the amino acid sequence of SEQ ID NO: 6; or the amino acid sequence of SEQ ID NO: 7, and wherein the monoclonal antibody has altered antibody-dependent cellular cytotoxicity (ADCC) compared to the ADCC of the monoclonal antibody when prepared under reference culture conditions.

25. The cell culture of claim 23, wherein the eukaryotic host cell is a CHO cell.

26. A host cell isolated from the cell culture of claim 24.

27. A monoclonal antibody that specifically binds folate receptor alpha (FRA) isolated from the cell culture of claim 24.

28. A method for producing a monoclonal antibody that specifically binds FRA, wherein the monoclonal antibody comprises the heavy chain amino acid sequence in SEQ ID NO: 1 or SEQ ID NO: 5, with or without the c-terminal lysine, and further comprises a light chain amino acid sequence selected from: an amino acid sequence that is 99% identical to the amino acid sequence in SEQ ID NO: 2; the amino acid sequence of SEQ ID NO: 6; or the amino acid sequence of SEQ ID NO: 7, and where the monoclonal antibody has altered ADCC compared to the ADCC of the monoclonal antibody when prepared under reference culture conditions comprising the step of culturing a CHO cell comprising nucleic acids encoding the heavy chain amino acid sequence and the light chain amino acid sequence in a cell culture conditions comprising a parameter selected from the group consisting of: galactose, reduced dissolved oxygen tension, reduced temperature, sodium butyrate, copper chloride, high osmolarity and high CO₂.

29. The method of claim 28, wherein the ADCC is increased and the culture condition is selected from reduced dissolved oxygen tension and reduced temperature.

30. The method of claim 29, wherein the antibody further has an increased internalization rate.

31. A method for producing a monoclonal antibody that specifically binds FRA, wherein the monoclonal antibody comprises the heavy chain amino acid sequence in SEQ ID NO: 1 or SEQ ID NO: 5, with or without the c-terminal lysine, and further comprises a light chain amino acid sequence selected from: an amino acid sequence that is 99% identical to the amino acid sequence in SEQ ID NO: 2; the amino acid sequence of SEQ ID NO: 6; or the amino acid sequence of SEQ ID NO: 7, and where the monoclonal antibody has an altered internalization rate compared to the internalization rate of the monoclonal antibody when prepared under reference culture conditions comprising the step of culturing a CHO cell comprising nucleic acids encoding the heavy chain amino acid sequence and the light chain amino acid sequence in a cell culture conditions comprising a parameter selected from the group consisting of: galactose, reduced dissolved oxygen tension, reduced temperature, sodium butyrate, copper chloride, high osmolarity, high CO₂, harvest at less than 13 or more than 15 days.

32. The method of claim 31, wherein the internalization rate in increased and the culture condition is selected from reduced dissolved oxygen tension or harvest at less than 13 or more than 15 days.

33. A monoclonal antibody that specifically binds folate receptor alpha (FRA), wherein the monoclonal antibody comprises the heavy chain amino acid sequence in SEQ ID NO: 1 or SEQ ID NO: 5, with or without the c-terminal lysine, and further comprises a light chain amino acid sequence selected from: an amino acid sequence that is 99% identical to the amino acid sequence in SEQ ID NO: 2; the amino acid sequence of SEQ ID NO: 6; or the amino acid sequence of SEQ ID NO: 7, made by a method according to claim 28.

34. A method of reducing the growth of dysplastic cells associated with increased expression of FRA in a subject in need thereof, comprising the step of administering to the subject a monoclonal antibody according to claim 1.

35. The method of claim 34, wherein the dysplastic cells are ovarian, breast, renal, colorectal, lung, endometrial, brain, fallopian tube, uterine cancer cells or leukemia cells.

36. The method of claim 34, wherein the subject is human.

37. A method for treating cancer in a subject in need thereof, comprising the step of administering to the subject a monoclonal antibody according to claim 1.

38. The method of claim 37, wherein the cancer is selected from ovarian, breast, renal, colorectal, lung, endometrial or brain cancer.

39. A method for detecting a cell that expresses FRA, comprising the steps of contacting the cell with an antibody according to claim 1 and detecting binding.

40. A method for detecting FRA in a biological sample, comprising the steps of contacting the biological sample with an antibody according to claim 1 and detecting binding.

41. The method of claim 39, wherein the cell is a dysplastic cell.

42. A composition comprising the antibody according to claim 5.

43. A composition comprising the antibody according to claim 8.

44. A composition comprising the antibody according to claim 12.

45. A composition comprising the antibody according to claim 16.

46. The cell culture of claim 23, further comprising a monoclonal antibody that specifically binds folate receptor alpha (FRA), wherein the monoclonal antibody comprises the heavy chain amino acid sequence in SEQ ID NO: 1 or SEQ ID NO: 5, with or without the c-terminal lysine, and further comprises a light chain amino acid sequence selected from: an amino acid sequence that is 99% identical to the amino acid sequence in SEQ ID NO: 2; the amino acid sequence of SEQ ID NO: 6; or the amino acid sequence of SEQ ID NO: 7, and wherein the monoclonal antibody has an altered internalization rate compared to the internalization rate of the monoclonal antibody when prepared under reference culture conditions.

47. The cell culture of claim 23, further comprising a monoclonal antibody that specifically binds folate receptor alpha (FRA), wherein the monoclonal antibody comprises the heavy chain amino acid sequence in SEQ ID NO: 1 or SEQ ID NO: 5, with or without the c-terminal lysine, and further comprises a light chain amino acid sequence selected from: an amino acid sequence that is 99% identical to the amino acid sequence in SEQ ID NO: 2; the amino acid sequence of SEQ ID NO: 6; or the amino acid sequence of SEQ ID NO: 7, and wherein the monoclonal antibody has an altered N-linked neutral glycan profile compared to the N-linked neutral glycan profile of the monoclonal antibody when prepared under reference culture conditions.

48. The cell culture of claim 23, further comprising a monoclonal antibody that specifically binds folate receptor alpha (FRA), wherein the monoclonal antibody comprises the heavy chain amino acid sequence in SEQ ID NO: 1 or SEQ ID NO: 5, with or without the c-terminal lysine, and further comprises a light chain amino acid sequence selected from: an amino acid sequence that is 99% identical to the amino acid sequence in SEQ ID NO: 2; the amino acid sequence of SEQ ID NO: 6; or the amino acid sequence of SEQ ID NC): 7, and wherein the monoclonal antibody has decreased internalization efficiency compared to the internalization efficiency of the monoclonal antibody when prepared under reference culture conditions.

49. The cell culture of claim 23, further comprising a monoclonal antibody that specifically binds folate receptor alpha (FRA), wherein the monoclonal antibody comprises the heavy chain amino acid sequence in SEQ ID NO: 1 or SEQ ID NO: 5, with or without the c-terminal lysine, and further comprises a light chain amino acid sequence selected from: an amino acid sequence that is 99% identical to the amino acid sequence in SEQ ID NO: 2; the amino acid sequence of SEQ ID NO: 6; or the amino acid sequence of SEQ ID NO: 7, and wherein the monoclonal antibody has altered binding affinity compared to the binding affinity of the monoclonal antibody when prepared under reference culture conditions.

50. A monoclonal antibody that specifically binds folate receptor alpha (FRA), wherein the monoclonal antibody comprises the heavy chain amino acid sequence in SEQ ID NO: 1 or SEQ ID NO: 5, with or without the c-terminal lysine, and further comprises a light chain amino acid sequence selected from: an amino acid sequence that is 99% identical to the amino acid sequence in SEQ ID NO: 2; the amino acid sequence of SEQ ID NO: 6; or the amino acid sequence of SEQ ID NO: 7, made by a method according to claim 31.

51. A method of reducing the growth of dysplastic cells associated with increased expression of FRA in a subject in need thereof, comprising the step of administering to the subject a monoclonal antibody according to claim 5.

52. A method of reducing the growth of dysplastic cells associated with increased expression of FRA in a subject in need thereof, comprising the step of administering to the subject a monoclonal antibody according to claim 8.

53. A method of reducing the growth of dysplastic cells associated with increased expression of FRA in a subject in need thereof, comprising the step of administering to the subject a monoclonal antibody according to claim 12.

54. A method of reducing the growth of dysplastic cells associated with increased expression of FRA in a subject in need thereof, comprising the step of administering to the subject a monoclonal antibody according to claim 16.

55. A method for treating cancer in a subject in need thereof, comprising the step of administering to the subject a monoclonal antibody according to claim 5.

56. A method for treating cancer in a subject in need thereof, comprising the step of administering to the subject a monoclonal antibody according to claim 8.

57. A method for treating cancer in a subject in need thereof, comprising the step of administering to the subject a monoclonal antibody according to claim 12.

58. A method for treating cancer in a subject in need thereof, comprising the step of administering to the subject a monoclonal antibody according to claim 16.

59. A method for detecting a cell that expresses FRA, comprising the steps of contacting the cell with an antibody according to claim 5 and detecting binding.

60. A method for detecting a cell that expresses FRA, comprising the steps of contacting the cell with an antibody according to claim 8 and detecting binding.

61. A method for detecting a cell that expresses FRA, comprising the steps of contacting the cell with an antibody according to claim 12 and detecting binding.

62. A method for detecting a cell that expresses FRA, comprising the steps of contacting the cell with an antibody according to claim 16 and detecting binding.

63. A method for detecting FRA in a biological sample, comprising the steps of contacting the biological sample with an antibody according to claim 5 and detecting binding.

64. A method for detecting FRA in a biological sample, comprising the steps of contacting the biological sample with an antibody according to claim 8 and detecting binding.

65. A method for detecting FRA in a biological sample, comprising the steps of contacting the biological sample with an antibody according to claim 12 and detecting binding.

66. A method for detecting FRA in a biological sample, comprising the steps of contacting the biological sample with an antibody according to claim 16 and detecting binding.


SEQUENCE LISTING (9 sequences)

SEQ ID NO: 1 - protein, 468 aa, Artificial Sequence
  Description of Artificial Sequence Synthetic polypeptide
MGWSCIILFLVATATGVHSEVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 236 aa, Artificial Sequence
  Description of Artificial Sequence Synthetic polypeptide
MGWSCIILFLVATATGVHSDIQLTQSPSSLSASVGDRVTITCSVSSSISSDNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 4 - DNA, 774 bp, Homo sapiens
atggctcagcggatgacaacacagctgctgctccttctagtgtgggtggctgtagtaggggaggctcagacaaggattgcatgggccaggactgagcttctcaatgtctgcatgaacgccaagcaccacaaggaaaagccaggccccgaggacaagttgcatgagcagtgtcgaccctggaggaagaatgcctgctgttctaccaacaccagccaggaagcccataaggatgtttcctacctatatagattcaactggaaccactgtggagagatggcacctgcctgcaaacggcatttcatccaggacacctgcctctacgagtgctcccccaacttggggccctggatccagcaggtggatcagagctggcgcaaagagcgggtactgaacgtgcccctgtgcaaagaggactgtgagcaatggtgggaagattgtcgcacctcctacacctgcaagagcaactggcacaagggctggaactggacttcagggtttaacaagtgcgcagtgggagctgcctgccaacctttccatttctacttccccacacccactgttctgtgcaatgaaatctggactcactcctacaaggtcagcaactacagccgagggagtggccgctgcatccagatgtggttcgacccagcccagggcaaccccaatgaggaggtggcgaggttctatgctgcagccatgagtggggctgggccctgggcagcctggcctttcctgcttagcctggccctaatgctgctgtggctgctcagctag

SEQ ID NO: 5 - protein, 449 aa, Artificial Sequence
  Description of Artificial Sequence Synthetic polypeptide
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 236 aa, Artificial Sequence
  Description of Artificial Sequence Synthetic polypeptide
MGWSCIILFLVATATGVHSDIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 217 aa, Artificial Sequence
  Description of Artificial Sequence Synthetic polypeptide
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - DNA, 1407 bp, Artificial Sequence
  Description of Artificial Sequence Synthetic polynucleotide
atgggatggagctgtatcatcctcttcttggtagcaacagctacaggtgtccactccgaggtccaactggtggagagcggtggaggtgttgtgcaacctggccggtccctgcgcctgtcctgctccgcatctggcttcaccttcagcggctatgggttgtcttgggtgagacaggcacctggaaaaggtcttgagtgggttgcaatgattagtagtggtggtagttatacctactatgcagacagtgtgaagggtagatttgcaatatcgcgagacaacgccaagaacacattgttcctgcaaatggacagcctgagacccgaagacaccggggtctatttttgtgcaagacatggggacgatcccgcctggttcgcttattggggccaagggaccccggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcttatattcaaagctcaccgtggacaagagcaggtggcagcaggggaacgtcttctcatgctccgtgatgcatgaggctctgcacaaccactacacgcagaagagcctctccctgtctcccgggaaatga

SEQ ID NO: 9 - DNA, 711 bp, Artificial Sequence
  Description of Artificial Sequence Synthetic polynucleotide
atgggatggagctgtatcatcctcttcttggtagcaacagctacaggtgtccactccgacatccagctgacccagagcccaagcagcctgagcgccagcgtgggtgacagagtgaccatcacctgtagtgtcagctcaagtataagttccaacaacttgcactggtaccagcagaagccaggtaaggctccaaagccatggatctacggcacatccaacctggcttctggtgtgccaagcagattcagcggtagcggtagcggtaccgactacaccttcaccatcagcagcctccagccagaggacatcgccacctactactgccaacagtggagtagttacccgtacatgtacacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa